(19) United States
(12) Patent Application Publication
(10) Pub. No.: US 2023/0140008 A1
(43) Pub. Date: May 4, 2023

(54) SYSTEMS AND METHODS FOR EVALUATING BIOLOGICAL SAMPLES

(71) Applicant: 10x Genomics, Inc., Pleasanton, CA (US)

(72) Inventors: Eric Siegel, Pleasanton, CA (US); Guy Joseph, Pleasanton, CA (US); Jasper Staab, Pleasanton, CA (US); Jessica Hamel, Pleasanton, CA (US)

(21) Appl. No.: 17/960,037

(22) Filed: Oct. 4, 2022

Related U.S. Application Data
(60) Provisional application No. 63/253,041, filed on Oct. 6, 2021.

(51) Int. Cl. G16B 25/10; G16B 45/00; G16B 30/10

(52) U.S. Cl. CPC G16B 25/10; G16B 30/10; G16B 45/00

(57) ABSTRACT

Systems and methods for evaluating one or more biological samples are provided. A dataset is obtained from nucleic acid sequencing of the biological samples. The dataset comprises a discrete attribute value for each of a plurality of reference sequences for each entity in a plurality of entities in the biological samples. A two-dimensional spatial arrangement of the plurality of entities is indexed, each entity independently assigned a unique two-dimensional position in a k-dimensional binary search tree, and the spatial arrangement is displayed. A user selection of a subset of the displayed arrangement is received. Each entity that is a member of the subset is determined using the k-dimensional binary search tree, thus identifying a subset of entities. Each entity in the subset of entities is assigned to a user-provided category, and the dataset is modified to store an association of each entity in the subset to the category.

### CROSS REFERENCE TO RELATED PATENT APPLICATIONS

This application claims priority to U.S. Provisional Pat. Application No. 63/253,041 entitled “SYSTEMS AND METHODS FOR EVALUATING BIOLOGICAL SAMPLES,” filed Oct. 6, 2021, which is hereby incorporated by reference.

### TECHNICAL FIELD

This specification describes technologies relating to visualizing patterns in large, complex datasets, such as next-generation sequencing data.

### BACKGROUND

The relationship between analytes and cells, including the expression of analytes in populations of cells and/or their relative locations within a tissue sample can be critical to understanding disease pathology. For example, such information can address questions regarding whether lymphocytes are successfully infiltrating a tumor or not, for example by identifying cell surface receptors associated with lymphocytes. In such a situation, lymphocyte infiltration would be associated with a favorable diagnosis whereas the inability of lymphocytes to infiltrate the tumor would be associated with an unfavorable diagnosis. Thus, the relationship of analytes to cell types and/or spatial locations in heterogeneous tissue can be used to analyze biological samples.

Omics technologies including single cell transcriptomics and spatial transcriptomics allow scientists to measure analyte activity (e.g., gene activity) in a biological sample, such as a cell sample or a tissue sample, and map where the analyte activity (e.g., gene activity) is occurring. Already this technology is leading to new discoveries that will prove instrumental in helping scientists gain a better understanding of biological processes and disease.

Single cell transcriptomics and spatial transcriptomics are made possible by advances in nucleic acid sequencing that have given rise to rich datasets for cell populations. Such sequencing techniques provide data for cell populations that can be used to determine genomic heterogeneity, including genomic copy number variation, as well as for mapping clonal evolution (e.g., evaluation of the evolution of tumors).

However, such sequencing datasets are complex and often large, and the techniques used to localize analyte activity (e.g., gene expression) to particular regions or cell populations within a biological sample are labor intensive.

Consequently, there is a need for additional tools to enable a scalable approach to approaching single cell and spatial omics technologies such as single cell transcriptomics, spatial transcriptomics, single cell proteomics, and/or spatial proteomics in a way that allows for improved and less labor intensive analysis, in order to better evaluate heterogeneity in biological samples such as copy number variation, clonal evolution mapping, antigen receptor detection and/or identification of somatic variation in a morphological context.

### SUMMARY

Technical solutions (e.g., computing systems, methods, and non-transitory computer readable storage mediums) for addressing the above-identified problems with discovery patterns in datasets are provided in the present disclosure. In particular, what is needed in the art are visualization systems and corresponding methods, computer systems, and computer-readable storage mediums thereof for the user interaction, analysis, and visualization of large sequencing datasets (e.g., corresponding to at least 1,000, at least 10,000, at least 100,000, or at least 1,000,000 entities in a plurality of entities). Such systems will improve the evaluation of biological samples with respect to such performance measures as speed, responsiveness, and computational robustness (e.g., preventing crashes).

The following presents a summary of the present disclosure in order to provide a basic understanding of some of the aspects of the present disclosure. This summary is not an extensive overview of the present disclosure. It is not intended to identify key/critical elements of the present disclosure or to delineate the scope of the present disclosure. Its sole purpose is to present some of the concepts of the present disclosure in a simplified form as a prelude to the more detailed description that is presented later.

One aspect of the present disclosure provides a visualization system comprising one or more processing cores, a memory, and a display, the memory storing instructions for performing a method for evaluating one or more biological samples. The method comprises obtaining a discrete attribute value dataset derived by nucleic acid sequencing (e.g., single cell or single nuclei sequencing) of the one or more biological samples, where the discrete attribute value dataset comprises a corresponding discrete attribute value for each reference sequence in a plurality of reference sequences for each respective entity in a plurality of entities (e.g., comprising 100,000 entities) in the one or more biological samples. A two-dimensional spatial arrangement of the plurality of entities is indexed, in which each respective entity in the plurality of entities is independently assigned a unique two-dimensional position, in a k-dimensional binary search tree. The two-dimensional spatial arrangement of the plurality of entities is displayed on the display. A user selection of a subset of the two-dimensional spatial arrangement on the display is received. Each entity in the plurality of entities that is a member of the subset is determined using the k-dimensional binary search tree, thereby identifying a subset of entities in the plurality of entities. Each entity in the subset of entities is assigned to a user provided category, and the discrete attribute value dataset is modified to store an association of each respective entity in the subset of entities to the user provided category.

Another aspect of the present disclosure provides a visualization system comprising a main processor, a graphics processing unit, a memory, and a display, the memory storing instructions for using the main processor to perform a method for evaluating one or more biological samples. The method comprises obtaining a discrete attribute value dataset derived by nucleic acid sequencing (e.g., single cell or single nuclei sequencing) of the one or more biological samples, where the discrete attribute value dataset comprises a corresponding discrete attribute value for each reference sequence in a plurality of reference sequences for each respective entity in a plurality of entities (e.g., comprising 100,000 entities) in the one or more biological samples. The plurality of entities is displayed on the display in a two-dimensional spatial arrangement in which each respective entity in the plurality of entities is independently assigned a unique two-dimensional position. A user selection of a subset of the two-dimensional spatial arrangement on the display is received, and, responsive to the user selection, a data structure is created that comprises the unique two-dimensional position of each entity in the subset of entities in the two-dimensional spatial arrangement. The data structure is submitted to the graphics processing unit with a uniform, thereby recoloring the subset of entities on the display in accordance with the uniform.

In some embodiments, the method further comprises clustering the discrete attribute value dataset using the discrete attribute value for each reference sequence in the plurality of reference sequences, or a plurality of dimension reduction components derived therefrom, for each entity in the plurality of entities thereby assigning each respective entity in the plurality of entities to a corresponding cluster in a plurality of clusters, and arranging the plurality of entities into the two-dimensional spatial arrangement based on the clustering.

Another aspect of the present disclosure provides a visualization system comprising one or more processing cores, a memory, and a display, the memory storing instructions for performing a method for evaluating one or more biological samples. The method comprises obtaining a discrete attribute value dataset derived by nucleic acid sequencing (e.g., single cell or single nuclei sequencing) of the one or more biological samples, where the discrete attribute value dataset comprises a corresponding discrete attribute value for each reference sequence in a plurality of reference sequences for each respective entity in a first plurality of entities (e.g., comprising 100,000 entities) in the one or more biological samples. A first spatial projection of the discrete attribute value dataset is displayed in a first window instance, where the first window instance maintains a corresponding state of each respective entity in a second plurality of entities in the first spatial projection, where the second plurality of entities is all or a subset of the first plurality of entities. A second spatial projection of the discrete attribute value dataset is displayed in a second window instance, where the second window instance maintains a corresponding state of each respective entity in a third plurality of entities in the second spatial projection, where the third plurality of entities is all or a subset of the first plurality of entities. A state of each respective entity in a first subset of the second plurality of entities in the first spatial projection is updated in response to a user initiated request for modification of the state of each respective entity in the first subset of the entities in the first spatial projection. A state of each respective entity in the third plurality of entities in the second spatial projection that is in the first subset of entities is selectively updated to match the updated state of the matching entities in the first subset of the second plurality of entities in the first spatial projection.

In some embodiments, each respective entity in the first plurality of entities is assigned a corresponding barcode and the selectively updating a state of each respective entity in the third plurality of entities in the second spatial projection that is in the first subset of entities to match the updated state of the matching entities in the first subset of entities in the first spatial projection comprises matching a respective entity in the third plurality of entities to a corresponding entity in the first subset of entities that has the same barcode as the respective entity. In some embodiments, the corresponding state of each respective entity in the second plurality of entities comprises an identification of which cluster in a plurality of clusters the respective entity is in.

Another aspect of the present disclosure provides a method of evaluating one or more biological samples, using any of the systems disclosed above.

Another aspect of the present disclosure provides a computing system comprising at least one processor and memory storing at least one program to be executed by the at least one processor, the at least one program comprising instructions for evaluating one or more biological samples by any of the methods disclosed above.

Still another aspect of the present disclosure provides a non-transitory computer readable storage medium storing one or more programs for evaluating one or more biological samples. The one or more programs are configured for execution by a computer. The one or more programs collectively encode computer executable instructions for performing any of the methods disclosed above.

Another aspect of the present disclosure provides a visualization system comprising one or more processing cores, a memory, and a display, the memory storing instructions for performing a method for evaluating a first tissue section of a biological sample. The method comprises obtaining a discrete attribute value dataset associated with a plurality of probe spots (e.g., at least 100,000 probe spots), where each probe spot in the plurality of probe spots is assigned a unique barcode in a plurality of barcodes. The discrete attribute value dataset comprises (i) one or more spatial projections of the biological sample, and (ii) a corresponding plurality of discrete attribute values (e.g., at least 500 discrete attribute values) for each respective probe spot in the plurality of probe spots obtained from spatial sequencing of the first tissue section, where each respective discrete attribute value in the corresponding plurality of discrete attribute values is for a different loci in a plurality of loci. A two-dimensional spatial arrangement of the plurality of probe spots is indexed, in which each respective probe spot in the plurality of probe spots is independently assigned a unique two-dimensional position, in a k-dimensional binary search tree. The two-dimensional spatial arrangement of the plurality of probe spots is displayed on the display in accordance with a first spatial projection in the one or more spatial projections. A user selection of a subset of the two-dimensional spatial arrangement on the display is received, and each probe spot in the plurality of probe spots that is a member of the subset is determined using the k-dimensional binary search tree, thereby identifying a subset of probe spots in the plurality of probe spots. Each probe spot in the subset of probe spots is assigned a user provided category; and the discrete attribute value dataset is modified to store an association of each respective probe spot in the subset of probes spots to the user provided category.

Another aspect of the present disclosure provides a visualization system comprising one or more processing cores, a memory, and a display, the memory storing instructions for performing a method for evaluating a first tissue section of a biological sample. The method comprises obtaining a discrete attribute value dataset associated with a plurality of probe spots (e.g., at least 100,000 probe spots), where each probe spot in the plurality of probe spots is assigned a unique barcode in a plurality of barcodes. The discrete attribute value dataset comprises (i) one or more spatial projections of the biological sample, and (ii) a corresponding plurality of discrete attribute values (e.g., at least 500 discrete attribute values) for each respective probe spot in the plurality of probe spots obtained from spatial sequencing of the first tissue section, where each respective discrete attribute value in the corresponding plurality of discrete attribute values is for a different loci in a plurality of loci. The method includes displaying the plurality of probe spots on the display in a two-dimensional spatial arrangement in accordance with a first spatial projection in the one or more spatial projections, with each respective probe spot in the plurality of probe spots independently assigned a unique two-dimensional position in the two-dimensional spatial arrangement. The method further comprises receiving a user selection of a subset of the two-dimensional spatial arrangement on the display, and, responsive to the user selection, creating a data structure that comprises the unique two-dimensional position of each probe spot in the subset of probe spots in the two-dimensional spatial arrangement. The method includes submitting the data structure to the graphics processing unit with a uniform, thereby recoloring the subset of probe spots on the display in accordance with the uniform.

In some embodiments, the one or more spatial projections is a plurality of spatial projections of the biological sample, the plurality of spatial projections comprises the first spatial projection for the first tissue section of the biological sample, and the plurality of spatial projections comprises a second spatial projection for a second tissue section of the biological sample.

In some embodiments, the obtaining comprises clustering all or a subset of the probe spots in the plurality of probe spots across the one or more spatial projections using the discrete attribute values assigned to each respective probe spot in each of the one or more spatial projections as a multi-dimensional vector thereby forming a plurality of clusters.

Another aspect of the present disclosure provides a visualization system comprising one or more processing cores, a memory, and a display, the memory storing instructions for performing a method for evaluating a first tissue section of a biological sample. The method comprises obtaining a discrete attribute value dataset associated with a plurality of probe spots (e.g., at least 100,000 probe spots), where each probe spot in the plurality of probe spots is assigned a unique barcode in a plurality of barcodes. The discrete attribute value dataset comprises (i) a plurality of spatial projections of the biological sample, and (ii) a corresponding plurality of discrete attribute values (e.g., at least 500 discrete attribute values) for each respective probe spot in the plurality of probe spots obtained from spatial sequencing of the first tissue section, where each respective discrete attribute value in the corresponding plurality of discrete attribute values is for a different loci in a plurality of loci. The method includes displaying a first spatial projection of the discrete attribute value dataset in a first window instance, where the first window instance maintains a corresponding state of each respective probe spot in a second plurality of probe spots in the first spatial projection, where the second plurality of probe spots is all or a subset of the first plurality of probe spots. The method further comprises displaying a second spatial projection of the discrete attribute value dataset in a second window instance, where the second window instance maintains a corresponding state of each respective probe spot in a third plurality of probe spots in the second spatial projection, where the third plurality of probe spots is all or a subset of the first plurality of probe spots. The method further comprises updating a state of each respective probe spot in a first subset of the second plurality of probe spots in the first spatial projection in response to a user initiated request for modification of the state of each respective probe spot in the first subset of the probe spots in the first spatial projection, and selectively updating a state of each respective probe spot in the third plurality of probe spots in the second spatial projection that is in the first subset of probe spots to match the updated state of the matching probe spot in the first subset of the second plurality of probe spots in the first spatial projection.

Another aspect of the present disclosure provides a method of evaluating a first tissue section of a biological sample, using any of the systems disclosed above.

Another aspect of the present disclosure provides a computing system comprising at least one processor and memory storing at least one program to be executed by the at least one processor, the at least one program comprising instructions for evaluating a first tissue section of a biological sample by any of the methods disclosed above.

Still another aspect of the present disclosure provides a non-transitory computer readable storage medium storing one or more programs for evaluating a first tissue section of a biological sample. The one or more programs are configured for execution by a computer. The one or more programs collectively encode computer executable instructions for performing any of the methods disclosed above.

As disclosed herein, any embodiment disclosed herein when applicable can be applied to any aspect.

Various embodiments of systems, methods, and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand how the features of various embodiments are used.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIGS. 1A and 1B collectively illustrate an example block diagram illustrating a computing device in accordance with some embodiments of the present disclosure.

FIGS. 2A, 2B, 2C, and 2D collectively illustrate an example method in accordance with an embodiment of the present disclosure, in which optional steps are indicated by dashed lines.

FIG. 3 illustrates a user interface for obtaining a dataset in accordance with some embodiments.

FIG. 4 illustrates an example display in which a heat map that comprises a representation of the differential value for each respective locus in a plurality of loci for each cluster in a plurality of clusters is displayed in a first panel while each respective entity in a plurality of entities is displayed in a second panel in accordance with some embodiments.

FIG. 5 illustrates an example display in which a table that comprises the differential value for each respective locus in a plurality of loci for each cluster in a plurality of clusters is displayed in a first panel while each respective entity in a plurality of entities is displayed in a second panel in accordance with some embodiments.

FIG. 6 illustrates the user selection of classes for a user-defined category and the computation of a heat map of log₂ fold changes in the abundance of mRNA transcripts mapping to individual genes, in accordance with some embodiments of the present disclosure.

FIG. 7 illustrates an example of a user interface where a plurality of entities is displayed in a panel of the user interface, where the spatial location of each entity in the user interface is based upon the physical localization of each entity on a substrate, where each entity is additionally colored in conjunction with one or more clusters identified based on the discrete attribute value dataset, in accordance with some embodiments of the present disclosure.

FIG. 8 illustrates an example of a close-up (e.g., zoomed in) of a region of the entity panel of FIG. 7, in accordance with some embodiments of the present disclosure.

FIGS. 9A and 9B collectively illustrate examples of the image settings available for fine-tuning the visualization of the entity localizations, in accordance with some embodiments of the present disclosure.

FIG. 10 illustrates selection of a single gene for visualization, in accordance with some embodiments of the present disclosure.

FIGS. 11A and 11B illustrate adjusting the opacity of the entities overlaid on an underlying tissue image and creating one or more custom clusters, in accordance with some embodiments of the present disclosure.

FIGS. 12A and 12B collectively illustrate clusters based on t-SNE and UMAP plots in either computational expression space as shown in FIG. 12A or in spatial projection space as shown in FIG. 12B, in accordance with some embodiments of the present disclosure.

FIGS. 13A, 13B, 13C, 13D, 13E, and 13F illustrate spatial projections that make use of linked windows in accordance with an embodiment of the present disclosure.

FIG. 14 illustrates details of a spatial probe spot and capture probe in accordance with an embodiment of the present disclosure.

FIG. 15 illustrates an immunofluorescence image, a representation of all or a portion of each subset of sequence reads at each respective position within one or more images that maps to a respective capture spot corresponding to the respective position, as well as composite representations in accordance with embodiments of the present disclosure.

FIG. 16 illustrates an example visualization system displaying a two-dimensional spatial arrangement of a plurality of entities in a biological sample, in accordance with some embodiments of the present disclosure.

FIG. 17 illustrates an example visualization system displaying a first spatial projection of a discrete attribute value dataset for a plurality of entities in a biological sample in a first window instance and a second spatial projection of the discrete attribute value dataset in a second window instance, in accordance with some embodiments of the present disclosure.

FIGS. 18A and 18B collectively illustrate an example visualization system for user selection of a subset of a two-dimensional spatial arrangement of a plurality of entities on a display and assignment of the user selection of the subset to a user provided category, in accordance with some embodiments of the present disclosure.

FIG. 19 illustrates an example visualization system for selectively updating a state of each respective entity in a subset of entities in a second spatial projection in a second window instance to match an updated state of matching entities in a corresponding subset of entities in a first spatial projection in a first window instance, in accordance with some embodiments of the present disclosure.

FIG. 20 illustrates an example visualization system comprising clustering a discrete attribute value dataset for a plurality of entities and displaying the plurality of entities in a two-dimensional spatial arrangement based on the clustering, in accordance with some embodiments of the present disclosure.

FIG. 21 illustrates an example visualization system for modifying a clustering of a discrete attribute value dataset for a plurality of entities based on barcode selection, in accordance with some embodiments of the present disclosure.

FIGS. 22 and 23 collectively illustrate an example visualization system for modifying a clustering of a discrete attribute value dataset for a plurality of entities based on an adjustment of unique molecular identifier (UMI) thresholds, in accordance with some embodiments of the present disclosure.

FIG. 24 illustrates an example visualization system for modifying a clustering of a discrete attribute value dataset for a plurality of entities based on an adjustment of feature thresholds, in accordance with some embodiments of the present disclosure.

FIGS. 25, 26, 27, and 28 collectively illustrate an example visualization system for modifying a clustering of a discrete attribute value dataset for a plurality of entities using a reclustering workflow, in accordance with some embodiments of the present disclosure.

FIG. 29 illustrates an example visualization system displaying a two-dimensional spatial arrangement of a plurality of entities based on a reclustering procedure, in accordance with some embodiments of the present disclosure.

FIGS. 30A and 30B collectively illustrate an example method in accordance with an embodiment of the present disclosure, in which optional steps are indicated by dashed lines.

FIGS. 31A, 31B, 31C, and 31D collectively illustrate an example method in accordance with an embodiment of the present disclosure, in which optional steps are indicated by dashed lines.

FIGS. 32A, 32B, and 32C collectively illustrate an example method in accordance with an embodiment of the present disclosure, in which optional steps are indicated by dashed lines.

FIG. 33 provides a general schematic workflow illustrating a non-limiting example process for using single cell sequencing technology to generate sequencing data, in accordance with some embodiments of the present disclosure.

FIG. 34 provides a general schematic workflow illustrating a non-limiting example process for using single cell Assay for Transposase Accessible Chromatin (ATAC) sequencing technology to generate sequencing data, in accordance with some embodiments of the present disclosure.

### DETAILED DESCRIPTION

The methods described herein provide for the ability to view, analyze, and/or interact with analyte data in order to evaluate one or more biological samples.

For instance, in some example implementations, the methods described herein provide for the ability to view, analytes, and/or interact with analyte data obtained from single cells (e.g., single nuclei). In particular, in some embodiments, one or more biological samples (e.g., cell suspensions, disaggregated cells, tissues, etc.) are used to generate microfluidic partitions (e.g., droplets), each respective microfluidic partition comprising a respective captured individual cell and a respective capture spot (e.g., a capture bead). In some embodiments, each microfluidic partition is associated with a unique barcode (e.g., where the respective capture spot and/or capture bead for the respective partition is associated with a unique barcode in a plurality of barcodes). In some embodiments, each respective capture spot and/or capture bead comprises one or more capture probes that bind to analytes (e.g., RNA) and/or analyte capture agents that interact with analytes from cells in proximity to (e.g., in contact with and/or partitioned with) the capture spots. In some embodiments, where the analyte is a nucleic acid, sequencing is performed by generating sequencing libraries from the bound nucleic acids (e.g., single cell 3 ’ sequencing, single cell 5 ’ sequencing and/or single cell 5 ’ paired-end sequencing). The sequencing libraries are run on a sequencer and sequencing read data is generated and applied to a sequencing pipeline. Reads from the sequencer are grouped by barcodes and UMIs, and aligned to genes in a transcriptome reference, after which the pipeline generates a number of files, including a feature-barcode matrix. The barcodes correspond to individual capture spots, such as capture spots attached to beads. The value of each entry in the spatial feature-barcode matrix is the number of analytes (e.g., RNA molecules) in proximity to (e.g., in contact with and/or partitioned with) the capture probes and/or beads affixed with that barcode, that align to a particular gene feature. The method then provides for displaying the relative abundance of features (e.g., expression of genes and/or other analytes) for each respective cell (e.g., nucleus) that is partitioned with the respective beads associated with the barcode. This enables users to observe patterns in feature abundance (e.g., gene or protein expression) within a single-cell or cell population context, for the plurality of cells in the one or more biological samples. Such methods provide for, e.g., improved resolution of analyte data.

In other example implementations, the methods described herein provide for the ability to view, analytes, and/or interact with spatial analyte data (e.g., transcriptomics and/or proteomics data) in the original context of the topology of a biological sample. In particular, in some embodiments, one or more biological samples (e.g., fresh-frozen tissue, formalin-fixed paraffin-embedded, etc.) are placed onto a capture area of a substrate (e.g., slide, coverslip, semiconductor wafer, chip, etc.). Each capture area includes preprinted or affixed spots of barcoded capture probes, where each such probe spot has a corresponding unique barcode. The capture area is imaged and then cells within the tissue are permeabilized in place, enabling the capture probes to bind to analytes (e.g., RNA) and/or analyte capture agents that interact with analytes from cells in proximity to (e.g., on top and/or laterally positioned with respect to) the probe spots. In some embodiments, where the analyte is nucleic acids, two-dimensional spatial sequencing is performed by obtaining barcoded cDNA and then sequencing libraries from the bound nucleic acids (e.g., RNA), and the barcoded cDNA is then separated (e.g., washed) from the substrate. The sequencing libraries are run on a sequencer and sequencing read data is generated and applied to a sequencing pipeline. Reads from the sequencer are grouped by barcodes and UMIs, and aligned to genes in a transcriptome reference, after which the pipeline generates a number of files, including a feature-barcode matrix. The barcodes correspond to individual spots within a capture area. The value of each entry in the spatial feature-barcode matrix is the number of analytes (e.g., RNA molecules) in proximity to (e.g., on top and/or laterally positioned with respect to) the probe spot and/or capture probes affixed with that barcode, that align to a particular gene feature. The method then provides for displaying the relative abundance of features (e.g., expression of genes) at each probe spot in the capture area overlaid on the image of the original tissue. This enables users to observe patterns in feature abundance (e.g., gene or protein expression) in the spatial context of the one or more biological samples. Such methods provide for, e.g., improved pathological examination of patient samples.

In some embodiments, the analyte data constitutes a large dataset. For instance, in some embodiments, the analyte data corresponds to at least 1,000, at least 10,000, at least 100,000, or at least 1,000,000 entities in a plurality of entities (e.g., cells). In some such embodiments, analysis of such datasets, including user interaction, modification, spatial analysis, and/or visualization of the analyte data in one or more windows or displays, can result in computational issues such as slow speed, poor responsiveness, and/or system crashes. Accordingly, the present disclosure provides systems and methods for evaluating one or more biological samples that reduces the computational burden on the visualization system, thus improving the performance of the system.

For instance, one aspect of the present disclosure comprises using a k-dimensional binary search tree data structure for selecting regions of a spatial arrangement (e.g., image, visualization, and/or representation) for one or more biological samples. Advantageously, in some embodiments, the k-dimensional binary search tree data structure reduces the complexity of the selection operation on large analyte datasets, reducing the likelihood that a visualization system for analysis of the analyte dataset (e.g., a browser) will freeze or crash and improving the performance of the selection. For instance, the visualization system stores instructions for obtaining a discrete attribute value dataset derived by nucleic acid sequencing (e.g., single cell or single nuclei sequencing) of the one or more biological samples, where the discrete attribute value dataset comprises a corresponding discrete attribute value for each reference sequence in a plurality of reference sequences for each respective entity in a plurality of entities (e.g., at least 100,000 entities) in the one or more biological samples. A two-dimensional spatial arrangement of the plurality of entities is indexed, in which each respective entity in the plurality of entities is independently assigned a unique two-dimensional position, in a k-dimensional binary search tree. The two-dimensional spatial arrangement of the plurality of entities is displayed on the display. A user selection of a subset of the two-dimensional spatial arrangement on the display is received. Each entity in the plurality of entities that is a member of the subset is determined using the k-dimensional binary search tree, thereby identifying a subset of entities in the plurality of entities. Each entity in the subset of entities is assigned to a user provided category, and the discrete attribute value dataset is modified to store an association of each respective entity in the subset of entities to the user provided category.

Another aspect of the present disclosure comprises obtaining a selection data structure separate from the discrete attribute value dataset for the plurality of entities of the one or more biological samples, where the selection data structure stores the two-dimensional positions of each selected data point (e.g., entity) in the two-dimensional spatial arrangement (e.g., image) corresponding to the plurality of entities. Advantageously, in this way, modifications made to selected data points (e.g., entities) such as color changes and/or class or category assignments will result in processing of only the selected data points, rather than of all contiguous data points between selected points, or of all data points in the discrete attribute value dataset. Accordingly, the present disclosure provides a visualization system storing instructions for obtaining a discrete attribute value dataset derived by nucleic acid sequencing (e.g., single cell or single nuclei sequencing) of the one or more biological samples, where the discrete attribute value dataset comprises a corresponding discrete attribute value for each reference sequence in a plurality of reference sequences for each respective entity in a plurality of entities (e.g., comprising 100,000 entities) in the one or more biological samples. The plurality of entities is displayed on the display in a two-dimensional spatial arrangement in which each respective entity in the plurality of entities is independently assigned a unique two-dimensional position. A user selection of a subset of the two-dimensional spatial arrangement on the display is received, and, responsive to the user selection, a data structure is created that comprises the unique two-dimensional position of each entity in the subset of entities in the two-dimensional spatial arrangement. The data structure is submitted to the graphics processing unit with a uniform, thereby recoloring the subset of entities on the display in accordance with the uniform.

Another aspect of the present disclosure comprises performing multi-window comparisons, for a plurality of display windows, using only a selected subset of data points (e.g., entities) in the two-dimensional spatial arrangement (e.g., image) corresponding to the plurality of entities of the one or more biological samples. For instance, in some such embodiments, a minimal action state is compared in each subsequent display window corresponding to only the data, in the discrete attribute value dataset, that matches a selected subset of data points in a first respective display window. Advantageously, this optimization reduces the need to copy all of the data points in the discrete attribute value dataset across each respective window in the plurality of windows each time an action, comparison, and/or modification (e.g., reclustering) is performed on a selected subset of the dataset, thus increasing the speed and efficiency of the visualization system and reducing the likelihood of freezing and crashing. Accordingly, the present disclosure provides a visualization system storing instructions for obtaining a discrete attribute value dataset derived by nucleic acid sequencing (e.g., single cell or single nuclei sequencing) of the one or more biological samples, where the discrete attribute value dataset comprises a corresponding discrete attribute value for each reference sequence in a plurality of reference sequences for each respective entity in a first plurality of entities (e.g., comprising 100,000 entities) in the one or more biological samples. A first spatial projection of the discrete attribute value dataset is displayed in a first window instance, where the first window instance maintains a corresponding state of each respective entity in a second plurality of entities in the first spatial projection, where the second plurality of entities is all or a subset of the first plurality of entities. A second spatial projection of the discrete attribute value dataset is displayed in a second window instance, where the second window instance maintains a corresponding state of each respective entity in a third plurality of entities in the second spatial projection, where the third plurality of entities is all or a subset of the first plurality of entities. A state of each respective entity in a first subset of the second plurality of entities in the first spatial projection is updated in response to a user initiated request for modification of the state of each respective entity in the first subset of the entities in the first spatial projection. A state of each respective entity in the third plurality of entities in the second spatial projection that is in the first subset of entities is selectively updated to match the updated state of the matching entities in the first subset of the second plurality of entities in the first spatial projection.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The implementations described herein provide various technical solutions to evaluate one or more biological samples. An example of such datasets are datasets arising from transcriptome sequencing pipelines that quantify gene expression at particular entities in counts of transcript reads mapped to genes. Details of implementations are now described in conjunction with the Figures.

### Definitions

Specific terminology is used throughout this disclosure to explain various aspects of the apparatus, systems, methods, and compositions that are described. This sub-section includes explanations of certain terms that appear in later sections of the disclosure. To the extent that the descriptions in this section are in apparent conflict with usage in other sections of this disclosure, the definitions in this section will control.

### (A) General Definitions

### Analytes

As used herein, the term “analyte” refers to any biological substance, structure, moiety, or component to be analyzed. The term “target” and/or “feature” is similarly used herein to refer to an analyte of interest or a characteristic thereof. In some embodiments, the apparatus, systems, methods, and compositions described in this disclosure can be used to detect and analyze a wide variety of different analytes.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte is an organelle (e.g., nuclei or mitochondria). In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO2020/176788 and/or U.S. Pat. Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein. In some embodiments, analytes can include one or more intermediate agents, e.g., connected probes or analyte capture agents that bind to nucleic acid, protein, or peptide analytes in a sample.

Cell surface features corresponding to analytes can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis. Examples of nucleic acid analytes include DNA analytes such as genomic DNA, methylated DNA, specific methylated DNA sequences, fragmented DNA, mitochondrial DNA, in situ synthesized PCR products, and RNA/DNA hybrids.

Examples of nucleic acid analytes also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8 S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16 s rRNA or 23 s rRNA).

Additional examples of analytes include mRNA and cell surface features (e.g., using the labelling agents described herein), mRNA and intracellular proteins (e.g., transcription factors), mRNA and cell methylation status, mRNA and accessible chromatin (e.g., ATAC-seq, DNase-seq, and/or MNase-seq), mRNA and metabolites (e.g., using the labelling agents described herein), a barcoded labelling agent (e.g., the oligonucleotide tagged antibodies described herein) and a V(D)J sequence of an immune cell receptor (e.g., T-cell receptor), mRNA and a perturbation agent (e.g., a CRISPR crRNA/sgRNA, TALEN, zinc finger nuclease, and/or antisense oligonucleotide as described herein). In some embodiments, a perturbation agent is a small molecule, an antibody, a drug, an aptamer, a miRNA, a physical environmental (e.g., temperature change), or any other known perturbation agents.

Analytes can include a nucleic acid molecule with a nucleic acid sequence encoding at least a portion of a V(D)J sequence of an immune cell receptor (e.g., a TCR or BCR). In some embodiments, the nucleic acid molecule is cDNA first generated from reverse transcription of the corresponding mRNA, using a poly(T) containing primer. The generated cDNA can then be barcoded using a capture probe, featuring a barcode sequence (and optionally, a UMI sequence) that hybridizes with at least a portion of the generated cDNA. In some embodiments, a template switching oligonucleotide hybridizes to a poly(C) tail added to a 3’ end of the cDNA by a reverse transcriptase enzyme. The original mRNA template and template switching oligonucleotide can then be denatured from the cDNA and the barcoded capture probe can then hybridize with the cDNA and a complement of the cDNA generated. Additional methods and compositions suitable for barcoding cDNA generated from mRNA transcripts including those encoding V(D)J regions of an immune cell receptor and/or barcoding methods and composition including a template switch oligonucleotide are described in PCT Publication No. WO2018/075693 and U.S. Pat. Publication No. US2018-0105808, both of which are incorporated herein by reference in their entireties. V(D)J analysis can also be completed with the use of one or more labelling agents that bind to particular surface features of immune cells and associated with barcode sequences. The one or more labelling agents can include an MHC or MHC multimer.

As described above, the analyte can include a nucleic acid capable of functioning as a component of a gene editing reaction, such as, for example, clustered regularly interspaced short palindromic repeats (CRISPR)-based gene editing. Accordingly, the capture probe can include a nucleic acid sequence that is complementary to the analyte (e.g., a sequence that can hybridize to the CRISPR RNA (crRNA), single guide RNA (sgRNA), or an adapter sequence engineered into a crRNA or sgRNA).

In certain embodiments, an analyte is extracted from a live cell. Processing conditions can be adjusted to ensure that a biological sample remains live during analysis, and analytes are extracted from (or released from) live cells of the sample. Live cell-derived analytes can be obtained only once from the sample or can be obtained at intervals from a sample that continues to remain in viable condition.

In general, the systems, apparatus, methods, and compositions can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual capture spot of the substrate. Methods for performing multiplexed assays to analyze two or more different analytes will be discussed in a subsequent section of this disclosure.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Pat. Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

Additional examples of analytes suitable for use in the present disclosure are described in U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat. Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety.

### Barcodes

As used herein, the term “barcode” refers to a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, a barcode can be or can include a “spatial barcode”. In some embodiments, a barcode includes both a UMI and a spatial barcode. In some embodiments the UMI and barcode are separate entities. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

Barcodes suitable for use in the present disclosure are further described U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat. Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety.

### Bead

The term “bead,” as used herein, generally refers to a particle. In some embodiments, the bead is a solid or semi-solid particle. In some embodiments, the bead is a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. In some embodiments, the bead can be disrupted or dissolved. The bead may be a solid particle (e.g., a metal-based particle including but not limited to iron oxide, gold or silver) covered with a coating comprising one or more polymers. In some embodiments, the coating can be disrupted or dissolved.

As used herein, the term “Gel bead-in-EMulsion” or “GEM” refers to a droplet containing some sample volume and a barcoded gel bead, forming an isolated reaction volume. In some embodiments, when referring to the subset of the sample contained in the droplet, the term “partition” is also used. In various embodiments within the disclosure, the term barcode refers to a GEM containing a gel bead that carries many DNA oligonucleotides with the same barcode, whereas different GEMs have different barcodes. As used herein, the term “GEM well” or “GEM group” refers to a set of partitioned cells (i.e., Gel beads-in-Emulsion or GEMs) from a single 10x Chromium™ Chip channel. One or more sequencing libraries can be derived from a GEM well.

### Biological Samples

As used herein, the term “sample” or “biological sample” refers to any material obtained from a subject for analysis using any of a variety of techniques including, but not limited to, biopsy, surgery, and laser capture microscopy (LCM), and generally includes cells and/or other biological material from the subject. In addition to the subjects described above, a biological sample can also be obtained from non-mammalian organisms (e.g., plants, insects, arachnids, nematodes, fungi, amphibians, and fish. A biological sample can be obtained from a prokaryote such as a bacterium, e.g., Escherichia coli, Staphylococci or Mycoplasma pneumoniae; archaea; a virus such as Hepatitis C virus or human immunodeficiency virus; or a viroid. A biological sample can also be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions and/or disaggregated cells.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

Biological samples can also include fetal cells. For example, a procedure such as amniocentesis can be performed to obtain a fetal cell sample from maternal circulation. Sequencing of fetal cells can be used to identify any of a number of genetic disorders, including, e.g., aneuploidy such as Down’s syndrome, Edwards syndrome, and Patau syndrome. Further, cell surface features of fetal cells can be used to identify any of a number of disorders or diseases.

Biological samples can also include immune cells. Sequence analysis of the immune repertoire of such cells, including genomic, proteomic, and cell surface features, can provide a wealth of information to facilitate an understanding the status and function of the immune system. By way of example, determining the status (e.g., negative or positive) of minimal residue disease (MRD) in a multiple myeloma (MM) patient following autologous stem cell transplantation is considered a predictor of MRD in the MM patient (see, e.g., U.S. Pat. Publication No. 2018/0156784, the entire contents of which are incorporated herein by reference).

Examples of immune cells in a biological sample include, but are not limited to, B cells, T cells (e.g., cytotoxic T cells, natural killer T cells, regulatory T cells, and T helper cells), natural killer cells, cytokine induced killer (CIK) cells, myeloid cells, such as granulocytes (basophil granulocytes, eosinophil granulocytes, neutrophil granulocytes/hyper-segmented neutrophils), monocytes/macrophages, mast cells, thrombocytes/megakaryocytes, and dendritic cells.

As discussed above, a biological sample can include a single analyte of interest, or more than one analyte of interest. Methods for performing multiplexed assays to analyze two or more different analytes in a single biological sample will be discussed in a subsequent section of this disclosure.

A variety of steps can be performed to prepare a biological sample for analysis. Except where indicated otherwise, the preparative steps for biological samples can generally be combined in any manner to appropriately prepare a particular sample for analysis.

For instance, in some embodiments, the biological sample is a tissue section. In some embodiments, the biological sample is prepared using tissue sectioning. A biological sample can be harvested from a subject (e.g., via surgical biopsy, whole subject sectioning, grown in vitro on a growth substrate or culture dish as a population of cells, or prepared for analysis as a tissue slice or tissue section). Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material. The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 micrometers thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 11, 12, 13, 14, 15, 20, 30, 40, or 50 micrometers. Thicker sections can also be used if desired or convenient, e.g., at least 70, 80, 90, or 100 micrometers or more. Typically, the thickness of a tissue section is between 1-100 micrometers, 1-50 micrometers, 1-30 micrometers, 1-25 micrometers, 1-20 micrometers, 1-15 micrometers, 1-10 micrometers, 2-8 micrometers, 3-7 micrometers, or 4-6 micrometers, but as mentioned above, sections with thicknesses larger or smaller than these ranges can also be analyzed.

In some embodiments, a tissue section is a similar size and shape to a substrate (e.g., the first substrate and/or the second substrate). In some embodiments, a tissue section is a different size and shape from a substrate. In some embodiments, a tissue section is on all or a portion of the substrate. In some embodiments, several biological samples from a subject are concurrently analyzed. For instance, in some embodiments several different sections of a tissue are concurrently analyzed. In some embodiments, 1, 2, 3, 4, 5, 6, 7, 8, 9, 10, 11, 12, 13, 14, 15, 16, 17, 18, 19, or 20 different biological samples from a subject are concurrently analyzed. For example, in some embodiments, 1, 2, 3, 4, 5, 6, 7, 8, 9, 10, 11, 12, 13, 14, 15, 16, 17, 18, 19, or 20 different tissue sections from a single biological sample from a single subject are concurrently analyzed. In some embodiments, one or more images are acquired of each such tissue section.

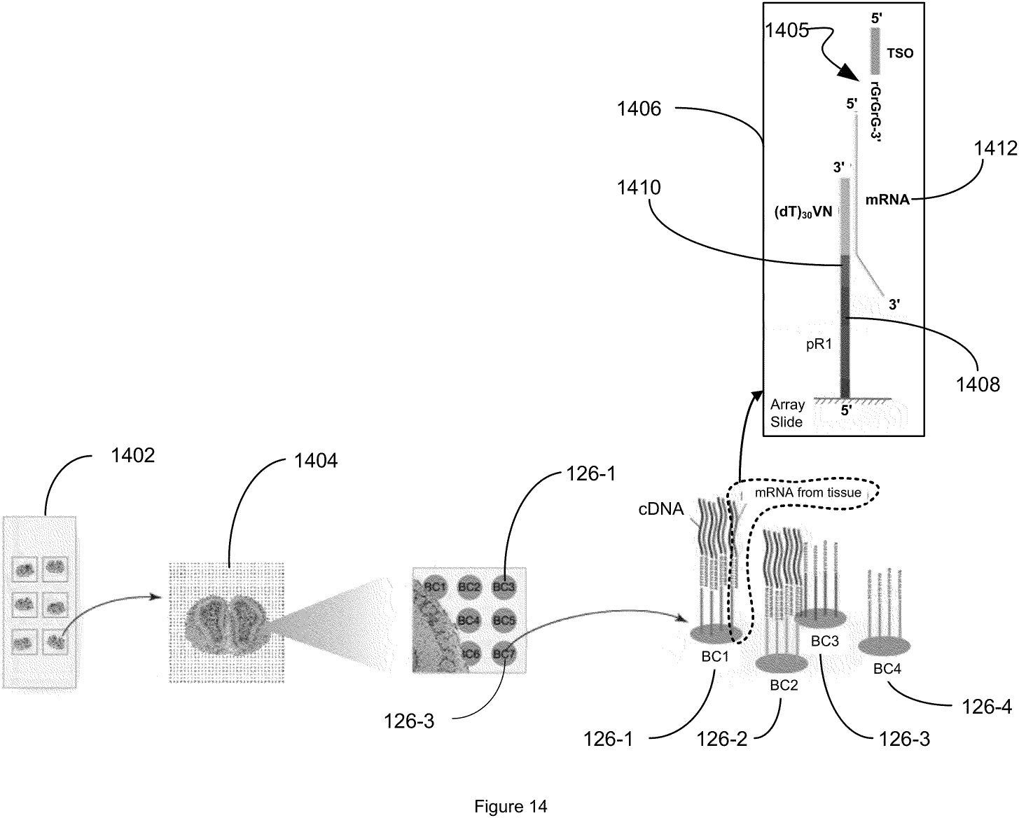

In some embodiments, a tissue section on a substrate is a single uniform section. In some embodiments, multiple tissue sections are on a substrate. In some such embodiments, a single capture area such as capture area 1402 on a substrate, as illustrated in FIG. 14, can contain multiple tissue sections 1404, where each tissue section is obtained from either the same biological sample and/or subject or from different biological samples and/or subjects. In some embodiments, a tissue section is a single tissue section that comprises one or more regions where no cells are present (e.g., holes, tears, or gaps in the tissue). Thus, in some embodiments, such as the above, an image of a tissue section on a substrate can contain regions where tissue is present and regions where tissue is not present.

Additional examples of tissue samples are shown in Table 1 and catalogued, for example, in 10X, 2019, “Visium Spatial Gene Expression Solution,” and in U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat. Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety.

TABLE 1

Examples of tissue samples

| | | |
|---|---|---|
| Organism | Tissue | Healthy/Diseased |
| Human | Brain | Cerebrum Glioblastoma Multiforme |
| Human | Breast | Healthy |
| Human | Breast | Invasive Ductal Carcinoma |
| Human | Breast | Invasive Lobular Carcinoma |
| Human | Heart | Healthy |
| Human | Kidney | Healthy |
| Human | Kidney | Nephritis |
| Human | Large Intestine | Colorectal Cancer |
| Human | Lung | Papillary Carcinoma |
| Human | Lymph Node | Healthy |
| Human | Lymph Node | Inflamed |
| Human | Ovaries | Tumor |
| Human | Spleen | Inflamed |
| Mouse | Brain | Healthy |
| Mouse | Eyes | Healthy |
| Mouse | Heart | Healthy |
| Mouse | Kidney | Healthy |
| Mouse | Large Intestine | Healthy |
| Mouse | Liver | Healthy |
| Mouse | Lungs | Healthy |
| Mouse | Ovary | Healthy |
| Mouse | Quadriceps | Healthy |
| Mouse | Small Intestine | Healthy |
| Mouse | Spleen | Healthy |
| Mouse | Stomach | Healthy |
| Mouse | Testes | Healthy |
| Mouse | Thyroid | Healthy |
| Mouse | Tongue | Healthy |
| Rat | Brain | Healthy |
| Rat | Heart | Healthy |
| Rat | Kidney | Healthy |
| Mouse | Tongue | Healthy |
| Rat | Brain | Healthy |
| Rat | Heart | Healthy |
| Rat | Kidney | Healthy |

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analyzed successively to obtain three-dimensional information about the biological sample.

In some embodiments, a biological sample is prepared using one or more steps including, but not limited to, freezing, fixation, embedding, formalin fixation and paraffin embedding, hydrogel embedding, biological sample transfer, isometric expansion, cell disaggregation, cell suspension, cell adhesion, permeabilization, lysis, protease digestion, selective permeabilization, selective lysis, selective enrichment, enzyme treatment, library preparation, and/or sequencing pre-processing. Methods for biological sample preparation that are contemplated in the present disclosure are described in further detail in U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat. Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety.

In some embodiments, a biological sample is prepared by staining. To facilitate visualization, biological samples can be stained using a wide variety of stains and staining techniques. In some embodiments, for example, a sample can be stained using any number of biological stains, including but not limited to, acridine orange, Bismarck brown, carmine, Coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or a combination thereof.

The sample can be stained using known staining techniques, including Can-Grunwald, Giemsa, hematoxylin and eosin (H&E), Jenner’s, Leishman, Masson’s trichrome, Papanicolaou, Romanowsky, silver, Sudan, Wright’s, and/or Periodic Acid Schiff (PAS) staining techniques. PAS staining is typically performed after formalin or acetone fixation.

In some embodiments, the sample is stained using a detectable label (e.g., radioisotopes, fluorophores, chemiluminescent compounds, bioluminescent compounds, and dyes). In some embodiments, a biological sample is stained using only one type of stain or one technique. In some embodiments, staining includes biological staining techniques such as H&E staining. In some embodiments, staining includes identifying analytes using fluorescently-labeled antibodies. In some embodiments, a biological sample is stained using two or more different types of stains, or two or more different staining techniques. For example, a biological sample can be prepared by staining and imaging using one technique (e.g., H&E staining and bright-field imaging), followed by staining and imaging using another technique (e.g., IHC/IF staining and fluorescence microscopy) for the same biological sample.

In some embodiments, biological samples can be destained. Methods of destaining or discoloring a biological sample are known in the art, and generally depend on the nature of the stain(s) applied to the sample. For example, H&E staining can be destained by washing the sample in HC1, or any other low pH acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in a low pH acid (e.g., HCl). In some embodiments, destaining can include adding HCl to a downstream solution (e.g., permeabilization solution). In some embodiments, destaining can include dissolving an enzyme used in the disclosed methods (e.g., pepsin) in a low pH acid (e.g., HCl) solution. In some embodiments, after destaining hematoxylin with a low pH acid, other reagents can be added to the destaining solution to raise the pH for use in other applications. For example, SDS can be added to a low pH acid destaining solution in order to raise the pH as compared to the low pH acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., 2017, J. Histochem. Cytochem. 65(8): 431-444, Lin et al., 2015, Nat Commun. 6:8390, Pirici et al., 2009, J. Histochem. Cytochem. 57:567-75, and Glass et al., 2009, J. Histochem. Cytochem. 57:899-905, the entire contents of each of which are incorporated herein by reference.

In some embodiments, the biological sample can be attached to a substrate (e.g., a slide and/or a chip). Examples of substrates suitable for this purpose are described in detail elsewhere herein (see, for example, Definitions: “Substrates,” below). Attachment of the biological sample can be irreversible or reversible, depending upon the nature of the sample and subsequent steps in the analytical method.

In certain embodiments, the sample can be attached to the substrate reversibly by applying a suitable polymer coating to the substrate and contacting the sample to the polymer coating. The sample can then be detached from the substrate using an organic solvent that at least partially dissolves the polymer coating. Hydrogels are examples of polymers that are suitable for this purpose. More generally, in some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

Biological samples contemplated for use in the present disclosure are further described in U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat. Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety.

### Capture Probes

A “capture probe,” also interchangeably referred to herein as a “probe,” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe is a conjugate (e.g., an oligonucleotide-antibody conjugate). In some embodiments, the capture probe includes a barcode (e.g., a spatial barcode and/or a unique molecular identifier (UMI)) and a capture domain.

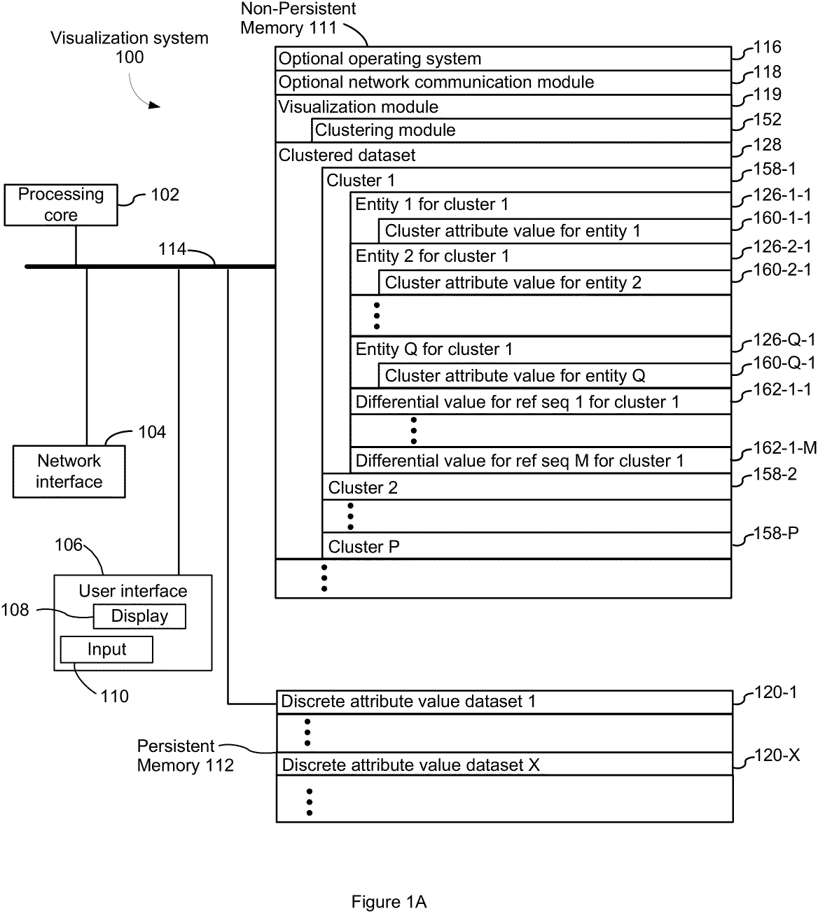

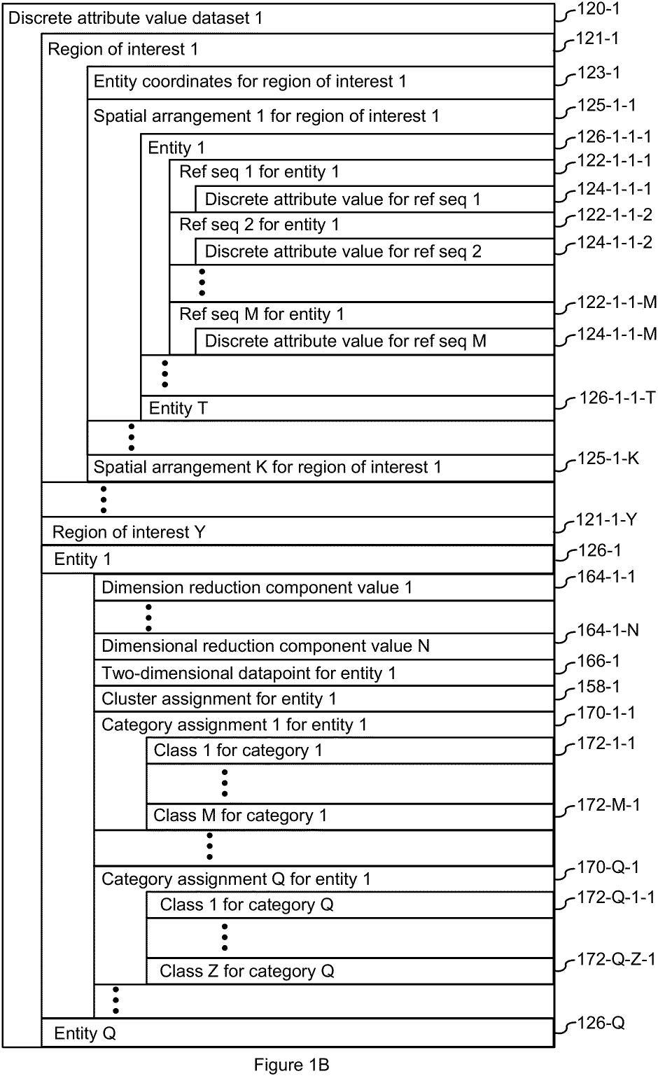

In some embodiments, the capture probe is optionally coupled to a capture spot (e.g., a probe spot 126, as illustrated in FIGS. 1A-C and 14), for instance, by a cleavage domain, such as a disulfide linker.

The capture probe can include functional sequences that are useful for subsequent processing, which can include a sequencer specific flow cell attachment sequence, e.g., a P5 sequence, and/or sequencing primer sequences, e.g., an R1 primer binding site, an R2 primer binding site. In some embodiments, a sequencer specific flow cell attachment sequence is a P7 sequence and sequencing primer sequence is a R2 primer binding site.

A barcode 1408 can be included within the capture probe for use in barcoding the target analyte. The functional sequences can be selected for compatibility with a variety of different sequencing systems, e.g., 454 Sequencing, Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the barcode 1408 and/or functional sequences (e.g., flow cell attachment sequence and/or sequencing primer sequences) can be common to all of the probes attached to a given capture spot. The barcode can also include a capture domain to facilitate capture of a target analyte.

Other aspects of capture probes contemplated for use in the present disclosure are known in the art. For instance, example suitable cleavage domains are described in further detail in PCT Publication No. 202020176788A1, entitled “Profiling of biological analytes with spatially barcoded oligonucleotide arrays,” the entire contents of which is incorporated herein by reference. Example suitable functional domains are described in further detail in U.S. Pat. Application No. 16/992,569, entitled “Systems and Methods for Using the Spatial Distribution of Haplotypes to Determine a Biological Condition,” filed Aug. 13, 2020, as well as PCT Publication No. 202020176788A1, entitled “Profiling of biological analytes with spatially barcoded oligonucleotide arrays,” each of which is hereby incorporated herein by reference. Example suitable spatial barcodes and unique molecular identifiers are described in further detail in U.S. Pat. Application No. 16/992,569, entitled “Systems and Methods for Using the Spatial Distribution of Haplotypes to Determine a Biological Condition,” filed Aug. 13, 2020, and PCT Publication No. 202020176788A1, entitled “Profiling of biological analytes with spatially barcoded oligonucleotide arrays,” each of which is hereby incorporated herein by reference.

Capture probes contemplated for use in the present disclosure are further described U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat. Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety.

### Capture Spots

As used interchangeably herein, the terms “capture spot,” “probe spot,” “capture feature,” “capture area,” or “capture probe plurality” refer to an entity that acts as a support or repository for various molecular entities used in sample analysis. Examples of capture spots include, but are not limited to, a bead, a spot of any two- or three-dimensional geometry (e.g., an inkjet spot, a masked spot, a square on a grid), a well, and a hydrogel pad. In some embodiments, a capture spot is an area on a substrate at which capture probes labelled with spatial barcodes are clustered. Specific non-limiting embodiments of capture spots and substrates are further described below in the present disclosure.

In some embodiments, capture spots are directly or indirectly attached or fixed to a substrate (e.g., of a chip or a slide). In some embodiments, the capture spots are not directly or indirectly attached or fixed to a substrate, but instead, for example, are disposed within an enclosed or partially enclosed three dimensional space (e.g., wells or divots). In some embodiments, some or all capture spots in an array include a capture probe.

In some embodiments, a capture spot includes different types of capture probes attached to the capture spot. For example, the capture spot can include a first type of capture probe with a capture domain designed to bind to one type of analyte, and a second type of capture probe with a capture domain designed to bind to a second type of analyte. In general, capture spots can include one or more (e.g., two or more, three or more, four or more, five or more, six or more, eight or more, ten or more, 12 or more, 15 or more, 20 or more, 30 or more, 50 or more) different types of capture probes attached to a single capture spot.

In some embodiments, each respective probe spot in a plurality of probe spots is a physical probe spot (e.g., on a substrate). In some embodiments, a respective probe spot in a plurality of probe spots is a visual representation of a physical probe spot, such as an image of the probe spot and/or a two-dimensional position of the respective probe spot in a two-dimensional spatial arrangement of the plurality of probe spots.

In some embodiments, each respective probe at each respective probe spot is associated with a unique corresponding barcode. In some embodiments, each probe spot in the plurality of probe spots has a corresponding respective barcode, where each barcode is uniquely identifiable. The location of each barcode is known with regard to each other barcode (e.g., barcodes are spatially coded). An example of such measurement techniques for spatial probe spot based sequencing is disclosed in U.S. Pat. Publication Nos. US 2021-0062272, entitled “Systems and Methods for Using the Spatial Distribution of Haplotypes to Determine a Biological Condition,” and US 2021-0155982, entitled “Pipeline for Analysis of Analytes,” each of which is hereby incorporated by reference. In some embodiments, each respective probe spot comprises a plurality of corresponding probes with different corresponding barcodes.

In some embodiments, a capture spot on the array includes a bead. In some embodiments, two or more beads are dispersed onto a substrate to create an array, where each bead is a capture spot on the array. Beads can optionally be dispersed into wells on a substrate, e.g., such that only a single bead is accommodated per well.

Further details and non-limiting embodiments relating to capture spots, including but not limited to beads, bead arrays, bead properties (e.g., structure, materials, construction, cross-linking, degradation, reagents, and/or optical properties), and for covalently and non-covalently bonding beads to substrates are described in U.S. Pat. Publication No. US 2021-0062272, entitled “SYSTEMS AND METHODS FOR USING THE SPATIAL DISTRIBUTION OF HAPLOTYPES TO DETERMINE A BIOLOGICAL CONDITION, U.S. Pat. Publication No. 20110059865A1 entitled “Modified Molecular Arrays”, U.S. Provisional Application No. 62/839,346, U.S. Pat. No. 9,012,022, and PCT publication 202020176788A1, entitled “Profiling of biological analytes with spatially barcoded oligonucleotide arrays”; U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat. Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety.

### Capture Spot Arrays

In some embodiments, capture spots are collectively positioned on a substrate. As used herein, the term “capture spot array” or “array” refers to a specific arrangement of a plurality of capture spots (also termed “features”) that is either irregular or forms a regular pattern. Individual capture spots in the array differ from one another based on their relative spatial locations. In general, at least two of the plurality of capture spots in the array include a distinct capture probe (e.g., any of the examples of capture probes described herein).

Arrays can be used to measure large numbers of analytes simultaneously. In some embodiments, oligonucleotides are used, at least in part, to create an array. For example, one or more copies of a single species of oligonucleotide (e.g., capture probe) can correspond to or be directly or indirectly attached to a given capture spot in the array. In some embodiments, a given capture spot in the array includes two or more species of oligonucleotides (e.g., capture probes). In some embodiments, the two or more species of oligonucleotides (e.g., capture probes) attached directly or indirectly to a given capture spot on the array include a common (e.g., identical) spatial barcode.

In some embodiments, a substrate and/or an array (e.g., two-dimensional array) comprises a plurality of capture spots. In some embodiments, a substrate and/or an array includes between 4000 and 10,000 capture spots, or any range within 4000 to 6000 capture spots. For example, a substrate and/or an array includes between 4,000 to 4,400 capture spots, 4,000 to 4,800 capture spots, 4,000 to 5,200 capture spots, 4,000 to 5,600 capture spots, 5,600 to 6,000 capture spots, 5,200 to 6,000 capture spots, 4,800 to 6,000 capture spots, or 4,400 to 6,000 capture spots. In some embodiments, the substrate and/or array includes between 4,100 and 5,900 capture spots, between 4,200 and 5,800 capture spots, between 4,300 and 5,700 capture spots, between 4,400 and 5,600 capture spots, between 4,500 and 5,500 capture spots, between 4,600 and 5,400 capture spots, between 4,700 and 5,300 capture spots, between 4,800 and 5,200 capture spots, between 4.900 and 5,100 capture spots, or any range within the disclosed sub-ranges. For example, the substrate and/or array can include about 4,000 capture spots, about 4,200 capture spots, about 4,400 capture spots, about 4,800 capture spots, about 5,000 capture spots, about 5.200 capture spots, about 5,400 capture spots, about 5,600 capture spots, or about 6,000 capture spots. In some embodiments, the substrate and/or array comprises at least 4,000 capture spots. In some embodiments, the substrate and/or array includes approximately 5,000 capture spots.

Arrays suitable for use in the present disclosure are further described in PCT Publication No. 202020176788A1, entitled “Profiling of biological analytes with spatially barcoded oligonucleotide arrays”; in U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat. Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety.

### Contact

As used herein, the terms “contact,” “contacted,” and/ or “contacting” of a biological sample with a substrate comprising capture spots refers to any contact (e.g., direct or indirect) such that capture probes can interact (e.g., capture) with analytes from the biological sample. For example, the substrate may be near or adjacent to the biological sample without direct physical contact, yet capable of capturing analytes from the biological sample. In some embodiments the biological sample is in direct physical contact with the substrate. In some embodiments, the biological sample is in indirect physical contact with the substrate. For example, a liquid layer may be between the biological sample and the substrate. In some embodiments, the analytes diffuse through the liquid layer. In some embodiments the capture probes diffuse through the liquid layer. In some embodiments, reagents may be delivered via the liquid layer between the biological sample and the substrate. In some embodiments, indirect physical contact may be the presence of a second substrate (e.g., a hydrogel, a film, a porous membrane) between the biological sample and the first substrate comprising capture spots with capture probes. In some embodiments, reagents are delivered by the second substrate to the biological sample.

In some embodiments, a cell immobilization agent can be used to contact a biological sample with a substrate (e.g., by immobilizing non-aggregated or disaggregated sample on a spatially-barcoded array prior to analyte capture). A “cell immobilization agent” as used herein can refer to an agent (e.g., an antibody), attached to a substrate, which can bind to a cell surface marker. Non-limiting examples of a cell surface marker include CD45, CD3, CD4, CD8, CD56, CD19, CD20, CD11c, CD14, CD33, CD66b, CD34, CD41, CD61, CD235a, CD146, and epithelial cellular adhesion molecule (EpCAM). A cell immobilization agent can include any probe or component that can bind to (e.g., immobilize) a cell or tissue when on a substrate. A cell immobilization agent attached to the surface of a substrate can be used to bind a cell that has a cell surface maker. The cell surface marker can be a ubiquitous cell surface marker, wherein the purpose of the cell immobilization agent is to capture a high percentage of cells within the sample. The cell surface marker can be a specific, or more rarely expressed, cell surface marker, wherein the purpose of the cell immobilization agent is to capture a specific cell population expressing the target cell surface marker. Accordingly, a cell immobilization agent can be used to selectively capture a cell expressing the target cell surface marker from a population of cells that do not have the same cell surface marker.

Generally, analytes can be captured when contacting a biological sample with, e.g., a substrate comprising capture probes (e.g., substrate with capture probes embedded, spotted, printed on the substrate or a substrate with capture spots (e.g., beads, wells) comprising capture probes). Capture can be performed using passive capture methods and/or active capture methods.

In some embodiments, capture of analytes is facilitated by treating the biological sample with permeabilization reagents. If a biological sample is not permeabilized sufficiently, the amount of analyte captured on the substrate can be too low to enable adequate analysis. Conversely, if the biological sample is too permeable, the analyte can diffuse away from its origin in the biological sample, such that the relative spatial relationship of the analytes within the biological sample is lost. Hence, a balance between permeabilizing the biological sample enough to obtain good signal intensity while still maintaining the spatial resolution of the analyte distribution in the biological sample is desired. Methods of preparing biological samples to facilitate capture are known in the art and can be modified depending on the biological sample and how the biological sample is prepared (e.g., fresh frozen, FFPE, etc.). Examples of analyte capture suitable for use in the present disclosure are further described in U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat. Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety

### Entity

As used herein, the term “entity” refers to a unit of analysis, such as a group of analytes. In some embodiments, an entity is a unit of a biological sample, such as a cell or a nucleus. In some embodiments, an entity describes a single cell comprising a cell nucleus. In some embodiments, each respective entity in a plurality of entities is a single cell in a plurality of single cells (e.g., a cell suspension and/or a plurality of disaggregated cells from a biological sample). In some such embodiments, each respective cell in the plurality of cells comprises a respective nucleus that characterizes the respective cell as a distinct unit of the biological sample (e.g., a cell in a tissue section). An entity can refer to a unit in a physical form (e.g., a physical cell in or obtained from a biological sample) or a representation thereof, such as a set of data originating from the unit and/or a visual representation of the unit (e.g., an image of a single cell, a two-dimensional spatial arrangement of data associated with the single cell, etc.).

In some embodiments, the term “entity” is used to refer to a sub-cellular region of a cell (e.g., an individual cell comprising a respective cell nucleus). Sub-cellular regions include, but are not limited to, cell nuclei, mitochondria, cytosol, microsomes, and more generally, any other compartment, organelle, or portion of a cell. Accordingly, in some embodiments, each respective entity in a plurality of entities is a respective cell nucleus of a single cell in a plurality of single cells.

In some embodiments, the term “entity” is used to describe a discrete unit of analytes obtained from a biological sample, such as a set of analytes originating from a single cell. In some such embodiments, the term “entity” refers to the discrete unit of analytes in physical form or a representation thereof, such as a set of data originating from a measurement or analysis of the set of analytes and/or a visual representation of the set of analytes (e.g., a two-dimensional spatial arrangement of data that represents the set of analytes). The discrete unit of analytes can comprise a single type of analyte or a combination of different types of analytes (e.g., DNA, RNA, proteins, or a combination thereof). In some embodiments, the discrete unit of analytes (and/or the representation thereof) is obtained using one or more capture probes specific to each respective analyte in the discrete unit of analytes. In some such embodiments, the discrete unit of analytes (and/or the representation thereof) is obtained from a nucleic acid sequencing. In some embodiments, the discrete unit of analytes (and/or the representation thereof) is obtained from a single nucleus-based nucleic acid sequencing, such as single nuclei RNA sequencing (snRNA-seq). For instance, snRNA-seq can be used to measure RNA expression from isolated nuclei as opposed to RNA of an entire cell (e.g., cytoplasmic RNA plus nuclear RNA). See, for example, Grindberg et al., (2013), “RNA-sequencing from single nuclei,” Proc. Natl Acad. Sci. USA, 110, 19802-19807; Lacar et al., (2016), “Nuclear RNA-seq of single neurons reveals molecular signatures of activation,” Nature Comm., 7:11022; Basile et al., (2021) “Using single-nucleus RNA-sequencing to interrogate transcriptomic profiles of archived human pancreatic islets,” Genome Medicine 13:128, each of which is hereby incorporated herein by reference in its entirety.

In other such embodiments, the discrete unit of analytes (and/or the representation thereof) is obtained from single cell nucleic acid sequencing. Single cell nucleic acid sequencing can include, for instance, single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, and any combination thereof. The sequencing technique can be selected based on the desired analyte to be measured. For instance, scRNA-seq, scTag-seq, and miRNA-seq can be used to measure RNA expression. Specifically, scRNA-seq measures expression of RNA transcripts, scTag-seq allows detection of rare mRNA species, and miRNA-seq measures expression of micro-RNAs. CyTOF/SCoP and E-MS/Abseq can be used to measure protein expression in the cell. CITE-seq simultaneously measures both gene expression and protein expression in the cell, and scATAC-seq measures chromatin conformation in the cell. See, for example, Olsen et al., (2018), “Introduction to Single-Cell RNA Sequencing,” Current protocols in molecular biology 122(1), pg. 57; Rozenberg et al., (2016), “Digital gene expression analysis with sample multiplexing and PCR duplicate detection: A straightforward protocol,” BioTechniques, 61(1), pg. 26; Buenrostro et al., (2015), “ATAC-seq: a method for assaying chromatic accessibility genome-wide,” Current protocols in molecular biology, 109(1), pg. 21; Faridani et al., (2016), “Single-cell sequencing of the small-RNA transcriptome,” Nature biotechnology, 34(12), pg. 1264; Bandura et al., (2009), “Mass cytometry: technique for real time single cell multitarget immunoassay based on inductively coupled plasma time-of-flight mass spectrometry,” Analytic chemistry, 81(16), pg. 6813; Budnik et al., (2018), “SCoPE-ME: mass spectrometry of single mammalian cells quantifies proteome heterogeneity during cell differentiation,” Genome biology, 19(1), pg. 161; Shahi et al., (2017), “Abseq: Ultra high-throughput single cell protein profiling with droplet microfluidic barcoding,” Scientific reports, 7, pg. 44447; and Stoeckius et al., (2017), “Simultaneous epitope and transcriptome measurement in single cells,” Nature Methods, 14(9), pg. 856, each of which is hereby incorporated herein by reference in its entirety.

In some embodiments, an entity is characterized by a barcode. For instance, in some embodiments, each respective entity in a plurality of entities is associated with a unique respective barcode in a plurality of barcodes. In some embodiments, each respective entity in a plurality of entities is associated with a unique respective subset of barcodes in a plurality of subsets of barcodes (e.g., each respective entity is associated with a plurality of barcodes). In some embodiments, two or more entities are associated with the same barcode.

In some embodiments, a respective entity corresponds to one or more respective probe spots in a plurality of probe spots. In some embodiments, each respective probe spot in a plurality of probe spots corresponds to one or more respective entities in the plurality of entities (see, e.g., Definitions: Capture Spots, above), for instance, where an entity is another unit of analysis, such as a cell. A respective probe spot can be larger than an entity (e.g., a probe spot can encompass one or more entities) or smaller than an entity (e.g., an entity can encompass one or more probe spots). Thus, an entity can refer to a respective one or more probe spots, a respective unit of capture probes that are in contact with a respective single cell, the respective unit of analytes captured thereby, and/or the respective unit of data obtained therefrom. Similarly, an entity can refer to a representation thereof, such as a set of data originating from an analysis of analyte data captured by the unit of capture probes and/or a visual representation thereof (e.g., a two-dimensional spatial arrangement of analyte data).

Any methods and/or embodiments comprising the capture, analysis, arrangement, and/or visualization of a plurality of a first type of entity (e.g., nuclei) for one or more biological samples disclosed herein can be similarly applied to a plurality of a second type of entity (e.g., probe spots) for the one or more biological samples. Similarly, in some embodiments, any methods and/or embodiments comprising the capture, analysis, arrangement, and/or visualization of the plurality of a second type of entity (e.g., probe spots) for the one or more biological samples disclosed herein can be similarly applied to a plurality of a first type of entity (e.g., nuclei) for the one or more biological samples.

### Fiducials

As used interchangeably herein, the terms “fiducial,” “spatial fiducial,” “fiducial marker,” and “fiducial spot” generally refers to a point of reference or measurement scale. In some embodiments, imaging is performed using one or more fiducial markers, i.e., objects placed in the field of view of an imaging system that appear in the image produced. Fiducial markers can include, but are not limited to, detectable labels such as fluorescent, radioactive, chemiluminescent, calorimetric, and colorimetric labels. The use of fiducial markers to stabilize and orient biological samples is described, for example, in Carter et al., Applied Optics 46:421-427, 2007), the entire contents of which are incorporated herein by reference.

In some embodiments, a fiducial marker can be present on a substrate to provide orientation of the biological sample. In some embodiments, a microsphere can be coupled to a substrate to aid in orientation of the biological sample. In some examples, a microsphere coupled to a substrate can produce an optical signal (e.g., fluorescence). In another example, a microsphere can be attached to a portion (e.g., corner) of an array in a specific pattern or design (e.g., hexagonal design) to aid in orientation of a biological sample on an array of capture spots on the substrate. In some embodiments, a fiducial marker can be an immobilized molecule with which a detectable signal molecule can interact to generate a signal. For example, a marker nucleic acid can be linked or coupled to a chemical moiety capable of fluorescing when subjected to light of a specific wavelength (or range of wavelengths). Such a marker nucleic acid molecule can be contacted with an array before, contemporaneously with, or after the tissue sample is stained to visualize or image the tissue section. In some embodiments, it can be advantageous to use a marker that can be detected using the same conditions (e.g., imaging conditions) used to detect an analyte of interest.

In some embodiments, fiducial markers are included to facilitate the orientation of a tissue sample or an image thereof in relation to an immobilized capture probes on a substrate. Any number of methods for marking an array can be used such that a marker is detectable only when a tissue section is imaged. For instance, a molecule, e.g., a fluorescent molecule that generates a signal, can be immobilized directly or indirectly on the surface of a substrate. Markers can be provided on a substrate in a pattern (e.g., an edge, one or more rows, one or more lines, etc.).

In some embodiments, a fiducial marker can be stamped, attached, or synthesized on the substrate and contacted with a biological sample. Typically, an image of the sample and the fiducial marker is taken, and the position of the fiducial marker on the substrate can be confirmed by viewing the image.

In some examples, fiducial markers can surround the array. In some embodiments the fiducial markers allow for detection of, e.g., mirroring. In some embodiments, the fiducial markers may completely surround the array. In some embodiments, the fiducial markers may not completely surround the array. In some embodiments, the fiducial markers identify the corners of the array. In some embodiments, one or more fiducial markers identify the center of the array.

Example spatial fiducials suitable for use in the present disclosure are further described in in U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat. Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety

### Imaging and Images

As used herein, the term “imaging” refers to any method of obtaining an image, e.g., a microscope image of a biological sample. For example, images include bright-field images, which are transmission microscopy images where broad-spectrum, white light is placed on one side of the sample mounted on a substrate and the camera objective is placed on the other side and the sample itself filters the light in order to generate colors or grayscale intensity images. In some embodiments, the term “image” and “two-dimensional spatial representation” are interchangeable. For instance, in some embodiments, a two-dimensional spatial representation refers to an image of a biological sample. In some embodiments, a two-dimensional spatial arrangement comprises two-dimensional positions indicating the location of analyte data (e.g., for each entity in a plurality of entities). In some embodiments, a two-dimensional spatial arrangement of analyte data (e.g., for a plurality of entities) is obtained by aligning the data for the plurality of entities with an image of the biological sample. In some embodiments, a two-dimensional spatial representation refers to an image of a biological sample that is overlaid onto analyte data (e.g., for a plurality of entities).

In some embodiments, an image is acquired using transmission light microscopy (e.g., bright field transmission light microscopy, dark field transmission light microscopy, oblique illumination transmission light microscopy, dispersion staining transmission light microscopy, phase contrast transmission light microscopy, differential interference contrast transmission light microscopy, emission imaging, etc.). See, for example, Methods in Molecular Biology, 2018, Light Microscopy Method and Protocols, Markaki and Harz eds., Humana Press, New York, New York, ISBN-13: 978-1493983056, which is hereby incorporated by reference.

In some embodiments, in addition to or instead of bright-field imaging, emission imaging, such as fluorescence imaging is used. In emission imaging approaches, the sample on the substrate is exposed to light of a specific narrow band (first wavelength band) of light and the light that is re-emitted from the sample at a slightly different wavelength (second wavelength band) is measured. This absorption and re-emission is due to the presence of a fluorophore that is sensitive to the excitation used and can be either a natural property of the sample or an agent the sample has been exposed to in preparation for the imaging. As an example, in an immunofluorescence experiment, an antibody that binds to a certain protein or class of proteins, and that is labeled with a certain fluorophore, is added to the sample. The locations on the sample that include the protein or class of proteins will then emit the second wavelength band. In some implementations, multiple antibodies with multiple fluorophores can be used to label multiple proteins in the sample. Each such fluorophore undergoes excitation with a different wavelength of light and further emits a different unique wavelength of light. In order to spatially resolve each of the different emitted wavelengths of light, the sample is subjected to the different wavelengths of light that will excite the multiple fluorophores on a serial basis and images for each of these light exposures is saved as an image thus generating a plurality of images. For instance, the image is subjected to a first wavelength that excites a first fluorophore to emit at a second wavelength and a first image of the sample is taken while the sample is being exposed to the first wavelength. The exposure of the sample to the first wavelength is discontinued and the sample is exposed to a third wavelength (different from the first wavelength) that excites a second fluorophore at a fourth wavelength (different from the second wavelength) and a second image of the sample is taken while the sample is being exposed to the third wavelength. Such a process is repeated for each different fluorophore in the multiple fluorophores (e.g., two or more fluorophores, three or more fluorophores, four or more fluorophores, five or more fluorophores). In this way, a series of images of the tissue, each depicting the spatial arrangement of some different parameter such as a particular protein or protein class, is obtained. In some embodiments, more than one fluorophore is imaged at the same time. In such an approach a combination of excitation wavelengths are used, each for one of the more than one fluorophores, and a single image is collected.

In some embodiments, each of the images in a set of images for a biological sample is acquired by using a different bandpass filter that blocks out light other than a particular wavelength or set of wavelengths. In some embodiments, the set of images of a projection are images created using fluorescence imaging, for example, by making use of various immunohistochemistry (IHC) probes that excite at various different wavelengths. See, for example, Day and Davidson, 2014, “The Fluorescent Protein Revolution (In Cellular and Clinical Imaging),” CRC Press, Taylor & Francis Group, Boca Raton, Florida; “Quantitative Imaging in Cell Biology,” Methods in Cell Biology 123, 2014, Wilson and Tran, eds.; Advanced Fluorescence Reporters in Chemistry and Biology II: Molecular Constructions, Polymers and Nanoparticles (Springer Series on Fluorescence), 2010, Demchenko, ed., Springer-Verlag, Berlin, Germany; Fluorescence Spectroscopy and Microscopy: Methods and Protocols (Methods in Molecular Biology) 2014^{th} Edition, 2014, Engelborghs and Visser, eds., HumanPress; Maniatis, 2019, “Spatiotemporal Dynamics of Molecular Pathology in Amyotrophic Lateral Sclerosis,” Science 364(6435), pp. 89-93, each of which is hereby incorporated by reference for their disclosure on fluorescence imaging.

In some embodiments, an image is acquired using Epi-illumination mode, where both the illumination and detection are performed from one side of the sample. In some embodiments, an image is acquired using confocal microscopy, two-photon imaging, wide-field multiphoton microscopy, single plane illumination microscopy or light sheet fluorescence microscopy. See, for example, Adaptive Optics for Biological Imaging, 2013, Kubby ed., CRC Press, Boca Raton, Florida; Confocal and Two-Photon Microscopy: Foundations, Applications and Advances, 2002, Diaspro ed., Wiley Liss, New York, New York; and Handbook of Biological Confocal Microscopy, 2002, Pawley ed., Springer Science + Business Media, LLC, New York, New York, each of which is hereby incorporated by reference.

In some embodiments, each respective image in a plurality of images corresponds to a different biological sample in a plurality of biological samples.

In some embodiments, an image is a grayscale image. To differentiate such grayscaled images, in some embodiments, each image in a plurality of images are assigned a color (shades of red, shades of blue, etc.). In some implementations, each image is then combined into one composite color image for viewing. This allows for the spatial analysis of analytes (e.g., spatial proteomics, spatial transcriptomics, etc.) in the sample. In some embodiments, spatial analysis of one type of analyte is performed independently of any other analysis. In some embodiments, spatial analysis is performed together for a plurality of types of analytes.

In some embodiments, a biological sample is stained prior to imaging using, e.g., fluorescent, radioactive, chemiluminescent, calorimetric, or colorimetric detectable markers. In some embodiments, the biological sample is stained using live/dead stain (e.g., trypan blue). In some embodiments, the biological sample is stained with Haemotoxylin and Eosin, a Periodic acid-Schiff reaction stain (stains carbohydrates and carbohydrate rich macromolecules a deep red color), a Masson’s trichrome stain (nuclei and other basophilic structures are stained blue, cytoplasm, muscle, erythrocytes and keratin are stained bright-red, collagen is stained green or blue, depending on which variant of the technique is used), an Alcian blue stain (a mucin stain that stains certain types of mucin blue, and stains cartilage blue and can be used with H&E, and with van Gieson stains), a van Gieson stain (stains collagen red, nuclei blue, and erythrocytes and cytoplasm yellow, and can be combined with an elastin stain that stains elastin blue/black), a reticulin stain, an Azan stain, a Giemsa stain, a Toluidine blue stain, an isamin blue/eosin stain, a Nissl and methylene blue stain, a sudan black and osmium stain, and/or an immunofluorescence (IF) stain (e.g., an immunofluorescence label conjugated to an antibody).

In some embodiments, an image is in any file format including but not limited to JPEG/JFIF, TIFF, Exif, PDF, EPS, GIF, BMP, PNG, PPM, PGM, PBM, PNM, WebP, HDR raster formats, HEIF, BAT, BPG, DEEP, DRW, ECW, FITS, FLIF, ICO, ILBM, IMG, PAM, PCX, PGF, JPEG XR, Layered Image File Format, PLBM, SGI, SID, CD5, CPT, PSD, PSP, XCF, PDN, CGM, SVG, PostScript, PCT, WMF, EMF, SWF, XAML, and/or RAW.

In some embodiments, an image is obtained in any electronic color mode, including but not limited to grayscale, bitmap, indexed, RGB, CMYK, HSV, lab color, duotone, and/or multichannel. In some embodiments, the image is manipulated (e.g., stitched, compressed and/or flattened). In some embodiments, an image has a file size that is between 1 KB and 1 MB, between 1 MB and 0.5 GB, between 0.5 GB and 5 GB, between 5 GB and 10 GB, between 0.5 GB and 10 GB, between 0.5 GB and 25 GB, or greater than 25 GB. In some embodiments, the image includes between 1 million and 25 million pixels. In some embodiments, a respective image corresponds to a two-dimensional spatial arrangement of a plurality of entities, where each entity is represented by five or more, ten or more, 100 or more, or 1000 or more contiguous pixels in the respective image. In some embodiments, each entity is represented by between 1000 and 250,000 contiguous pixels in the respective image.

In some embodiments, an image is represented as an array (e.g., matrix) comprising a plurality of pixels, such that the location of each respective pixel in the plurality of pixels in the array (e.g., matrix) corresponds to its original location in the image. In some embodiments, an image is represented as a vector comprising a plurality of pixels, such that each respective pixel in the plurality of pixels in the vector comprises spatial information corresponding to its original location in the image.

### Nucleic Acid and Nucleotide

As used herein, the terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

### Partition

The term “partition,” as used herein, generally, refers to a space or volume that may be suitable to contain one or more species or conduct one or more reactions. In some embodiments, a partition is a physical compartment, such as a droplet or well. In some embodiments, the partition can isolate space or volume from another space or volume. In some embodiments, a partition (e.g., a droplet) is a first phase (e.g., aqueous phase) in a second phase (e.g., oil) immiscible with the first phase. For instance, the droplet may be a first phase in a second phase that does not phase separate from the first phase, such as, for example, a capsule or liposome in an aqueous phase. A partition may comprise one or more other (inner) partitions. In some cases, a partition is a virtual compartment that can be defined and identified by an index (e.g., indexed libraries) across multiple and/or remote physical compartments. For example, a physical compartment may comprise a plurality of virtual compartments.

### Region of Interest

As used herein, the term “region of interest” generally refers to a region or area within a biological sample that is selected for specific analysis (e.g., a region in a biological sample that has morphological features of interest). A biological sample can have regions that show morphological feature(s) that may indicate the presence of disease or the development of a disease phenotype. For example, morphological features at a specific site within a tumor biopsy sample can indicate the aggressiveness, therapeutic resistance, metastatic potential, migration, stage, diagnosis, and/or prognosis of cancer in a subject. A change in the morphological features at a specific site within a tumor biopsy sample often correlate with a change in the level or expression of an analyte in a cell within the specific site, which can, in turn, be used to provide information regarding the aggressiveness, therapeutic resistance, metastatic potential, migration, stage, diagnosis, and/or prognosis of cancer in a subject. A region of interest in a biological sample can be used to analyze a specific area of interest within a biological sample, and thereby, focus experimentation and data gathering to a specific region of a biological sample (rather than an entire biological sample). This results in increased time efficiency of the analysis of a biological sample.

A region of interest can be identified in a biological sample using a variety of different techniques, e.g., expansion microscopy, bright field microscopy, dark field microscopy, phase contrast microscopy, electron microscopy, fluorescence microscopy, reflection microscopy, interference microscopy, and confocal microscopy, and combinations thereof. For example, the staining and imaging of a biological sample can be performed to identify a region of interest. In some examples, the region of interest can correspond to a specific structure of cytoarchitecture. In some embodiments, a biological sample can be stained prior to visualization to provide contrast between the different regions of the biological sample. The type of stain can be chosen depending on the type of biological sample and the region of the cells to be stained. In some embodiments, more than one stain can be used to visualize different aspects of the biological sample, e.g., different regions of the sample, specific cell structures (e.g., organelles), or different cell types. In other embodiments, the biological sample can be visualized or imaged without staining the biological sample.

In some examples, a region of interest can be removed from a biological sample and then the region of interest can be contacted to the substrate and/or array (e.g., as described herein). A region of interest can be removed from a biological sample using microsurgery, laser capture microdissection, chunking, a microtome, dicing, trypsinization, labelling, and/or fluorescence-assisted cell sorting.

### Subject

As used herein, the term “subject” refers to an animal, such as a mammal (e.g., human or a non-human simian), avian (e.g., bird), or other organism, such as a plant. Examples of subjects include, but are not limited to, a mammal such as a rodent, mouse, rat, rabbit, guinea pig, ungulate, horse, sheep, pig, goat, cow, cat, dog, primate (e.g., human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum.

### Substrates

As used herein, a “substrate” refers to a support that is insoluble in aqueous liquid and that allows for positioning of biological samples, analytes, capture spots, and/or capture probes on the substrate. For instance, a substrate can be any surface onto which a sample and/or capture probes can be affixed (e.g., a chip, solid array, a bead, a slide, a coverslip, etc.). For the spatial analytical methods described in this section, a substrate is used to provide support to a biological sample, particularly, for example, a thin tissue section. In addition, in some embodiments, a substrate (e.g., the same substrate or a different substrate) functions as a support for direct or indirect attachment of capture probes to capture spots of the array.

A wide variety of different substrates can be used for the foregoing purposes. In general, a substrate can be any suitable support material. Exemplary substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides, etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene and polycarbonate.

The substrate can also correspond to a flow cell. Flow cells can be formed of any of the foregoing materials, and can include channels that permit reagents, solvents, capture spots, and molecules to pass through the flow cell.

The substrate can generally have any suitable form or format. For example, the substrate can be flat, curved, e.g., convexly or concavely curved towards the area where the interaction between a biological sample, e.g., tissue sample, and the substrate takes place. In some embodiments, the substrate is a flat, e.g., planar, chip or slide. The substrate can contain one or more patterned surfaces within the substrate (e.g., channels, wells, projections, ridges, divots, etc.). A substrate can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure can be any appropriate type of support having a flat surface (e.g., a chip or a slide such as a microscope slide).

In some embodiments, a substrate includes one or more markings on a surface of the substrate, e.g., to provide guidance for correlating spatial information with the characterization of the analyte of interest. For example, a substrate can be marked with a grid of lines (e.g., to allow the size of objects seen under magnification to be easily estimated and/or to provide reference areas for counting objects). In some embodiments, fiducials (e.g., fiducial markers, fiducial spots, or fiducial patterns) can be included on the substrate. Fiducials can be made using techniques including, but not limited to, printing, sand-blasting, and depositing on the surface. In some embodiments, the substrate (e.g., or a bead or a capture spot on an array) includes a plurality of oligonucleotide molecules (e.g., capture probes). In some embodiments, the substrate includes tens to hundreds of thousands or millions of individual oligonucleotide molecules (e.g., at least about 10,000, 50,000, 100,000, 500,000, 1,000,000, 10,000,000, 100,000,000, 1,000,000,000 or 10,000,000,000 oligonucleotide molecules). In some embodiments, a substrate can include a substrate identifier, such as a serial number.

Further examples of substrates, including for example fiducial markers on such substrates, are disclosed in PCT Publication No. 202020176788A1, entitled “Profiling of biological analytes with spatially barcoded oligonucleotide arrays”; in U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat, Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety

### Spatial Analyte Data

As used herein, “spatial analyte data” refers to any data measured, either directly, from the capture of analytes on capture probes, or indirectly, through intermediate agents disclosed herein that bind to analytes in a sample, e.g., connected probes disclosed herein, analyte capture agents or portions thereof (such as, e.g., analyte binding moieties and their associated analyte binding moiety barcodes). Spatial analyte data thus may, in some aspects, include two different labels from two different classes of barcodes. One class of barcode identifies the analyte, while the other class of barcodes identifies the specific capture probe in which an analyte was detected.

### (B) Methods for Single Cell Analysis of Analytes

In the embodiments of the present disclosure, techniques for analyzing biological samples are provided. The techniques involve acquiring a sample (e.g., a tumor biopsy, a sample of any tissue, body fluid, etc.) and processing the sample to acquire data from each cell in the sample for computational analysis. Each cell in the sample is barcoded, at a minimum, as discussed below.

Generally, in some embodiments, microfluidic partitions are used to partition very small numbers of entities (e.g., cells, groups of analytes, mRNA molecules, etc.) and to barcode those partitions. In some such embodiments, where discrete attribute values are measured from single cells, the microfluidic partitions are used to capture individual cells within each microfluidic droplet and then pools of single barcodes within each of those droplets are used to tag all of the contents of a given cell. For example, in some embodiments, a pool of ~ 750,000 barcodes is sampled to separately index each entity’s transcriptome by partitioning thousands of entities into nanoliter-scale Gel Bead-In-EMulsions (GEMs), where all generated cDNA share a common barcode. Libraries are generated and sequenced from the cDNA and the barcodes are used to associate individual reads back to the individual partitions. In other words, each respective droplet (GEM) is assigned its own barcode and all the contents (e.g., cells, analytes, etc.) in a respective droplet are tagged with the barcode unique to the respective droplet. In some embodiments, such droplets are formed as described in Zheng et al., 2016, Nat Biotechnol. 34(3): 303-311; or in See the Chromium, Single Cell 3’ Reagent Kits v2. User Guide, 2017, 10X Genomics, Pleasanton, California, Rev. B, page, 2, each of which is hereby incorporated by reference. In some alternative embodiments, equivalent 5’ chemistry is used rather than the 3’ chemistry disclosed in these references.

In some embodiments there are tens, hundreds, thousands, tens of thousands, or hundreds of thousands of such microfluidic droplets. In some such embodiments, at least seventy percent, at least eighty percent, at least ninety percent, at least ninety percent, at least ninety-five percent, at least ninety-eight percent, or at least ninety-nine percent of the respective microfluidic droplets contain either no second entity 126 (e.g., 1 entity per droplet) or a single second entity 126 (e.g., at most 2 entities per droplet) while the remainder of the microfluidic droplets contain two or more second entities 126. In other words, to achieve single entity resolution, the entities are delivered at a limiting dilution, such that the majority (-90-99%) of generated nanoliter-scale gel bead-in-emulsions (GEMs) contains no second entity, while the remainder largely contain a single second entity. See the Chromium, Single Cell 3' Reagent Kits v2. User Guide, 2017, 10X Genomics, Pleasanton, California, Rev. B, page, 2, which is hereby incorporated by reference. In some alternative embodiments, equivalent 5’ chemistry is used rather than the 3’ chemistry disclosed in this reference.

Within an individual droplet, gel bead dissolution releases the amplification primer into the partitioned solution. In some embodiments, upon dissolution of the single second entity 3’ Gel Bead in a GEM, primers containing (i) an Illumina R1 sequence (read 1 sequencing primer), (ii) a 16 bp 10x Barcode, (iii) a 10 bp Unique Molecular Identifier (UMI) and (iv) a polydT primer sequence are released and mixed with cell lysate and Master Mix. Incubation of the GEMs then produces barcoded, full-length cDNA from poly-adenylated mRNA. After incubation, the GEMs are broken, and the pooled fractions are recovered. See the Chromium, Single Cell 3' Reagent Kits v2. User Guide, 2017, 10X Genomics, Pleasanton, California, Rev. B, page, 2, which is hereby incorporated by reference. In some such embodiments, silane magnetic beads are used to remove leftover biochemical reagents and primers from the post GEM reaction mixture. Full-length, barcoded cDNA is then amplified by PCR to generate sufficient mass for library construction.

In this way, the discrete attribute values (e.g., of analytes) derived from a first respective entity 126 (e.g., a first cell) can be distinguished from the discrete attribute values (e.g., of analytes) derived from a second respective entity 126 (e.g., a second cell) based on the unique barcode. This contrasts to bulk sequencing techniques in which all the entities (e.g., cells) are pooled together and the measurement profile is that of the plurality of entities (e.g., the plurality of cells) without the ability to distinguish the measurement signal between individual entities. An example of such measurement techniques is disclosed in United States Patent Application 2015/0376609, which is hereby incorporated by reference. As such, in some embodiments, each discrete attribute value for a respective entity in the plurality of entities is barcoded with a barcode that is unique to the respective entity. In some embodiments, the discrete attribute value 124 of each respective analyte for a respective entity 126 is determined after the respective entity 126 has been separated from all the other entities in the plurality of entities into its own microfluidic partition.

The acquired data is stored, for example, in specific data structure(s), for processing by one or more processors (or processing cores) that are configured to access the data structures and to perform computational analysis such that biologically meaningful patterns within the sample are detected. The computational analysis and associated computer-generated visualization of results of the computational analysis on a graphical user interface allow for the observation of properties of the sample that would not otherwise be detectable. In particular, in some embodiments, each cell of the sample is subjected to analysis and characteristics of each cell within the sample are obtained such that it becomes possible to characterize the sample based on differentiation among different types of cells in the sample. For example, the clustering analysis, as well as other techniques of data analysis described above, reveal distributions of cell populations and sub-populations within a sample that would not be otherwise discernable. This leads to the discovery of novel cell types, or to the novel discovery of relationships between (A) aspects of the cellular phenotypes, such as genome (e.g., genomic rearrangements, structural variants, copy number variants, single nucleotide polymorphisms, loss of heterozygosity, rare variants), epigenome (e.g., DNA methylation, histone modification, chromatin assembly, protein binding), transcriptome (e.g., gene expression, alternative splicing, non-coding RNAs, small RNAs), proteome (e.g., protein abundance, protein-protein interactions, cytokine screening), metabolome (e.g., absence, presence, or amount of small molecules, drugs, metabolites, and lipids), and/or phenome (e.g., functional genomics, genetics screens, morphology), and (B) particular phenotypic states, such as absence or presence of a marker, participation in a biological pathway, disease state, absence or presence of a disease state, to name a few non-limiting examples. The identification of different classes of cells within the sample allows for taking an action with respect to the sample or with respect to a source of the sample. For example, depending on a distribution of cell types within a biological sample that is a tumor biopsy obtained from a subject, a specific treatment can be selected and administered to the subj ect.

Furthermore, the techniques in accordance with the described embodiments allow clustering and otherwise analyzing the discrete attribute value dataset so as to identify patterns within the dataset and thereby assign each cell to a type or class. As used in this context here, a class refers to a cell type, a disease state, a tissue type, an organ type, a species, assay conditions and/or any other feature or factor that allows for the differentiation of cells (or groups of cells) from one another. The discrete attribute value dataset includes any suitable number of cell classes of any suitable type. Moreover, as discussed above, the described techniques (including barcoding and computational analysis and visualization) provide the basis for identifying relationships between cellular phenotype and overall phenotypic state of an organism that is the source of the biological sample from which the sample was obtained that would not otherwise be discernable.

Such embodiments provide the ability to explore the heterogeneity between cells, which is one form of pattern analysis afforded by the systems and method of the present disclosure. In some embodiments where the discrete attribute value is mRNA abundance, it is possible that the mRNA abundance in the cell sample may vary vastly from cell to cell. As such, the disclosed systems and methods enable the profiling of which genes are being expressed and at what levels in each of the cells. These gene profiles, or principal components derived therefrom, can be used to cluster cells and identify populations of related cells, for instance, to identify similar gene profiles at different life cycle stages of the cell or within different types of cells, tissues, organs, and/or other sources of cell heterogeneity.

### Single Cell Sequencing Workflow

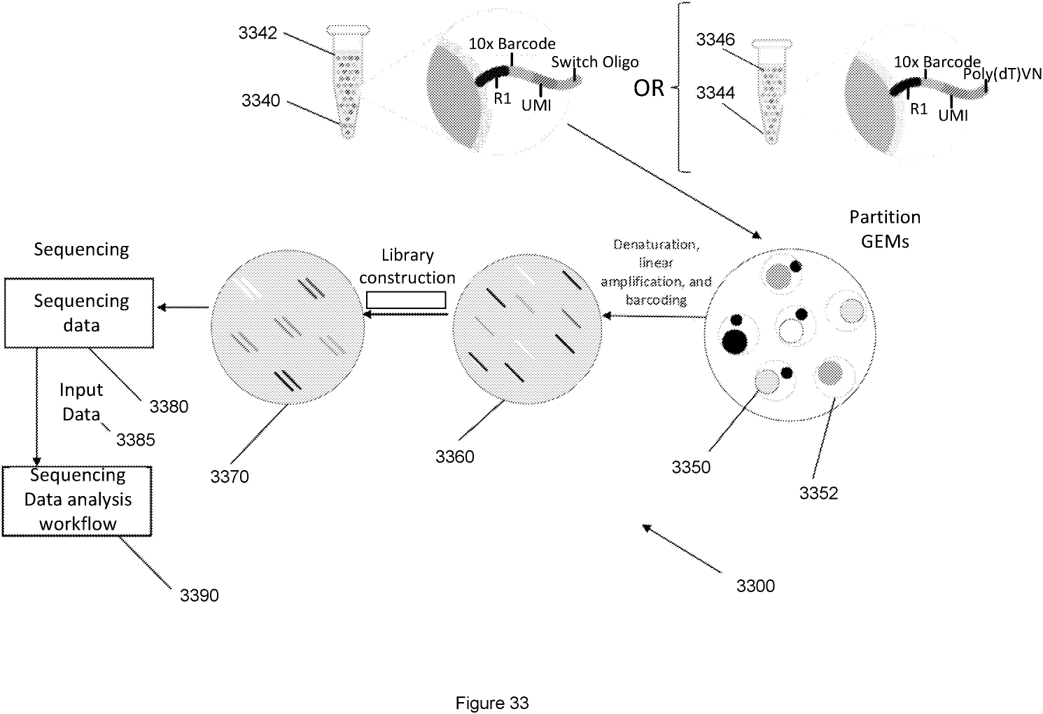

In accordance with various embodiments, a general schematic workflow is provided in FIG. 33 to illustrate a non-limiting example process for using single cell sequencing technology to generate sequencing data. Such sequencing data can be used for charactering cells and cell features in accordance with various embodiments. The workflow can include various combinations of features, including more or less features than those illustrated in FIG. 33. As such, FIG. 33 simply illustrates one example of a possible workflow.

### GEM Generation

The workflow 3300 provided in FIG. 33 begins with Gel beads-in-EMulsion (GEMs) generation. The bulk cell suspension containing the cells is mixed with a gel beads solution 3340 or 3344 containing a plurality of individually barcoded gel beads 3342 or 3346. In various embodiments, this step results in partitioning the cells into a plurality of individual GEMs 3350, each including a single cell, and a barcoded gel bead 3342 or 3346. This step also results in a plurality of GEMs 3352, each containing a barcoded gel bead 3342 or 3346 but no nuclei. Details for GEM generation, in accordance with various embodiments disclosed herein, is provided below. Further details can be found in U.S. Pat. Nos. 10,343,166 and 10,583,440, U.S. Published Application Nos. US20180179590A1, US20190367969A1, US20200002763A1, and US20200002764A1, and Published International PCT Application No. WO 2019/040637, each of which is incorporated herein by reference in its entirety.

In various embodiments, GEMs can be generated by combining barcoded gel beads, individual cells, and other reagents or a combination of biochemical reagents that may be necessary for the GEM generation process. Such reagents may include, but are not limited to, a combination of biochemical reagents (e.g., a master mix) suitable for GEM generation and partitioning oil. The barcoded gel beads 3342 or 3346 of the various embodiments herein may include a gel bead attached to oligonucleotides containing (i) an Illumina® P5 sequence (adapter sequence), (ii) a 16 nucleotide (nt) 10x Barcode, and (iii) a Read 1 (Read 1N) sequencing primer sequence. It is understood that other adapter, barcode, and sequencing primer sequences can be contemplated within the various embodiments herein.

In various embodiments, GEMS are generated by partitioning the cells using a microfluidic chip. To achieve single cell resolution per GEM, the cells can be delivered at a limiting dilution, such that the majority (e.g., ~90-99%) of the generated GEMs do not contain any cells, while the remainder of the generated GEMs largely contain a single cell.

In the methods and systems described herein, one or more labelling agents capable of binding to or otherwise coupling to one or more cell features may be used to characterize cells and/or cell features in combination with GEMs 3352. In various embodiments, the one or more labelling agents may include barcoded nucleic acid molecules, or derivatives generated therefrom, which can then be sequenced on a suitable sequencing platform to obtain datasets of sequence reads for future analysis described herein.

In various embodiments, a library of potential cell feature labelling agents may be provided associated with nucleic acid reporter molecules, e.g., where a different reporter oligonucleotide sequence is associated with each labelling agent capable of binding to a specific cell feature. The cell feature labelling agents may comprise a functional sequence that can be configured to hybridize to a commentary sequence present on a nucleotide acid barcode molecule on individually barcoded gel beads 3342 or 3346.

In some aspects, different members of the library may be characterized by the presence of a different oligonucleotide sequence label, e.g., an antibody capable of binding to a first type of protein may have associated with it a first known reporter oligonucleotide sequence, while an antibody capable of binding to a second protein (i.e., different than the first protein) may have a different known reporter oligonucleotide sequence associated with it.

Prior to partitioning, the cells may be incubated with the library of labelling agents, that may represent labelling agents to a broad panel of different cell features, e.g., receptors, proteins, etc., and which include their associated reporter oligonucleotides. Unbound labelling agents may be washed from the cells, and the cells may then be co-partitioned (e.g., into droplets or wells) along with partition-specific barcode oligonucleotides (e.g., attached to a bead, such as a gel bead). As a result, the partitions may include the cell or cells, as well as the bound labelling agents and their known, associated reporter oligonucleotides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. In this way, different samples or groups can be independently processed and subsequently combined for pooled analysis (e.g., partition-based barcoding as described elsewhere herein). See, e.g., U.S. Pat. Pub. 20190323088, which is hereby incorporated by reference its entirety.

### Barcoding RNA Molecules of Fragments

The workflow 3300 provided in FIG. 33 further includes lysing the cells and barcoding the RNA molecules or fragments for producing a plurality of uniquely barcoded single-stranded nucleic acid molecules or fragments. Upon generation of the GEMs 3350, the gel beads 3342 or 3346 can be dissolved releasing the various oligonucleotides of the embodiments described above, which are then mixed with the RNA molecules or fragments resulting in a plurality of uniquely barcoded single-stranded nucleic acid molecules or fragments 3360 following a nucleic acid extension reaction, e.g., reverse transcription of mRNA to cDNA, within the GEMs 3350. Detail related to generation of the plurality of uniquely barcoded single-stranded nucleic acid molecules or fragments 3360, in accordance with various embodiments disclosed herein, is provided below.

In various embodiments, upon generation of the GEMs 3350, the gel beads 3342 or 3346 can be dissolved, and oligonucleotides of the various embodiments disclosed herein, containing a capture sequence, e.g., a poly(dT) sequence or a template switch oligonucleotide (TSO) sequence, a unique molecular identifier (UMI), a unique 10x Barcode, and a Read 1 sequencing primer sequence can be released and mixed with the RNA molecules or fragments and other reagents or a combination of biochemical reagents (e.g., a master mix necessary for the nucleic acid extension process). Denaturation and a nucleic acid extension reaction, e.g., reverse transcription, within the GEMs can then be performed to produce a plurality of uniquely barcoded single-stranded nucleic acid molecules or fragments 3360. In various embodiments herein, the plurality of uniquely barcoded single-stranded nucleic acid molecules or fragments 3360 can be 10x barcoded single-stranded nucleic acid molecules or fragments. In one non-limiting example of the various embodiments herein, a pool of ~750,000, 10x barcodes are utilized to uniquely index and barcode nucleic acid molecules derived from the RNA molecules or fragments of each individual cell.

Accordingly, the in-GEM barcoded nucleic acid products of the various embodiments herein can include a plurality of 10x barcoded single-stranded nucleic acid molecules or fragments that can be subsequently removed from the GEM environment and amplified for library construction, including the addition of adaptor sequences for downstream sequencing. In one non-limiting example of the various embodiments herein, each such in-GEM 10x barcoded single-stranded nucleic acid molecule or fragment can include a unique molecular identifier (UMI), a unique 10x barcode, a Read 1 sequencing primer sequence, and a fragment or insert derived from an RNA fragment of the cell, e.g., cDNA from an mRNA via reverse transcription. Additional adaptor sequence may be subsequently added to the in-GEM barcoded nucleic acid molecules after the GEMs are broken.

In various embodiments, after the in-GEM barcoding process, the GEMs 3350 are broken and pooled barcoded nucleic acid molecules or fragments are recovered. The 10x barcoded nucleic acid molecules or fragments are released from the droplets, i.e., the GEMs 3350, and processed in bulk to complete library preparation for sequencing, as described in detail below. In various embodiments, following the amplification process, leftover biochemical reagents can be removed from the post-GEM reaction mixture. In one embodiment of the disclosure, silane magnetic beads can be used to remove leftover biochemical reagents. Additionally, in accordance with embodiments herein, the unused barcodes from the sample can be eliminated, for example, by Solid Phase Reversible Immobilization (SPRI) beads.

### Library Construction

The workflow 3300 provided in FIG. 33 further includes a library construction step. In the library construction step of workflow 3300, a library 3370 containing a plurality of double-stranded DNA molecules or fragments are generated. These double-stranded DNA molecules or fragments can be utilized for completing the subsequent sequencing step. Detail related to the library construction, in accordance with various embodiments disclosed herein, is provided below.

In accordance with various embodiments disclosed herein, an Illumina® P7 sequence and P5 sequence (adapter sequences), a Read 2 (Read 2N) sequencing primer sequence, and a sample index (SI) sequence(s) (e.g., i7 and/or i5) can be added during the library construction step via PCR to generate the library 3370, which contains a plurality of double stranded DNA fragments. In accordance with various embodiments herein, the sample index sequences can each comprise of one or more oligonucleotides. In one embodiment, the sample index sequences can each comprise of four to eight or more oligonucleotides. In various embodiments, when analyzing the single cell sequencing data for a given sample, the reads associated with all four of the oligonucleotides in the sample index can be combined for identification of a sample. Accordingly, in one non-limiting example, the final single cell gene expression analysis sequencing libraries contain sequencer compatible double-stranded DNA fragments containing the P5 and P7 sequences used in Illumina® bridge amplification, sample index (SI) sequence(s) (e.g., i7 and/or i5), a unique 10x barcode sequence, and Read 1 and Read 2 sequencing primer sequences.

Various embodiments of single cell sequencing technology within the disclosure can at least include platforms such as One Sample, One GEM Well, One Flowcell; One Sample, One GEM well, Multiple Flowcells; One Sample, Multiple GEM Wells, One Flowcell; Multiple Samples, Multiple GEM Wells, One Flowcell; and Multiple Samples, Multiple GEM Wells, Multiple Flowcells platform. Accordingly, various embodiments within the disclosure can include sequence dataset from one or more samples, samples from one or more donors, and multiple libraries from one or more donors.

The workflow 3300 provided in FIG. 33 further includes a sequencing step. In this step, the library 3370 can be sequenced to generate a plurality of sequencing data 3380. The fully constructed library 3370 can be sequenced according to a suitable sequencing technology, such as a next-generation sequencing protocol, to generate the sequencing data 3380. In various embodiments, the next-generation sequencing protocol utilizes the llumina® sequencer for generating the sequencing data. It is understood that other next-generation sequencing protocols, platforms, and sequencers such as, e.g., MiSeq™, NextSeq™ 500/550 (High Output), HiSeq 2500™ (Rapid Run), HiSeq™ 3000/4000, and NovaSeq™, can be also used with various embodiments herein.

### Sequencing Data Input and Data Analysis Workflow

The workflow 3300 provided in FIG. 33 further includes a sequencing data analysis workflow 3390. With the sequencing data 3380 in hand, the data can then be output, as desired, and used as an input data 3385 for the downstream sequencing data analysis workflow 3390, in accordance with various embodiments herein. Sequencing the single cell libraries produces standard output sequences (also referred to as the “sequencing data”, “sequence data”, or the “sequence output data”) that can then be used as the input data 3385, in accordance with various embodiments herein. For instance, in some embodiments, the sequencing data comprises a plurality of discrete attribute values that are stored in a discrete attribute value dataset. The sequence data contains sequenced fragments (also interchangeably referred to as “fragment sequence reads”, “sequencing reads” or “reads”), which in various embodiments include RNA sequences of the RNA fragments containing the associated 10x barcode sequences, adapter sequences, and primer oligo sequences.

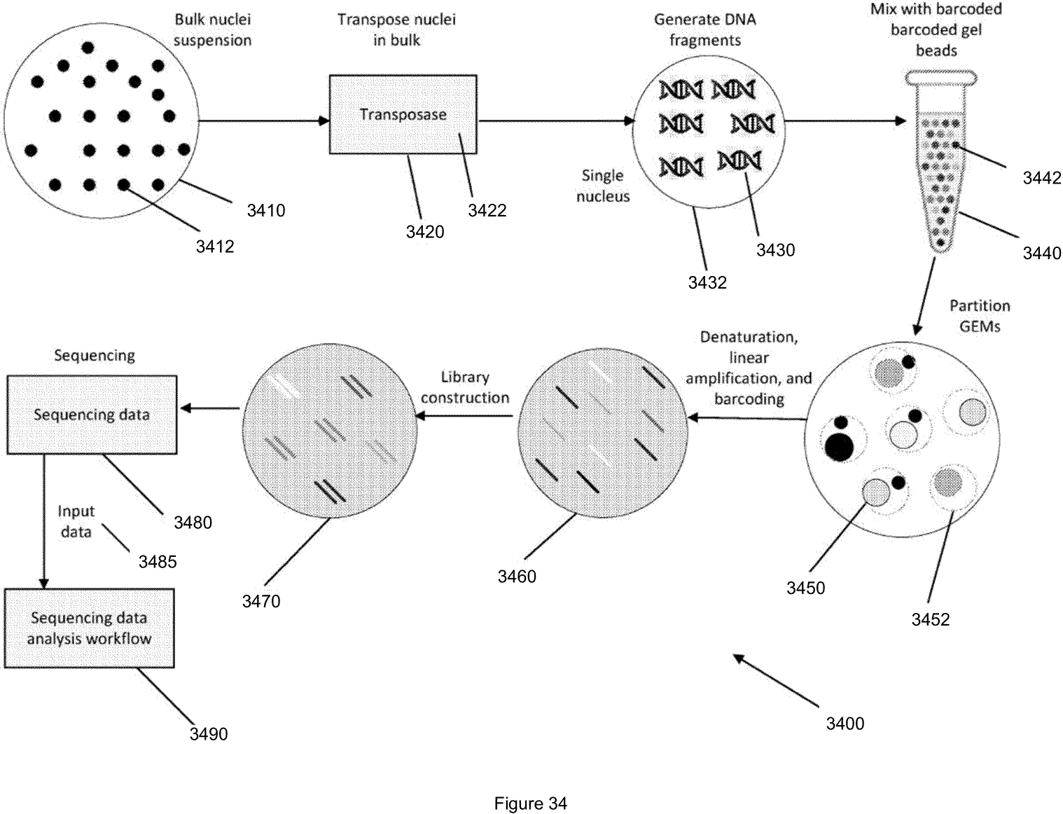

With reference to FIG. 34, another exemplary workflow 3400 includes using single cell Assay for Transposase Accessible Chromatin (ATAC) sequencing technology to generate sequencing data. Such sequencing data can be used for identifying genome-wide differential accessibility of gene regulatory elements in accordance with various embodiments. In some embodiments, the workflow includes obtaining a bulk nuclei suspension 3410 from a sample comprising a plurality of individual nuclei 3412. In various embodiments, obtaining a bulk nuclei suspension can include isolating nuclei in bulk from a sample. It is understood that one problem with generating ATAC sequencing datasets, is that the dataset may contain a large percentage of read sequences (also referred to as reads) from mitochondrial DNA. Various methods can be employed for ensuring low mitochondrial reads from samples and high quality nuclei sequencing data. Accordingly, in some embodiments, preparation of the bulk nuclei suspension can include carefully extracting nuclei from cells, while ensuring the mitochondria stays intact. In some embodiments, the workflow further includes transposing the bulk nuclei suspension and generating adapter-tagged DNA fragments. The bulk nuclei suspension 3410 is incubated with a transposition mix 3420 containing Transposase 3422. Upon incubation, the Transposase 3422 enters individual nuclei 3412 and preferentially fragments the DNA in open regions of a chromatin to generate a plurality of adapter-tagged DNA fragments 3430 inside individual transposed nucleus 3432. Using the adapter-tagged DNA fragments 3430, the bulk nuclei suspension containing individual transposed nuclei 3432 is mixed with a gel beads solution 3440 containing a plurality of individually barcoded gel beads 3442. In various embodiments, this step results in partitioning the nuclei into a plurality of individual GEMs 3450, each including a single transposed nucleus 3432 that contains a plurality of adapter-tagged DNA fragments 3430, and a barcoded gel bead 3442. This step also results in a plurality of GEMS 3452, each containing a barcoded gel bead 3442 but no nuclei. Details related to GEM generation, in accordance with various embodiments disclosed herein, are provided above with reference to FIG. 33.

FIG. 34 further illustrates barcoding the adapter-tagged DNA fragments 3430 for producing a plurality of uniquely barcoded single-stranded DNA fragments 3460 and generating a library 3470 containing a plurality of double-stranded DNA fragments. The workflow 3400 further includes a sequencing step, in which the library 3470 can be sequenced to generate a plurality of sequencing data 3480. The data can then be output, as desired, and used as an input data 3485 for the downstream sequencing data analysis 3490. Details related to barcoding, library preparation, sequencing, and data analysis, in accordance with various embodiments disclosed herein, are provided above with reference to FIG. 33.

The various embodiments, systems and methods within the disclosure further include processing and inputting the sequence data. A compatible format of the sequencing data of the various embodiments herein can be a FASTQ file. Other file formats for inputting the sequence data is also contemplated within the disclosure herein. Various software tools within the embodiments herein can be employed for processing and inputting the sequencing output data into input files for the downstream data analysis workflow. It is understood that various systems and methods with the embodiments herein are contemplated that can be employed to independently analyze the inputted single cell sequencing data for studying cells and cell features in accordance with various embodiments.

Additional embodiments for single cell analysis of analytes are further disclosed in, for example, PCT Application No. PCT/US21/42992, entitled “Systems and Methods for Detecting and Removing Aggregates for Calling Cell-Associated Barcodes,” filed Jul. 23, 2021; U.S. Patent Application No. 17/231,972, entitled “Systems and Methods for Identifying Differential Accessibility of Gene Regulatory Elements at Single Cell Resolution,” filed Apr. 15, 2021; U.S. Patent Application No. 17/175,577, entitled “SYSTEMS AND METHODS FOR JOINT INTERACTIVE VISUALIZATION OF GENE EXPRESSION AND DNA CHROMATIN ACCESSIBILITY,” filed Feb. 12, 2021; U.S. Patent Application No. 16/442,800, entitled, “ Systems and Methods for Visualizing a Pattern in a Dataset,” filed Jun. 17, 2019; and U.S. Patent Application No. 17/239,555, entitled “Capturing Targeted Genetic Targets Using a Hybridization/Capture Approach,” filed Apr. 24, 2021, each of which is hereby incorporated herein by reference in its entirety.

### (C) Methods for Spatial Analysis of Analytes

Array-based spatial analysis methods involve the transfer of one or more analytes from a biological sample to an array of capture spots on a substrate, each of which is associated with a unique spatial location on the array. Subsequent analysis of the transferred analytes includes determining the identity of the analytes and the spatial location of each analyte within the sample. The spatial location of each analyte within the sample is determined based on the capture spot to which each analyte is bound in the array, and the capture spot’s relative spatial location within the array.

There are at least two general methods to associate a spatial barcode with one or more neighboring cells, such that the spatial barcode identifies the one or more cells, and/or contents of the one or more cells, as associated with a particular spatial location. One general method is to promote analytes out of a cell and towards the spatially-barcoded array. In an exemplary embodiment of this general method, the spatially-barcoded array populated with capture probes (as described further herein) is contacted with a sample, and the sample is permeabilized, allowing the target analyte to migrate away from the sample and toward the array. The target analyte interacts with a capture probe on the spatially-barcoded array. Once the target analyte hybridizes/is bound to the capture probe, the sample is optionally removed from the array and the capture probes are analyzed in order to obtain spatially-resolved analyte information.

Another general method is to cleave the spatially-barcoded capture probes from an array and promote the spatially-barcoded capture probes towards and/or into or onto the sample. In an exemplary embodiment of this general method, the spatially-barcoded array populated with capture probes (as described further herein) can be contacted with a sample. The spatially-barcoded capture probes are cleaved and then interact with cells within the provided sample. The interaction can be a covalent or non-covalent cell-surface interaction. The interaction can be an intracellular interaction facilitated by a delivery system or a cell penetration peptide. Once the spatially-barcoded capture probe is associated with a particular cell, the sample can be optionally removed for analysis. The sample can be optionally dissociated before analysis. Once the tagged cell is associated with the spatially-barcoded capture probe, the capture probes can be analyzed to obtain spatially-resolved information about the tagged cell.

Other exemplary workflows that include preparing a sample on a spatially-barcoded array may include placing the sample on a substrate (e.g., chip, slide, etc.), fixing the sample, and/or staining the sample for imaging. The sample (stained or not stained) is then imaged on the array using bright-field (to image the sample, e.g., using a hematoxylin and eosin stain) or fluorescence (to image capture spots) and/or emission imaging modalities. In some embodiments where the sample is analyzed with transcriptomics, along with the bright-field and/or emission imaging (e.g., fluorescence imaging), target analytes are released from the sample and capture probes forming a spatially-barcoded array hybridize or bind the released target analytes. The sample can be optionally removed from the array and the capture probes can be optionally cleaved from the array. The sample and array are then optionally imaged a second time in both modalities while the analytes are reverse transcribed into cDNA, and an amplicon library is prepared and sequenced. The images are then spatially-overlaid in order to correlate spatially-identified sample information. When the sample and array are not imaged a second time, a spot coordinate file is supplied instead. The spot coordinate file replaces the second imaging step. Further, amplicon library preparation can be performed with a unique PCR adapter and sequenced.

Another exemplary workflow utilizes a spatially-barcoded array on a substrate (e.g., chip), where spatially-barcoded capture probes are clustered at areas called capture spots. The spatially-labelled capture probes can include a cleavage domain, one or more functional sequences, a spatial barcode, a unique molecular identifier, and a capture domain. The spatially-labelled capture probes can also include a 5’ end modification for reversible attachment to the substrate. The spatially-barcoded array is contacted with a sample, and the sample is permeabilized through application of permeabilization reagents. Permeabilization reagents may be administered by placing the array/sample assembly within a bulk solution. Alternatively, permeabilization reagents may be administered to the sample via a diffusion-resistant medium and/or a physical barrier such as a lid, where the sample is sandwiched between the diffusion-resistant medium and/or barrier and the array-containing substrate. The analytes are migrated toward the spatially-barcoded capture array using any number of techniques disclosed herein. For example, analyte migration can occur using a diffusion-resistant medium lid and passive migration. As another example, analyte migration can be active migration, using an electrophoretic transfer system, for example. Once the analytes are in close proximity to the spatially-barcoded capture probes, the capture probes can hybridize or otherwise bind a target analyte. The sample can be optionally removed from the array.

The capture probes can be optionally cleaved from the array, and the captured analytes can be spatially-barcoded by performing a reverse transcriptase first strand cDNA reaction. A first strand cDNA reaction can be optionally performed using template switching oligonucleotides. For example, a template switching oligonucleotide can hybridize to a poly(C) tail added to a 3’end of the cDNA by a reverse transcriptase enzyme. Template switching is described, for example, in U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat. Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety.

The original mRNA template and template switching oligonucleotide can then be denatured from the cDNA and the spatially-barcoded capture probe can then hybridize with the cDNA and a complement of the cDNA can be generated. The first strand cDNA can then be purified and collected for downstream amplification steps. The first strand cDNA can be optionally amplified using PCR, where the forward and reverse primers flank the spatial barcode and target analyte regions of interest, generating a library associated with a particular spatial barcode. In some embodiments, the library preparation can be quantified and/or subjected to quality control to verify the success of the library preparation steps 408. In some embodiments, the cDNA comprises a sequencing by synthesis (SBS) primer sequence. The library amplicons are sequenced and analyzed to decode spatial information, with an additional library quality control (QC) step.

Yet another exemplary workflow includes where the sample is removed from the spatially-barcoded array and the spatially-barcoded capture probes are removed from the array for barcoded analyte amplification and library preparation. Another embodiment includes performing first strand synthesis using template switching oligonucleotides on the spatially-barcoded array without cleaving the capture probes. In this embodiment, sample preparation and permeabilization are performed as described elsewhere herein. Once the capture probes capture the target analyte(s), first strand cDNA created by template switching and reverse transcriptase is then denatured, and the second strand is then extended. The second strand cDNA is then denatured from the first strand cDNA, neutralized, and transferred to a tube. cDNA quantification and amplification can be performed using standard techniques discussed herein. The cDNA can then be subjected to library preparation and indexing, including fragmentation, end-repair, and a-tailing, and indexing PCR steps. The library can also be optionally tested for quality control (QC).

In some embodiments, a respective image is aligned to a plurality of probe spots on a substrate by a procedure that comprises analyzing an array of pixel values in the respective image to identify a plurality of spatial fiducials of the respective image. The spatial fiducials are aligned with a corresponding plurality of reference spatial fiducials using an alignment algorithm to obtain a transformation between the plurality of spatial fiducials of the respective image and the corresponding plurality of reference spatial fiducials. The transformation and a coordinate system corresponding to the plurality of reference spatial fiducials are then used to locate a corresponding position in the respective image of each probe spot in a plurality of probe spots.

For instance, as disclosed in U.S. Pat. Publication No. US 2021-0062272, entitled “Systems and Methods for Using the Spatial Distribution of Haplotypes to Determine a Biological Condition,” which is hereby incorporated herein by reference, in some embodiments, the biological sample is mounted onto a substrate having printed visible fiducial marks that can be identified in an obtained image, such as a brightfield image. A visualization system 119 performs alignment of the imaged fiducial pattern to the substrate. In some embodiments, a manual alignment tool in the disclosed visualization module 119 is used, where the user is guided through steps to identify these marks. In some embodiments, the visualization module 119, or a software module thereof, prepares data for the visualization module 119 using automatic segmentation of tissue images from the obtained image. See, for example, U.S Pat. Publication No. US 2021-0150707, entitled “Systems and Methods for Binary Tissue Classification,” and PCT Patent Application No. PCT/US2020/060164, entitled “Systems and Methods for Binary Tissue Classification,” filed Nov. 18, 2020, each of which is hereby incorporated by reference.

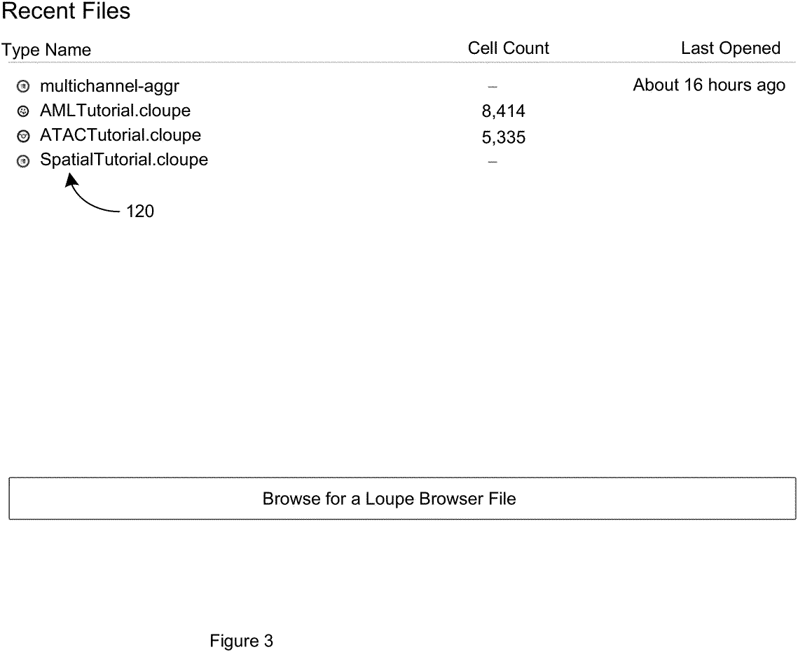

In some embodiments, spatial analysis of analyte data obtained from probe spot-based sequencing (e.g., stored in a discrete attribute value dataset) can be performed by aligning the probe spots with the image of the biological sample using the identified fiducial marks. In some embodiments, alignment is performed for a discrete attribute value dataset 120 using a visualization module 119, as illustrated in FIG. 3.

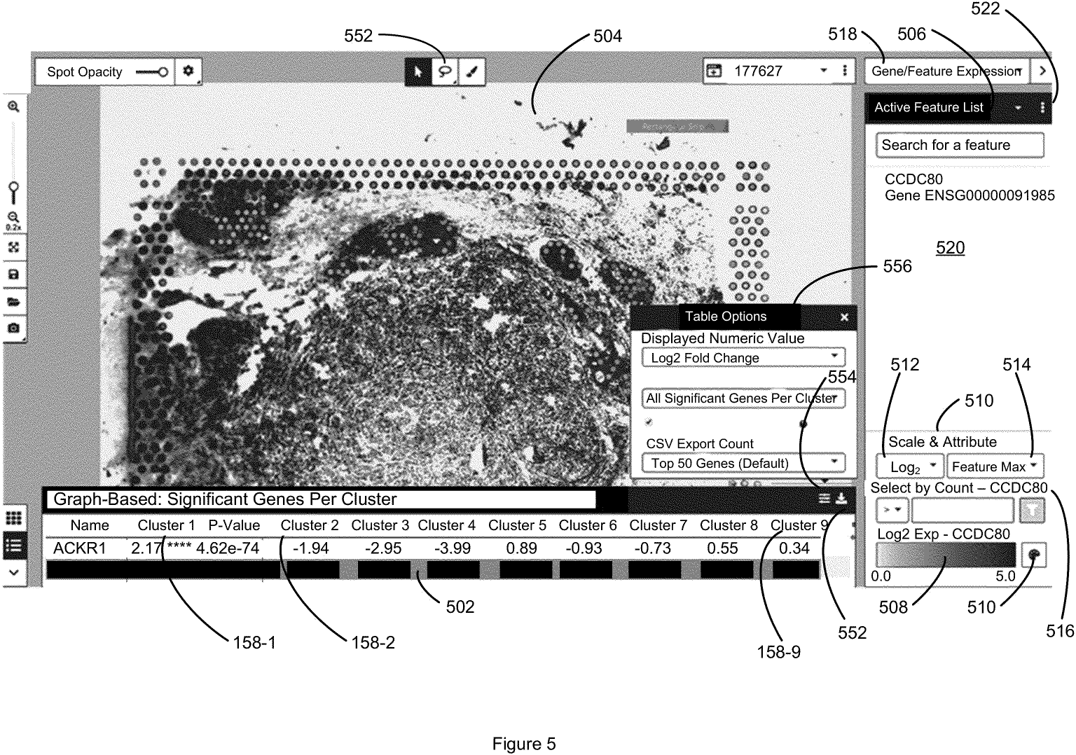

For instance, in some embodiments, each locus in a particular probe spot in the plurality of probe spots is barcoded with a respective barcode that is unique to the particular probe spot. FIG. 14 illustrates. In FIG. 14, a substrate 1402 containing marked capture areas (e.g., 6.5 × 6.5 mm) 1404 are used where tissue sections of a biological sample are placed and imaged to form images 125. Each capture area 1404 contains a number (e.g., 5000 printed regions) of barcoded mRNA capture probes, each such region referred to herein as probe spots 126 with dimensions of 100 µm or less (e.g., 55 µm in diameter and a center-to-center distance of 200 µm or less (e.g., 100 µm). Tissue is permeabilized and mRNAs are hybridized to the barcoded capture probes 1405 located proximally and/or directly underneath. As shown in more detail in panel 1406, for a particular capture probe 1405, cDNA synthesis connects the spatial barcode 1408 and the captured mRNA 1412, and sequencing reads, in the form of UMI counts, are later overlaid with the tissue image 125 as illustrated in FIG. 5. In FIG. 5, for each respective probe spot, the corresponding UMI counts, in log₂ space, mapping onto the gene CCDC80 are overlaid on the image 125. Returning to FIG. 14, for each respective probe spot 126, there are thousands or millions of capture probes 1405, with each respective capture probe 1405 containing the spatial barcode 1408 corresponding to the respective probe spot 126, and a unique UMI identifier 1410. The mRNA 1412 from the tissue sample binds to the capture probe 1405 and the mRNA sequence, along with the UMI 1410 and spatial barcode 1408 are copied in cDNA copies of the mRNA thereby ensuring that the spatial location of the mRNA within the tissue is captured at the level of probe spot 126 resolution.

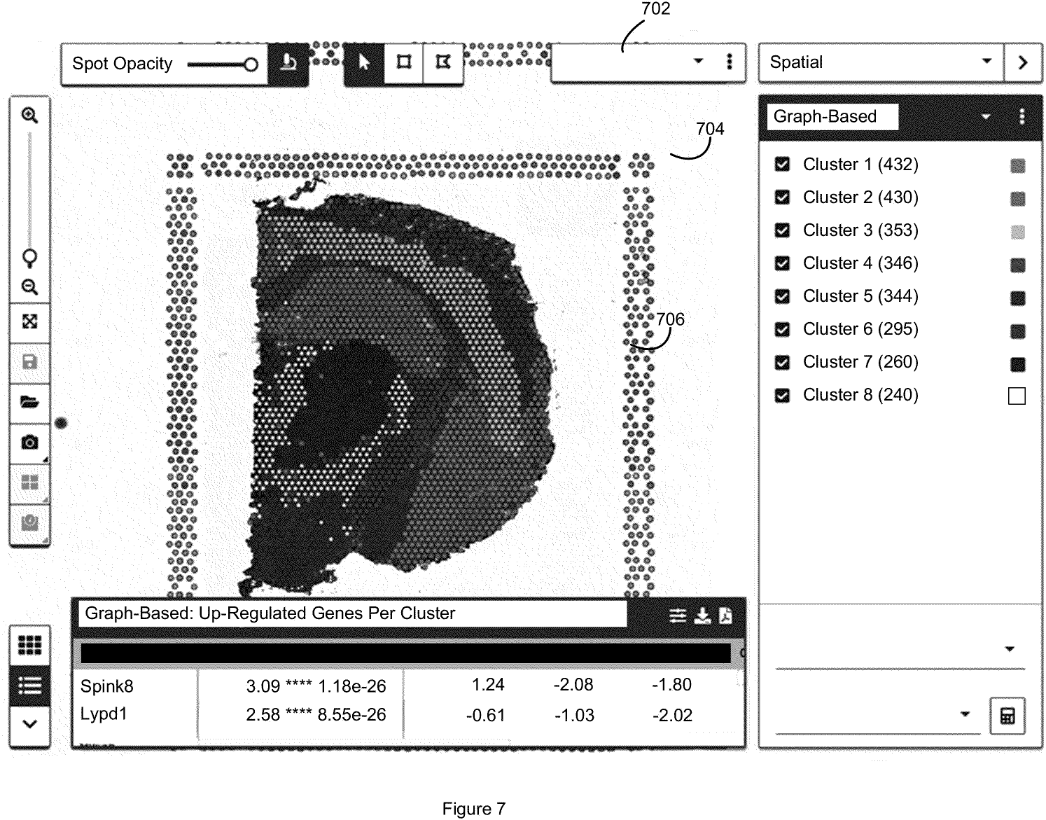

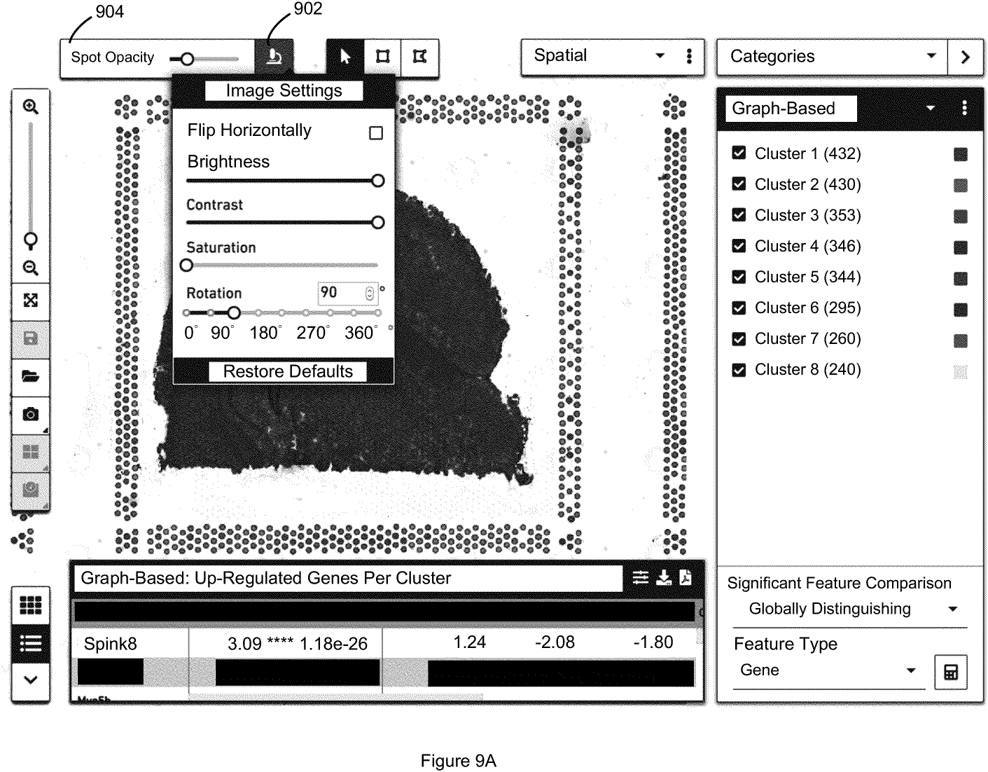

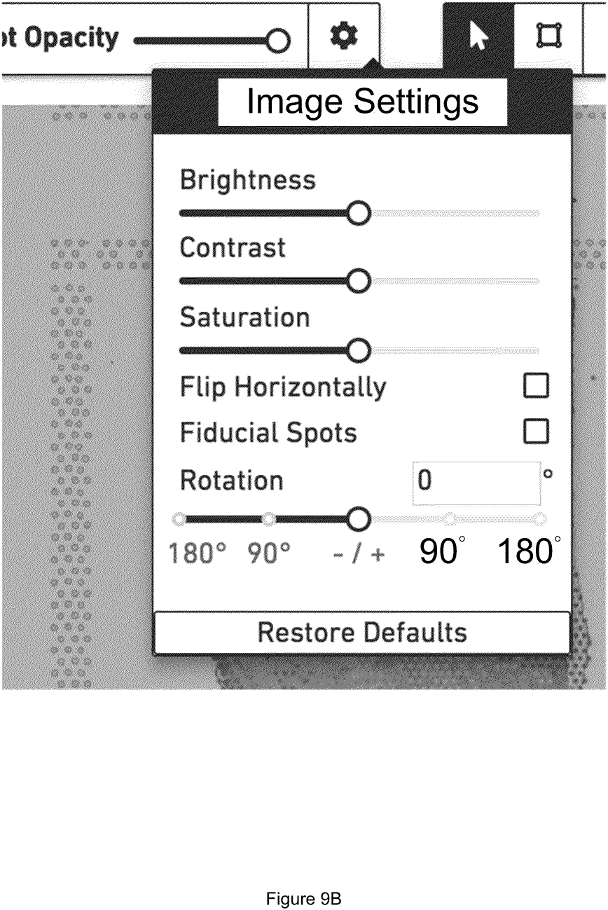

Moreover, in some embodiments, each capture area of an image 125 is indicated (e.g., outlined) by a plurality of printed fiduciary marks (e.g., to identify the location of each capture area). In some embodiments, each plurality of printed fiduciary dots (e.g., dots 706 in FIG. 7) is printed into a corresponding rectangle outlining each capture area. The fiduciary positions are stored in the discrete attribute value dataset 120 (e.g., a .cloupe file) as an additional projection, akin to the other spots in a .cloupe dataset. These fiduciary positions are viewable for spatial datasets by selecting “Fiduciary Spots” from the Image Settings panel, discussed herein, as shown in FIG. 9B. When selected, circles, or other closed-form geometric indicia such as rectangles stars, etc., that approximate the sizes of the fiduciary markers are superimposed on the image. Since the substrate creation process leaves visible spots in the locations of the fiduciary markers it follows that these fiduciary locations should ideally line up with the markers visible in the image. When they do, this provides confidence that the barcoded spots are in the correct position relative to the image. When they do not, they should prompt a user to attempt to realign the image. In some embodiments, fiduciary spots will appear as a single color of spots, or two colors of spots: the corner spots and remaining frame spots, atop the image. In some embodiments, fiduciary spots are toggleable in image settings.

Depending on the biological sample and the nature of analyte expression within the biological sample, morphological patterns obtained from spatial analysis of analytes can provide valuable insight into the underlying biological sample. For instance, the morphological patterns can be used to determine a disease state of the biological sample. As another example, the morphological pattern can be used to recommend a therapeutic treatment for the donor of the biological sample.

An example of the utility of the disclosed methods can be appreciated by considering the important biological question regarding whether lymphocytes have successfully infiltrated a tumor or not. Using the disclosed techniques, the lymphocytes may have different expression profiles then the tumor cells. Thus, the lymphocytes may cluster (e.g., through any of the clustering methods described herein) into a first cluster and thus each probe spot corresponding to portions of a tissue sample in which lymphocytes are present may have first indicia associated with the first cluster. The tumor cells may cluster into a second cluster and thus each probe spot in which lymphocytes are not present may have second indicia for the second cluster. When this is the case, the morphological pattern of lymphocyte infiltration into the tumor can be documented by probe spots bearing first indicia (representing the lymphocytes) amongst the probe spots bearing second indicia (representing the tumor cells). The morphological pattern exhibited by the lymphocyte infiltration into the tumor would be associated with a favorable diagnosis whereas the inability of lymphocytes to infiltrate the tumor would be associated with an unfavorable diagnosis. Thus, in this way, the spatial relationship (morphological pattern) of cell types in heterogeneous tissue can be used to analyze tissue samples.

Another example of the utility of the disclosed methods can be appreciated by considering the important biological question regarding whether a tumor is metastasizing or to determine the overall extent of a tumor within a normal healthy tissue (e.g., even in cases where the tumor is very small and difficult to discern by conventional visual methods). Using the disclosed techniques, the cancerous cells associated with the tumor will have different expression profiles than the normal cells. Thus, the cancerous cells may cluster (e.g., through any of the clustering methods described herein) into a first cluster using the disclosed methods and thus each probe spot corresponding to portions of a tissue sample in which the cancerous cells are present will have first indicia associated with the first cluster. The normal cells may cluster into a second cluster and thus each probe spot corresponding to portions of the tissue sample in which cancerous cells are not present will have second indicia for the second cluster. If this is the case, the morphological pattern of cancer cell metastasis, or the morphology of a tumor (e.g., shape and extent within a normal healthy tissue sample) can be documented by probe spots bearing first indicia (representing cancerous cells) amongst the probe spots bearing second indicia (representing normal cells).

Further details and non-limiting embodiments relating to methods for spatial analysis of analytes in biological samples, including removal of sample from the array, release and amplification of analytes, analysis of captured analytes (e.g., by sequencing and/or multiplexing), spatial resolution of analyte information (e.g., lookup tables, fiducial alignment and image alignment) are described in U.S. Pat. Publication No. US 2021-0062272 entitled “Systems and Methods for Using the Spatial Distribution of Haplotypes to Determine a Biological Condition,” filed Aug. 13, 2020; in U.S. Pat. Publication No. US 2021-0158522, entitled “SYSTEMS AND METHODS FOR SPATIAL ANALYSIS OF ANALYTES USING FIDUCIAL ALIGNMENT”; U.S. Pat. Publication No. US 2021-0150707, entitled “SYSTEMS AND METHODS FOR TISSUE CLASSIFICATION”; U.S. Pat. Publication No. US2021-0097684, entitled “Systems and Methods for Identifying Morphological Patterns in Tissue Samples”; and U.S. Pat. Publication No. US2021-0155982, entitled “Spatial Analysis of Analytes,” each of which is hereby incorporated herein by reference in its entirety.

### Exemplary System Embodiments

FIGS. 1A and 1B collectively illustrate a block diagram illustrating a visualization system 100 in accordance with some implementations. The device 100 in some implementations includes one or more processing units (CPU(s)) 102 (also referred to as processors), one or more network interfaces 104, a user interface 106 comprising a display 108 and an input module 110, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the visualization system 100 with other devices or a communication network;
a visualization module 119 for selecting a discrete attribute value dataset 120 and presenting information about the discrete attribute value dataset 120, where the discrete attribute value dataset 120 comprises a corresponding discrete attribute value 124 (e.g., count of transcript reads mapped to a single reference sequence) for each reference sequence 122 (e.g., single gene) in a plurality of reference sequences (e.g., a genome of a species) for each respective entity 126 (e.g., a nucleus and/or a probe spot) in a plurality of entities for each two-dimensional spatial arrangement 125 of the plurality of entities for each region of interest 121;
an optional clustering module 152 for clustering a discrete attribute value dataset 120 using the discrete attribute values 124 for each reference sequence 122 in the plurality of reference sequences for each respective entity 126 in the plurality of entities for each two-dimensional spatial arrangement 125 for each region of interest 121, or dimension reduction component values 164 derived therefrom, thereby assigning respective entities to clusters 158 in a plurality of clusters in a clustered dataset 128; and
optionally, all or a portion of a clustered dataset 128, the clustered dataset 128 comprising a plurality of clusters 158, each cluster 158 including a subset of entities 126, and each respective cluster 158 including a differential value 162 for each reference sequence 122 across the entities 126 of the subset of entities for the respective cluster 158.

In some implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

FIG. 1A illustrates that the clustered dataset 128 includes a plurality of clusters 158 comprising cluster 1 (158-1), cluster 2 (158-2) and other clusters up to cluster P (158-P), where P is a positive integer. Cluster 1 (158-1) is stored in association with entity 1 for cluster 1 (126-1-1), entity 2 for cluster 1 (126-2-1), and subsequent entities up to entity Q for cluster 1 (126-Q-1), where Q is a positive integer. As shown for cluster 1 (158-1), the cluster attribute value for entity 1 (160-1-1) is stored in association with the entity 1 for cluster 1 (126-1-1), the cluster attribute value for the entity 2 (160-2-1) is stored in association with the entity 2 for cluster 1 (126-2-1), and the cluster attribute value for the entity Q (160-Q-1) is stored in association with the entity Q for cluster 1 (126-Q-1). The clustered dataset 128 also includes differential value for reference sequence 1 for cluster 1 (162-1-1) and subsequent differential values up to differential value for reference sequence M for cluster 1 (162-1-M). Cluster 2 (158-2) and other clusters up to cluster P (158-P) in the clustered dataset 128 can include information similar to that in cluster 1 (158-1), and each cluster in the clustered dataset 128 is therefore not described in detail. A discrete attribute value dataset 120, which is store in the persistent memory 112, includes discrete attribute value dataset 120-1 and other discrete attribute value datasets up to discrete attribute value dataset 120-X.

Referring to FIG. 1B, persistent memory 112 stores one or more discrete attribute value datasets 120. Each discrete attribute value dataset 120 comprises one or more regions of interest 121. In some embodiments, a discrete attribute value dataset 120 comprises a single region of interest 121. In some embodiments, a discrete attribute value dataset 120 comprises a plurality of regions of interest. Each region of interest 121 has an independent set of spatial arrangements 125, and a distinct set of entity locations 123 comprising unique two-dimensional positions for the respective entities. However, in typical embodiments, a discrete attribute value dataset 120 contains a single feature barcode matrix. In other words, the entities used in each of the regions of interest 125 in a particular single given discrete attribute value dataset 120 are the same. Moreover, the entities used in each of the spatial arrangements of a particular region of interest 125 are the same. Accordingly, in some embodiments, each entity in a plurality of entities contains a suffix, or other form of indicator, that indicates which region of interest 121 a given entity (and subsequent measurements) originated. For instance, the barcode (e.g., for a respective capture probe) ATAAA-1 from region of interest (capture area) 1 (121-1-1) will be different from ATAAA-2 from region of interest (capture area) 2 (121-1-2).

As illustrated in FIG. 1B, in some embodiments, a spatial arrangement 125 comprises, for each respective entity 126 in a plurality of entities (associated with the corresponding dataset), a discrete attribute value 124 for each reference sequence 122 in a plurality of reference sequences. For example, as shown in FIG. 1B, a discrete attribute value dataset 120-1 (shown by way of example) includes information related to entity 1 (126-1-1-1), entity 2 (126-1-1-2) and other entities up to entity T (126-1-1-T) for each spatial arrangement 125 of each region of interest 121.

As shown for entity 1 (126-1-1-1) of spatial arrangement 125-1-1 of region of interest 121-1, the entity 1 (126-1-1-1) includes a discrete attribute value 124-1-1-1 of reference sequence 1 for entity 1 (122-1-1-1), a discrete attribute value 124-1-1-2 of reference sequence 2 for entity 1 (122-1-1-2), and other discrete attribute values up to discrete attribute value 124-1-1-M of reference sequence M for entity 1 (122-1-1-M). In some embodiments, each reference sequence is a different reference sequence in a reference genome. More generally, each reference sequence is a different feature (e.g., gene, locus, antibody, location in a reference genome, etc.).

In some embodiments, the dataset further stores a plurality of dimension reduction component values 164 and/or a two-dimensional data point and/or a category 170 assignment for each respective entity 126 in the plurality of entities. FIG. 1B illustrates, by way of example, dimension reduction component value 1 164-1-1 through dimension reduction component value N 164-1-N stored for entity 126-1, where N is positive integer.

FIG. 1B also illustrates how, in some embodiments, each entity is given a cluster assignment 158 (e.g., cluster assignment 158-1 for entity 1). In some embodiments, such clustering clusters based on discrete attribute values across all the spatial arrangements of all the regions of interest of a dataset. In some embodiment, some subset of the spatial arrangements, or some subset of the projections is used to perform the clustering.

FIG. 1B also illustrates one or more category assignments 170-1, ... 170-Q, where Q is a positive integer, for each entity (e.g., category assignment 170-1-1, ... 170-Q-1, for entity 1). In some embodiments, a category assignment includes multiple classes 172 (e.g., class 172-1, ..., 172-M, such as class 172-1-1, ..., 172-M-1 for entity 1, where M is a positive integer).

In some alternative embodiments, the discrete attribute value dataset 120 stores a two-dimensional data point 166 for each respective entity 126 in the plurality of entities (e.g., two-dimensional data point 166-1 for entity 1 in FIG. 1B) but does not store the plurality of dimension reduction component values 164.

In some embodiments, each entity represents a plurality of cells. In some embodiments, each entity represents a different individual cell (e.g., for liquid biopsy analysis where cells are disaggregated). In some embodiments, each entity represents a plurality of probe spots. In some embodiments, each entity represents a different individual probe spot (e.g., for spatial analysis where probe spots are arrayed on a substrate and/or for single cell analysis where probe spots are partitioned with individual cells). In some embodiments, each reference sequence represents a sequence of an analyte measured in each different entity. In some embodiments, each reference sequence represents an mRNA measured in a respective entity that maps to a respective gene in the genome of the cell, and the dataset further comprises the total RNA counts per entity. In some embodiments, referring to FIGS. 1A and 1B, each discrete attribute value 124 for each respective entity is a discrete attribute value for each reference sequence in a plurality of reference sequences for the respective entity in the plurality of entities.

Although FIGS. 1A and 1B depict a “visualization system 100,” the figures are intended more as functional description of the various features that may be present in computer systems than as a structural schematic of the implementations described herein. In practice, and as recognized by those of ordinary skill in the art, items shown separately could be combined and some items could be separated. Moreover, although FIGS. 1A and 1B depict certain data and modules in non-persistent memory 111, some or all of these data and modules may be in persistent memory 112. Further, while discrete attribute value dataset 120 is depicted as resident in persistent memory 112, a portion of discrete attribute value dataset 120 is, in fact, resident in non-persistent memory 111 at various stages of the disclosed methods.

### Example Embodiments

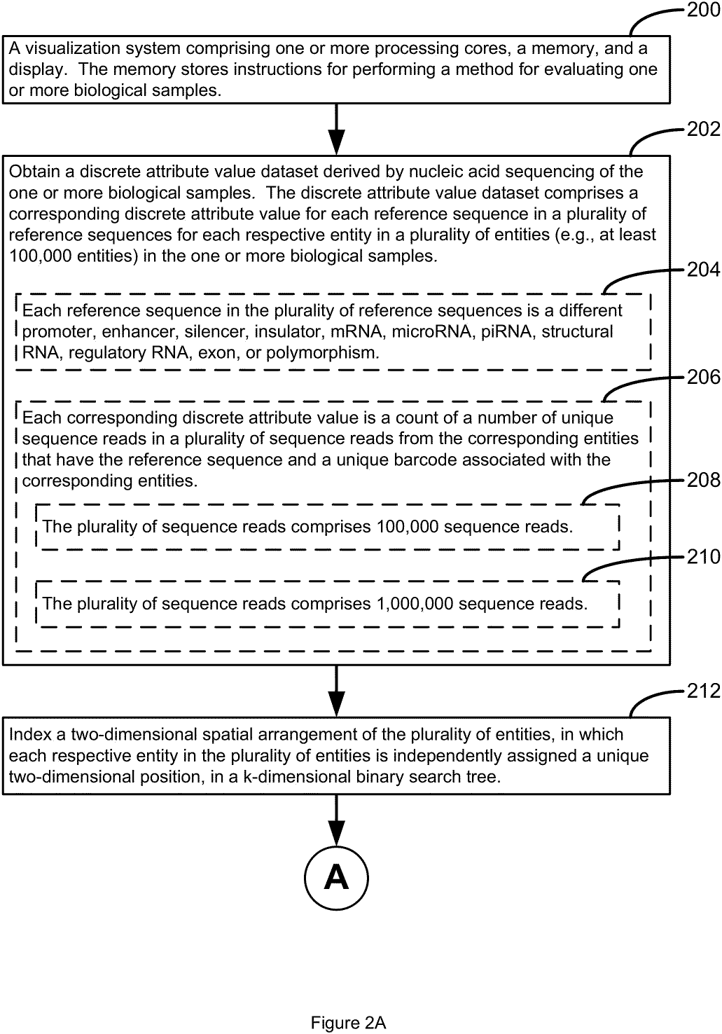

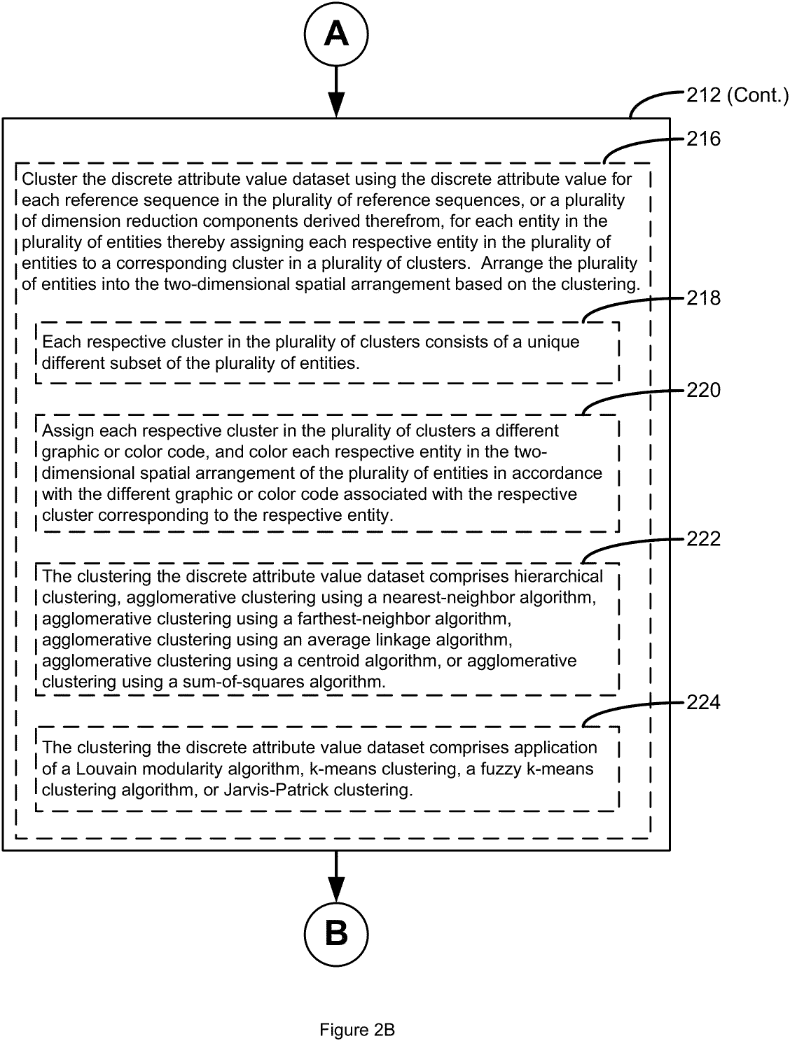

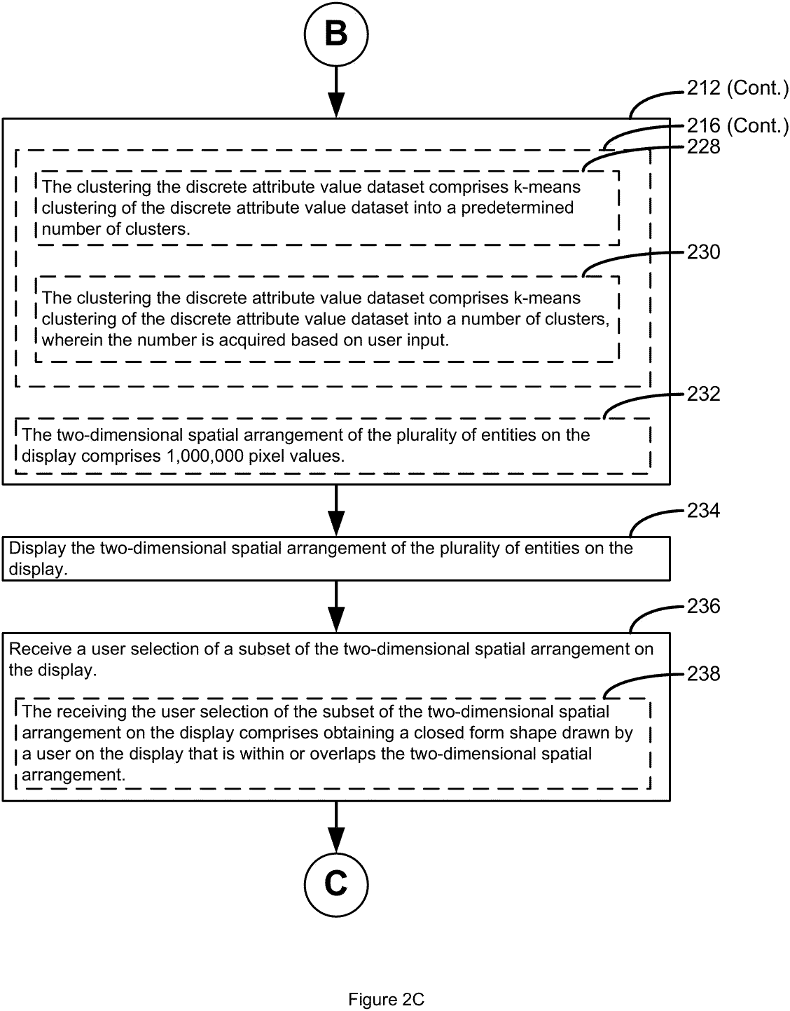

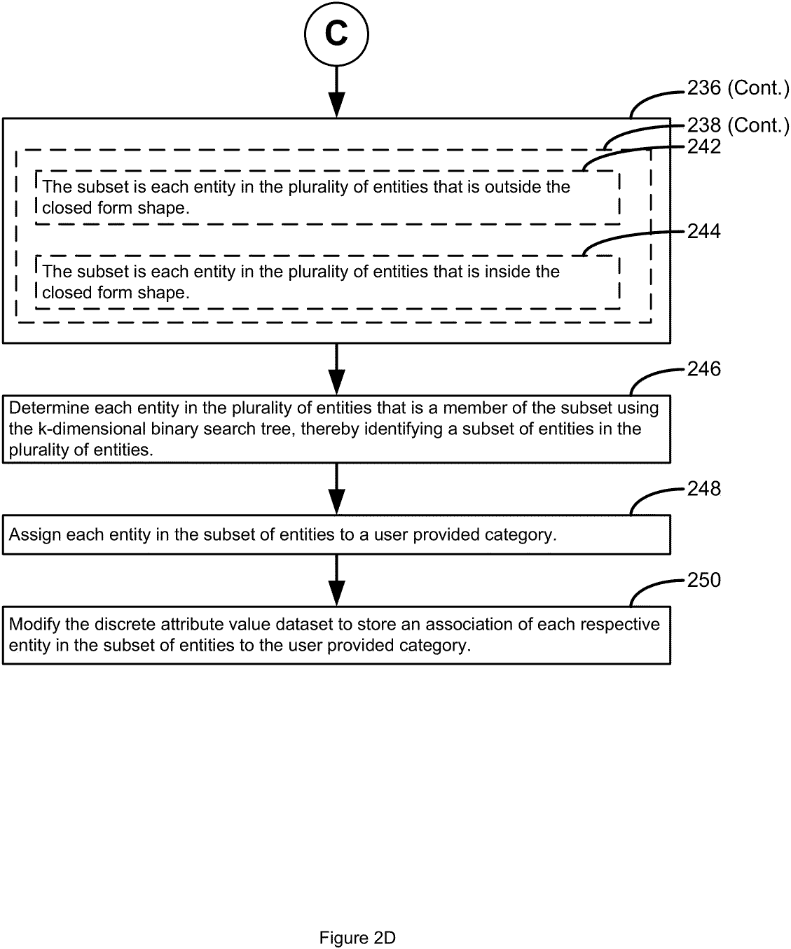

While a system in accordance with the present disclosure has been disclosed with reference to FIGS. 1A-C, visualization systems for performing a method in accordance with the present disclosure are now detailed with reference to FIGS. 2A-D and 30A-B.

Referring to Block 200 of FIGS. 2A-D, one aspect of the present disclosure provides a visualization system comprising one or more processing cores, a memory, and a display, the memory storing instructions for performing a method for evaluating one or more biological samples.

### Discrete Attribute Value Datasets

Referring to Block 202, the method comprises obtaining a discrete attribute value dataset derived by nucleic acid sequencing (e.g., single cell or single nuclei sequencing) of the one or more biological samples, where the discrete attribute value dataset comprises a corresponding discrete attribute value for each reference sequence in a plurality of reference sequences for each respective entity in a plurality of entities (e.g., at least 100,000 entities) in the one or more biological samples.

In some embodiments, the one or more biological samples comprises at least 2, at least 3, at least 4, at least 5, at least 6, at least 7, at least 8, at least 9, at least 10, at least 15, at least 20, at least 30, at least 40, at least 50, or at least 100 biological samples. In some embodiments, the one or more biological samples comprises no more than 300, no more than 100, no more than 50, no more than 30, no more than 20, no more than 10, or no more than 5 biological samples. In some embodiments, the one or more biological samples is from 2 to 10, from 5 to 20, from 3 to 50, or from 20 to 100 biological samples.

In some embodiments, an entity is a cell. In some embodiments, an entity is a nucleus (e.g., a cell nucleus). In some embodiments, each respective entity in the plurality of entities corresponds to a respective cell in the one or more biological samples. Accordingly, in some embodiments, each respective entity in the plurality of entities is a nucleus of a cell in the one or more biological samples. In some embodiments, a respective entity in the plurality of entities is a visual representation of a physical nucleus, where the visual representation of the respective nucleus is provided in a two-dimensional spatial arrangement (e.g., an image or a representation thereof) of the plurality of entities.

In some embodiments, an entity is a probe spot. In some embodiments, each respective entity in the plurality of entities corresponds to a respective probe spot in a plurality of probe spots. Accordingly, in some embodiments, each respective entity in the plurality of entities is a respective probe spot in a plurality of probe spots. In some embodiments, a respective entity in the plurality of entities is a visual representation of a physical probe spot, where the visual representation of the respective probe spot is provided in a two-dimensional spatial arrangement (e.g., an image or a representation thereof) of the plurality of probe spots.

In some embodiments, the plurality of entities comprises at least 1000, at least 2000, at least 3000, at least 4000, at least 5000, at least 10,000, at least 20,000, at least 50,000, at least 100,000, at least 200,000, at least 300,000, at least 400,000, at least 500,000, at least 600,000, at least 700,000, at least 800,000, at least 900,000, at least 1 million, at least 2 million, at least 3 million, at least 5 million, or at least 10 million entities. In some embodiments, the plurality of entities comprises no more than 50 million, no more than 10 million, no more than 5 million, no more than 1 million, no more than 500,000, no more than 100,000, no more than 50,000, no more than 10,000, or no more than 5000 entities. In some embodiments, the plurality of entities comprises from 5000 to 100,000, from 50,000 to 500,000, from 100,000 to 2 million, or from 500,000 to 10 million entities. In some embodiments, the plurality of entities falls within another range starting no lower than 1000 entities and ending no higher than 50 million entities.

In some embodiments, the discrete attribute value dataset comprises abundance data for one or more analytes. For instance, in some embodiments, the corresponding discrete attribute value for each reference sequence in the plurality of reference sequences is an abundance of a nucleic acid sequence that maps to the respective reference sequence. In some embodiments, the plurality of reference sequences comprises at least 3, at least 4, at least 5, at least 10, at least 15, at least 20, at least 25, at least 30, at least 40, at least 50, at least 60, at least 70, at least 80, at least 90, at least 100, at least 200, at least 300, at least 500, at least 1000, at least 2000, or at least 5000 reference sequences. In some embodiments, the plurality of reference sequences comprises no more than 10,000, no more than 5000, no more than 2000, no more than 1000, no more than 500, no more than 100, no more than 50, or no more than 20 reference sequences. In some embodiments, the plurality of reference sequences comprises from 3 to 50, from 10 to 200, from 100 to 1000, or from 500 to 10,000 reference sequences. In some embodiments, the plurality of reference sequences falls within another range starting no lower than 3 reference sequences and ending no higher than 10,000 reference sequences.

Referring to Block 204, in some embodiments, each reference sequence in the plurality of reference sequences is a different promoter, enhancer, silencer, insulator, mRNA, microRNA, piRNA, structural RNA, regulatory RNA, exon, or polymorphism.

In some embodiments, each reference sequence in the plurality of reference sequences is a respective gene. In some embodiments, each reference sequence in the plurality of reference sequences is a respective locus.

In some embodiments, the discrete attribute value dataset 120 is obtained using a nucleic acid sequencing. In some embodiments, the discrete attribute value dataset 120 represents a transcriptome sequencing that quantifies gene expression from an entity (e.g., a nucleus and/or a probe spot) in counts of transcript reads mapped to the genes. In some embodiments, the discrete attribute value dataset 120 is obtained using a whole transcriptome sequencing (e.g., RNA-seq). In some embodiments, a discrete attribute value dataset 120 is obtained using a sequencing experiment in which baits are used to selectively filter and pull down a gene set of interest as disclosed, for example, in U.S. Patent Application No. 17/239,555, entitled “Capturing Targeted Genetic Targets Using a Hybridization/Capture Approach,” filed Apr. 24, 2021, which is hereby incorporated by reference. In some embodiments, the discrete attribute value dataset represents a whole transcriptome shotgun sequencing experiment that quantifies gene expression from a single entity (e.g., a nucleus and/or a probe spot) in counts of transcript reads mapped to genes.

In some embodiments, discrete attribute value dataset 120 is obtained using droplet based single-cell RNA-sequencing (scRNA-seq). For instance, a droplet based single-cell RNA-sequencing microfluidics system can be used to enable 3' or 5' messenger RNA (mRNA) digital counting of thousands of individual entities 126 (e.g., single cells). In some such embodiments, sequencing by a droplet-based platform is used to perform barcoding of cells.

In some embodiments, discrete attribute value dataset 120 is obtained using RNA templated ligation (e.g., spatial RNA templated ligation) as described in, for instance, U.S. Pat. Application Nos. US 2021-0348221 and US 2021-0285046.

Various sequencing schemes can be employed. For example, in some embodiments, the sequencing is sequencing by synthesis, sequencing by hybridization, sequencing by ligation, nanopore sequencing, sequencing using nucleic acid nanoballs, pyrosequencing, single molecule sequencing (e.g., single molecule real time sequencing), single cell/entity sequencing, massively parallel signature sequencing, polony sequencing, combinatorial probe anchor synthesis, SOLiD sequencing, chain termination (e.g., Sanger sequencing), ion semiconductor sequencing, tunneling currents sequencing, heliscope single molecule sequencing, sequencing with mass spectrometry, transmission electron microscopy sequencing, RNA polymerase-based sequencing, or any other method, or a combination thereof. In some embodiments, the sequencing is a sequencing technology like Heliscope (Helicos), SMRT technology (Pacific Biosciences) or nanopore sequencing (Oxford Nanopore) that allows direct sequencing of single molecules without prior clonal amplification. In some embodiments, the sequencing is performed with or without target enrichment. In some embodiments, the sequencing is Helicos True Single Molecule Sequencing (tSMS) (e.g., as described in Harris T. D. et al., Science 320:106-109 [2008]). In some embodiments, the sequencing is 454 sequencing (Roche) (e.g., as described in Margulies, M. etal. Nature 437:376-380 (2005)). In some embodiments, the sequencing is SOLiD™ technology (Applied Biosystems). In some embodiments, the sequencing is single molecule, real-time (SMRT™) sequencing technology of Pacific Biosciences. In some embodiments, the systems and methods described herein are used with any sequencing platform, including, but not limited to, Illumina NGS platforms, Ion Torrent (Thermo) platforms, and GeneReader (Qiagen) platforms.

In some embodiments, the discrete attribute value dataset 120 is obtained from a single nucleus-based nucleic acid sequencing, such as single nuclei RNA sequencing (snRNA-seq). For instance, as described above, snRNA-seq can be used to measure RNA expression from isolated nuclei as opposed to RNA of an entire cell (e.g., cytoplasmic RNA plus nuclear RNA). In some embodiments, the discrete attribute value dataset 120 is obtained from single cell nucleic acid sequencing. Single cell nucleic acid sequencing can include, for instance, single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, and any combination thereof. The sequencing technique can be selected based on the desired analyte to be measured. For instance, scRNA-seq, scTag-seq, and miRNA-seq can be used to measure RNA expression. Specifically, scRNA-seq measures expression of RNA transcripts, scTag-seq allows detection of rare mRNA species, and miRNA-seq measures expression of micro-RNAs. CyTOF/SCoP and E-MS/Abseq can be used to measure protein expression in the cell. See, Definitions: Entity, above.

Thus, referring to Block 206, in some embodiments, each corresponding discrete attribute value is a count of a number of unique sequence reads in a plurality of sequence reads from the corresponding entities that have the reference sequence and a unique barcode associated with the corresponding entities. For instance, in some embodiments, each corresponding discrete attribute value is an abundance (e.g., an mRNA abundance) for each corresponding entity that has the reference sequence (e.g., the respective analyte) and a unique barcode associated with the corresponding entity. In some embodiments, the abundance is an absolute abundance, a relative abundance, a fold change, or a log-transformed abundance.

In some embodiments, the discrete attribute value dataset is obtained using an RNA sequencing reaction for bulk RNAseq (standard RNAseq). In some embodiments, the discrete attribute value dataset is obtained using an RNA sequencing reaction for single cell RNAseq.

In some embodiments, the plurality of sequence reads are obtained by single cell 3’ sequencing, single cell 5’ sequencing, or single cell 5’ paired-end sequencing. See, for example, Voet et al., 2013, “Single-cell paired-end genome sequencing reveals structural variation per cell cycle,” Nucleic Acids Res 41: 6119-6138, Zong et al., 2012, “Genome-wide detection of single nucleotide and copy-number variations of a single human cell,” Science 338, pp. 1622-1626; Navin et al., 2011, Tumour evolution inferred by single-cell sequencing,” Nature 472, pp. 90-94, Snyder et al., 2012, “Clonal Evolution of Preleukemic Hematopoietic Stem Cells Precedes Human Acute Myeloid Leukemia,” Science Translational Medicine 4, 149ra118, and Bourcy et al., 2014, “A Quantitative Comparison of Single-Cell Whole Genome Amplification Methods,” PLOS ONE 9(8), e105585, each of which is hereby incorporated by reference.

In some embodiments, the single cell 3’sequencing, single cell 5’ sequencing, or single cell 5’ paired-end sequencing is performed by preparing 3' gene expression libraries and/or 5’ gene expression libraries. In some embodiments, the 3' and/or 5' gene expression libraries are prepared using oligo-dT primers to amplify the 3' ends of nucleic acid sequences. In some such embodiments, 3' and 5' gene expression libraries are prepared using different methods.

In some exemplary embodiments, 3' gene expression libraries are prepared from RNA using a reverse transcription step in which the poly-A tail at the 3' end of the RNA sequence is hybridized to a capture probe attached to a capture bead. The capture probe contains an oligo-dT sequence at the free end. Reverse transcription provides a first-strand cDNA synthesis that occurs directly on the capture probe in the 3' to 5' direction of the template RNA strand, creating a template or antisense strand of cDNA extending from the capture probe attached to the capture bead. The cDNA template strand further comprises an untemplated C-C-C... nucleotide sequence on the free end of the newly synthesized cDNA fragment, as a byproduct of the reverse transcriptase. The original RNA sequence then dissociates from the capture probe, leaving the newly extended capture probe free for hybridization and amplification. A template switch oligonucleotide hybridizes to the untemplated C-C-C... sequence, thus priming the cDNA sequence for amplification in the opposite direction. In some embodiments, the capture probe comprises an optional sequence that is complimentary to a primer sequence for hybridization and amplification. In some such embodiments, the extended capture probe is subsequently amplified using the template switch oligonucleotide and/or the primer sequence complimentary to a sequence on the capture probe. In some other such embodiments, the capture probe comprises additional sequences, including a barcode, a spatial barcode, a UMI, or a functional sequence such as a sequencing adaptor.

In some other exemplary embodiments, 5’ gene expression libraries are prepared from RNA using a reverse transcription step in which the poly-A tail at the 3' end of the RNA sequence is hybridized to a free oligo-dT primer that is not attached to a capture probe. The oligo-dT primer facilitates first-strand cDNA synthesis from the 3' to 5' direction of the original RNA strand, creating a template or antisense strand of cDNA. The newly synthesized cDNA template strand further comprises an untemplated C-C-C... nucleotide sequence on the 3' end, as a byproduct of the reverse transcriptase. The untemplated C-C-C... sequence of the newly synthesized cDNA fragment then hybridizes to a capture probe comprising a template switch oligonucleotide sequence. In some embodiments, the capture probe is attached to a capture bead and the template switch oligonucleotide sequence is located at the free end of the capture probe. The capture probe is extended along the length of the hybridized cDNA sequence, providing for a second strand cDNA amplification step. The original cDNA strand dissociates from the capture probe, leaving the newly extended capture probe available for further hybridization and amplification. In some embodiments, the capture probe comprises an optional sequence that is complimentary to a primer sequence for hybridization and amplification. In some such embodiments, the extended capture probe is subsequently amplified using the template switch oligonucleotide and/or the primer sequence complimentary to a sequence on the capture probe. In some other such embodiments, the capture probe comprises additional sequences, including a barcode, a spatial barcode, a UMI, or a functional sequence such as a sequencing adaptor. (See, 10X Genomics, “What is a template switch oligo (TSO)?,” available on the Internet at kb.10xgenomics.com/hc/en-us/articles/360001493051-What-is-a-template-switch-oligo-TSO-, the entire contents of which are incorporated herein by reference).

In some embodiments, paired end sequencing is performed in order to sequence both ends of a nucleic acid sequence fragment and generate high-quality, mappable sequence data. In some embodiments, a respective capture probe comprises a sequencing adaptor that is appended to the 5’ end of a sequence read during the preparation of 5' gene expression libraries. In some such embodiments, the sequencing adaptor facilitates sequencing from the 5’ end of the sequence read fragment. In some such embodiments, sequencing from the 3’end of the sequence read fragment is performed using primers complementary to the poly-A tail of the sequence read fragment. In some other such embodiments, sequencing from the 3’end of the sequence read fragment is performed using adaptors at the 3' end of the sequence read.

Additional embodiments for single cell sequencing are further disclosed herein (see, e.g., Definitions: (B) Methods for Single Cell Analysis of Analytes, above) and in, e.g., PCT Publication No. WO 2022/020728, entitled “Systems and Methods for Detecting and Removing Aggregates for Calling Cell-Associated Barcodes”; U.S. Pat. Publication No. US 2021-0332354 A1, entitled “Systems and Methods for Identifying Differential Accessibility of Gene Regulatory Elements at Single Cell Resolution”; U.S. Pat. Publication No. US 2021-0381056, entitled “SYSTEMS AND METHODS FOR JOINT INTERACTIVE VISUALIZATION OF GENE EXPRESSION AND DNA CHROMATIN ACCESSIBILITY”; U.S. Pat. Publication No. US 2019-0332963, entitled, “Systems and Methods for Visualizing a Pattern in a Dataset,”; and U.S. Pat. Application No. 17/239,555, entitled “Capturing Targeted Genetic Targets Using a Hybridization/Capture Approach,” filed Apr. 24, 2021, each of which is hereby incorporated herein by reference in its entirety.

Referring to Block 208, in some embodiments, the plurality of sequence reads comprises 100,000 sequence reads. Referring to Block 210, in some embodiments, the plurality of sequence reads comprises 1,000,000 sequence reads. In some embodiments, the plurality of sequence reads comprises at least 10,000, at least 20,000, at least 50,000, at least 100,000, at least 200,000, at least 300,000, at least 400,000, at least 500,000, at least 600,000, at least 700,000, at least 800,000, at least 900,000, at least 1 million, at least 2 million, at least 3 million, at least 5 million, at least 10 million, at least 50 million, or at least 100 million sequence reads. In some embodiments, the plurality of sequence reads comprises no more than 200 million, no more than 50 million, no more than 10 million, no more than 5 million, no more than 1 million, no more than 500,000, no more than 100,000, no more than 50,000, or no more than 10,000 sequence reads. In some embodiments, the plurality of sequence reads comprises from 10,000 to 100,000, from 50,000 to 500,000, from 100,000 to 2 million, or from 500,000 to 10 million sequence reads. In some embodiments, the plurality of sequence reads falls within another range starting no lower than 10,000 sequence reads and ending no higher than 200 million sequence reads.

Accordingly, in some embodiments, the discrete attribute value dataset comprises at least 10,000, at least 20,000, at least 50,000, at least 100,000, at least 200,000, at least 300,000, at least 400,000, at least 500,000, at least 600,000, at least 700,000, at least 800,000, at least 900,000, at least 1 million, at least 2 million, at least 3 million, at least 5 million, at least 10 million, at least 50 million, or at least 100 million discrete attribute values. In some embodiments, the discrete attribute value dataset comprises no more than 200 million, no more than 50 million, no more than 10 million, no more than 5 million, no more than 1 million, no more than 500,000, no more than 100,000, no more than 50,000, or no more than 10,000 discrete attribute values. In some embodiments, the discrete attribute value dataset comprises from 10,000 to 100,000, from 50,000 to 500,000, from 100,000 to 2 million, or from 500,000 to 10 million discrete attribute values. In some embodiments, the discrete attribute value dataset falls within another range starting no lower than 10,000 discrete attribute values and ending no higher than 200 million discrete attribute values.

In some embodiments, each nucleus in a plurality of nuclei corresponds to one or more respective probe spots in a plurality of probe spots. In some embodiments, each respective probe spot in a plurality of probe spots corresponds to one or more respective nuclei in a plurality of nuclei (see, e.g., Definitions: Entity, above).

In some embodiments, each respective nucleus in a plurality of nuclei corresponds to a respective probe spot in a corresponding plurality of probe spots. In some embodiments, each respective probe spot in a plurality of probe spots corresponds to a respective nucleus in a plurality of nuclei. Thus, in some embodiments, any methods and/or embodiments comprising the analysis, arrangement, and/or visualization of the plurality of nuclei for the one or more biological samples disclosed herein can be similarly applied to a plurality of probe spots associated with discrete attribute values for the one or more biological samples. Similarly, in some embodiments, any methods and/or embodiments comprising the analysis, arrangement, and/or visualization of the plurality of probe spots for the one or more biological samples disclosed herein can be similarly applied to a plurality of nuclei for the one or more biological samples.

In some embodiments, the discrete attribute value dataset 120 includes discrete attribute values 124 for the analytes of 50 or more probe spots, 100 or more probe spots, 250 or more probe spots, 500 or more probe spots, 5000 or more probe spots, 100,000 or more probe spots, 250,000 or more probe spots, 500,000 or more probe spots, 1,000,000 or more probe spots, 10 million or more probe spots, or 50 million or more probe spots. In some embodiments, the discrete attribute value dataset 120 includes discrete attribute values for 50 or more, 100 or more, 250 or more, 500 or more, 1000 or more, 3000 or more, 5000 or more, 10,000 or more, or 15,000 or more analytes in each probe spot 126 represented by the dataset.

In some embodiments, the discrete attribute value dataset 120 includes discrete attribute values for 25 or more, 50 or more, 100 or more, 250 or more, 1000 or more, 3000 or more, 5000 or more, 10,000 or more, or 15,000 or more loci 122 in each probe spot 126 represented by the dataset. In some such embodiments, the discrete attribute value dataset 120 includes discrete attribute values 124 for the loci of 500 or more probe spots, 5000 or more probe spots, 100,000 or more probe spots, 250,000 or more probe spots, 500,000 or more probe spots, 1,000,000 or more probe spots, 10 million or more probe spots, or 50 million or more probe spots in the discrete attribute value dataset 120.

In some embodiments, nucleic acids (e.g., mRNA) for more than 50, more than 100, more than 500, or more 1000 different genetic loci are localized to a single probe spot, and for each such respective genetic loci, one or more UMI are identified, meaning that there were one or more nucleic acid (e.g., mRNA) genetic loci encoding the respective genetic loci. In some embodiments, more than ten, more than one hundred, more than one thousand, or more than ten thousand UMI for a respective genetic locus are localized to a single probe spot. In some embodiments, the discrete attribute value dataset 120 includes discrete attribute values for the mRNAs of 500 or more probe spots, 5000 or more probe spots, 100,000 or more probe spots, 250,000 or more probe spots, 500,000 or more probe spots, 1,000,000 or more probe spots, 10 million or more probe spots, or 50 million or more probe spots within the discrete attribute value dataset 120. In some such embodiments, each such discrete attribute value is the count of the number of unique UMI that map to a corresponding genetic locus within a corresponding probe spot.

Accordingly, in some such embodiments, the discrete attribute value dataset 120 includes discrete attribute values for 5 or more, 10 or more, 25 or more, 35 or more, 50 or more, 100 or more, 250 or more 500 or more, 1000 or more, 3000 or more, 5000 or more, 10,000 or more, or 15,000 or more different mRNAs, in each probe spot represented by the dataset. In some embodiments, each such mRNA represents a different gene and thus the discrete attribute value dataset 120 includes discrete attribute values for 5 or more, 10 or more, 25 or more, 35 or more, 50 or more, 100 or more, 250 or more, 500 or more, 1000 or more, 3000 or more, 5000 or more, 10,000 or more, or 15,000 or more different genes in each probe spot represented by the dataset. In some embodiments, each such mRNA represents a different gene and the discrete attribute value dataset 120 includes discrete attribute values for between 5 and 20,000 different genes, or variants of different genes or open reading frames of different genes, in each probe spot represented by the dataset. More generally, in some such embodiments, the discrete attribute value dataset 120 includes discrete attribute values for 5 or more, 10 or more, 25 or more, 35 or more, 50 or more, 100 or more, 250 or more 500 or more, 1000 or more, 3000 or more, 5000 or more, 10,000 or more, or 15,000 or more different analytes, in each probe spot represented by the dataset, where each such analyte is a different gene, protein, cell surface feature, mRNA, intracellular protein, metabolite, V(D)J sequence, immune cell receptor, or perturbation agent. For general disclosure on how such analytes are spatially quantified, see, U.S. Pat. Application No. 16/951,864, entitled “Pipeline for Analysis of Analytes,” filed Nov. 18, 2020, which is hereby incorporated by reference. For general disclosure on how ATAC is spatially quantified using, for example clustering and/or t-SNE (where such cluster and/or t-SNE plots can be displayed in linked windows), see, U.S. Publication No. US-2020105373-A1 entitled “Systems and Methods for Cellular Analysis Using Nucleic Acid Sequencing” which is hereby incorporated by reference. For general disclosure on how V(D)J sequences are spatially quantified using, for example clustering and/or t-SNE (where such cluster and/or t-SNE plots can be displayed in linked windows), see, U.S. Pat. Publication No. US 2018-0371545, entitled “Systems and Methods for Clonotype Screening,” filed May 19, 2018, which is hereby incorporated by reference.

In some embodiments, a discrete attribute value dataset 120 has a file size of more than 1 megabytes, more than 5 megabytes, more than 100 megabytes, more than 500 megabytes, or more than 1000 megabytes. In some embodiments, a discrete attribute value dataset 120 has a file size of between 0.5 gigabytes and 25 gigabytes. In some embodiments, a discrete attribute value dataset 120 has a file size of between 0.5 gigabytes and 100 gigabytes.

### Spatial Arrangements

Referring to Block 212, the method further includes indexing a two-dimensional spatial arrangement of the plurality of entities, in which each respective entity in the plurality of entities is independently assigned a unique two-dimensional position, in a k-dimensional binary search tree.

In some embodiments, the two-dimensional spatial arrangement of the plurality of entities comprises an image of the one or more biological samples (see, e.g., Definitions: Imaging and Images, above). In some embodiments the two-dimensional spatial arrangement of the plurality of entities is obtained by aligning a plurality of analyte data with an image of the one or more biological samples, using any of the methods disclosed herein (see, e.g., Definitions: Spatial Analyte Data and Definitions: (C) Methods for Spatial Analysis of Analytes, above). In some embodiments, the two-dimensional spatial arrangement of the plurality of entities comprises an overlay of analyte data on an image of the one or more biological samples.

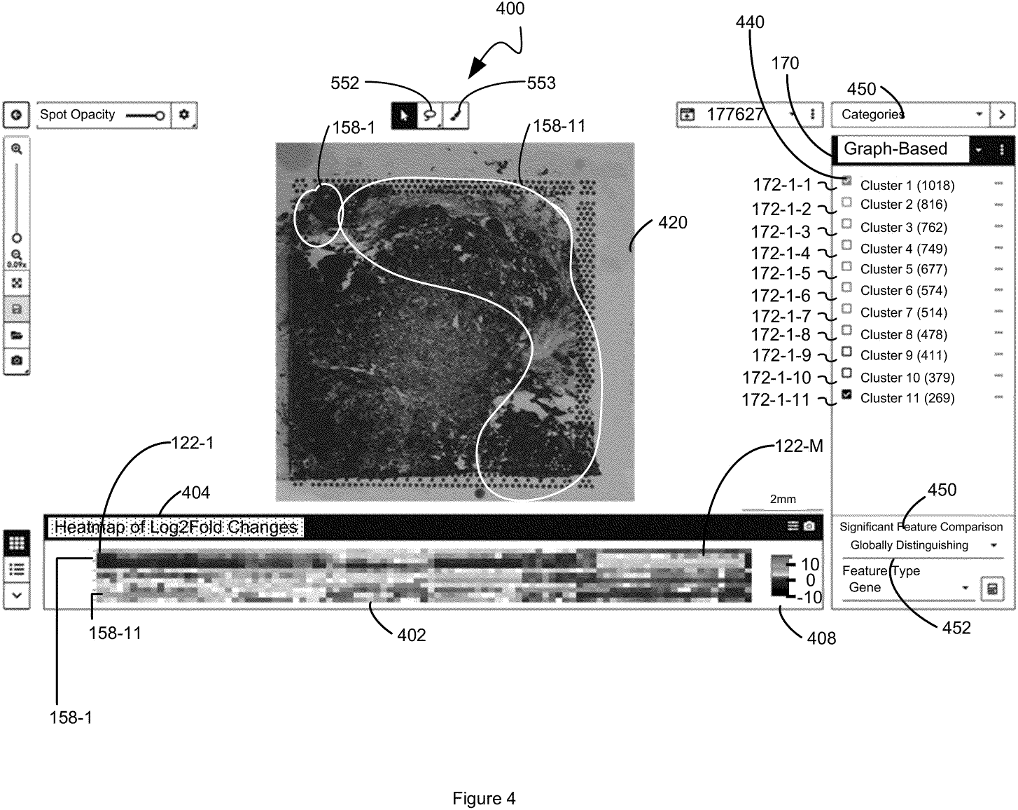

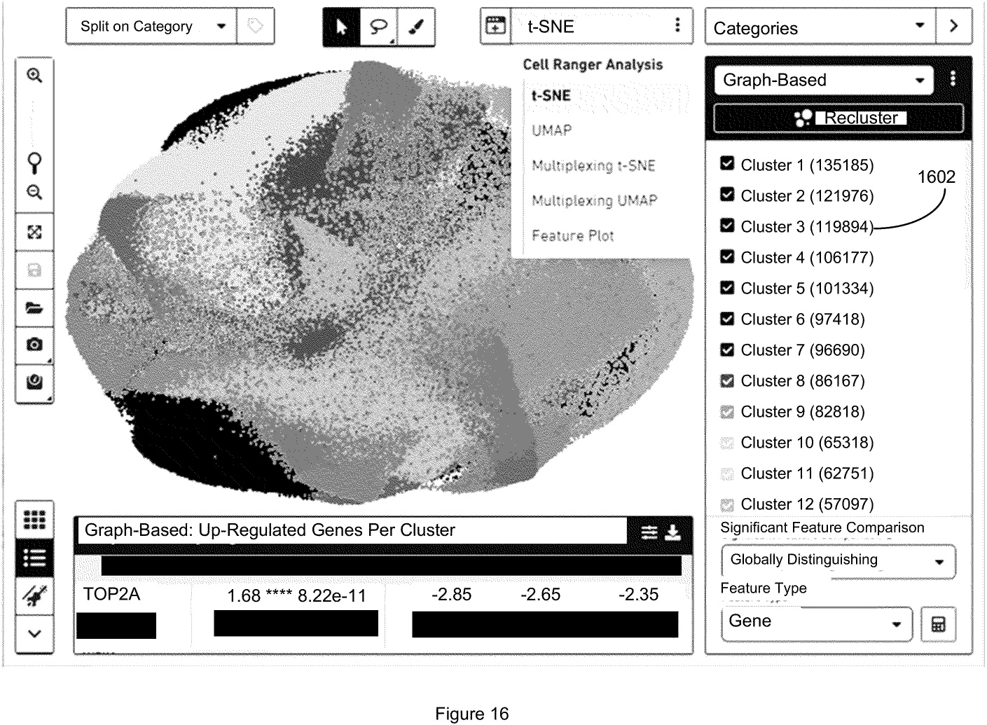

In some embodiments, the two-dimensional spatial arrangement is obtained using a graphical representation of an analysis of analyte data. For instance, in some embodiments, the two-dimensional spatial arrangement is obtained using clustering of analyte data. For instance, in some embodiments, the clustering is performed using the clustering module 152 of the visualization module 119 with the discrete attribute value dataset 120. FIGS. 4 and 16 illustrate visualizations of such clustering as performed using a user interface (as shown in FIG. 3). In FIG. 4, the clustering results are displayed on top of the underlying spatial arrangement 125 in panel 420. In FIG. 16, clustering is illustrated as a t-SNE plot, where each respective cluster 1602 is represented by applying a different color indicium to each respective entity in the plurality of entities that belongs to the respective cluster.

Accordingly, referring to Block 216, in some embodiments, the method further comprises clustering the discrete attribute value dataset using the discrete attribute value for each reference sequence in the plurality of reference sequences, or a plurality of dimension reduction components derived therefrom, for each entity in the plurality of entities thereby assigning each respective entity in the plurality of entities to a corresponding cluster in a plurality of clusters, and arranging the plurality of entities into the two-dimensional spatial arrangement based on the clustering.

In some embodiments, each respective cluster in the plurality of clusters contains overlapping subsets of entities in the plurality of entities.

Referring to Block 218, in some embodiments, each respective cluster in the plurality of clusters consists of a unique different subset of the plurality of entities.

In some embodiments, the clustering is done prior to implementation of the disclosed methods. For instance, in some embodiments the discrete attribute value dataset 120 already includes the cluster assignments for each entity (e.g., nucleus and/or probe spot) in the discrete attribute dataset.

Regardless of whether or not clustering is performed after retrieving the discrete attribute dataset or the discrete attribute value dataset already included cluster assignments for each entity, what is obtained is a corresponding cluster assignment in a plurality of clusters, of each respective entity in the plurality of entities of the discrete attribute value dataset. The corresponding cluster assignment (of each respective entity) is based, at least in part, on the corresponding plurality of discrete attribute values of the respective entity (e.g., the discrete attribute values that map to the respective entity in the discrete attribute value dataset), or a corresponding plurality of dimension reduction components derived, at least in part, from the corresponding plurality of discrete attribute values of the respective entity.

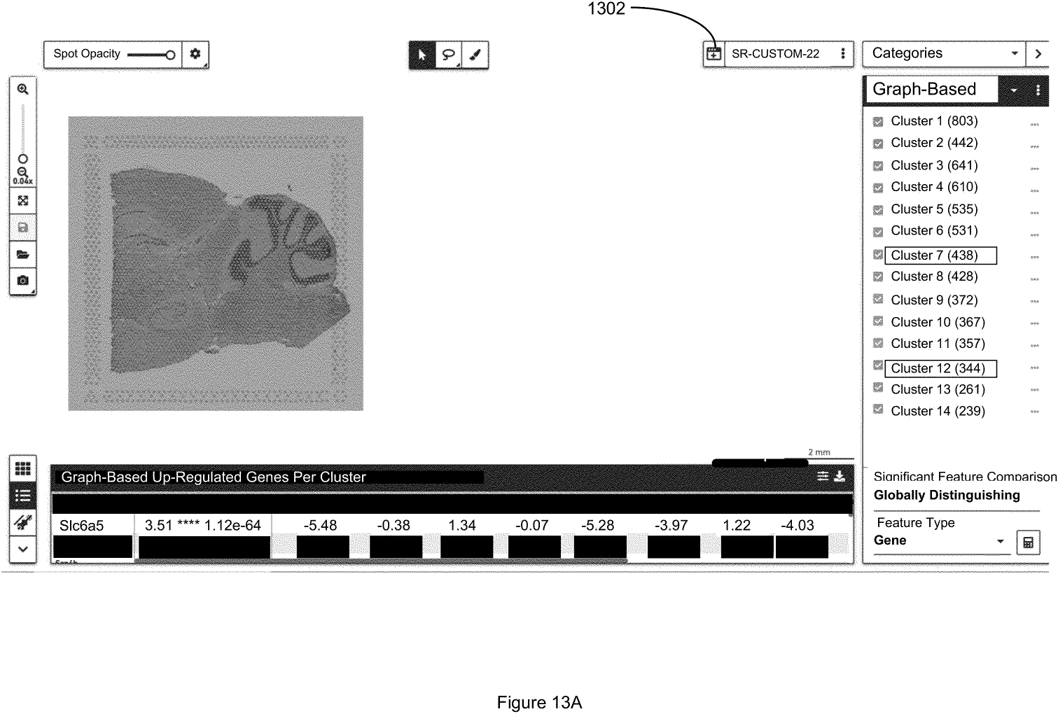

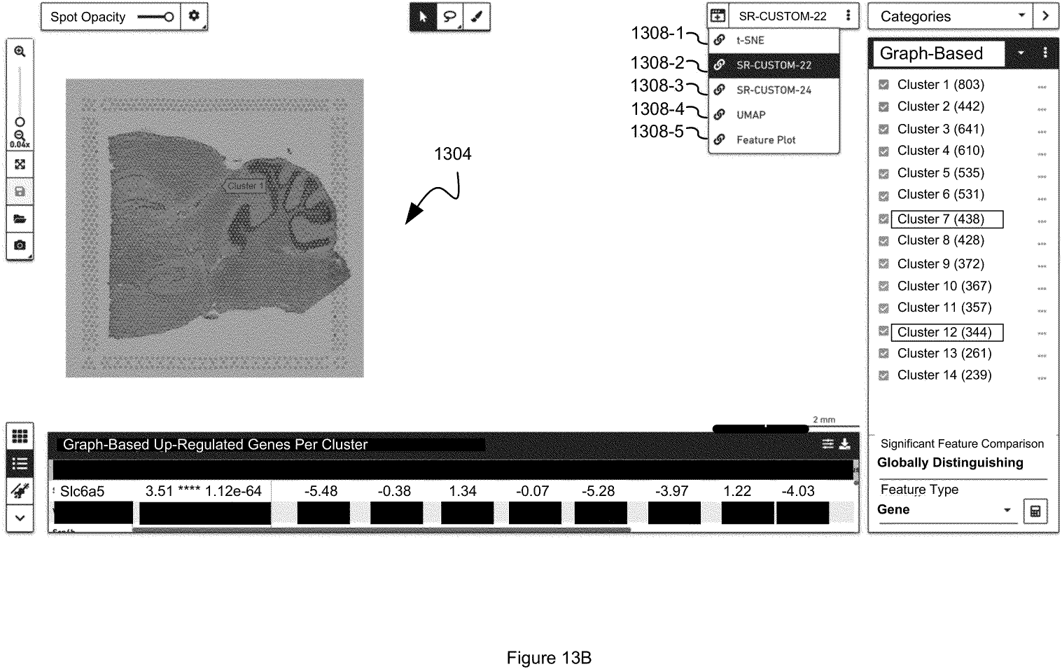

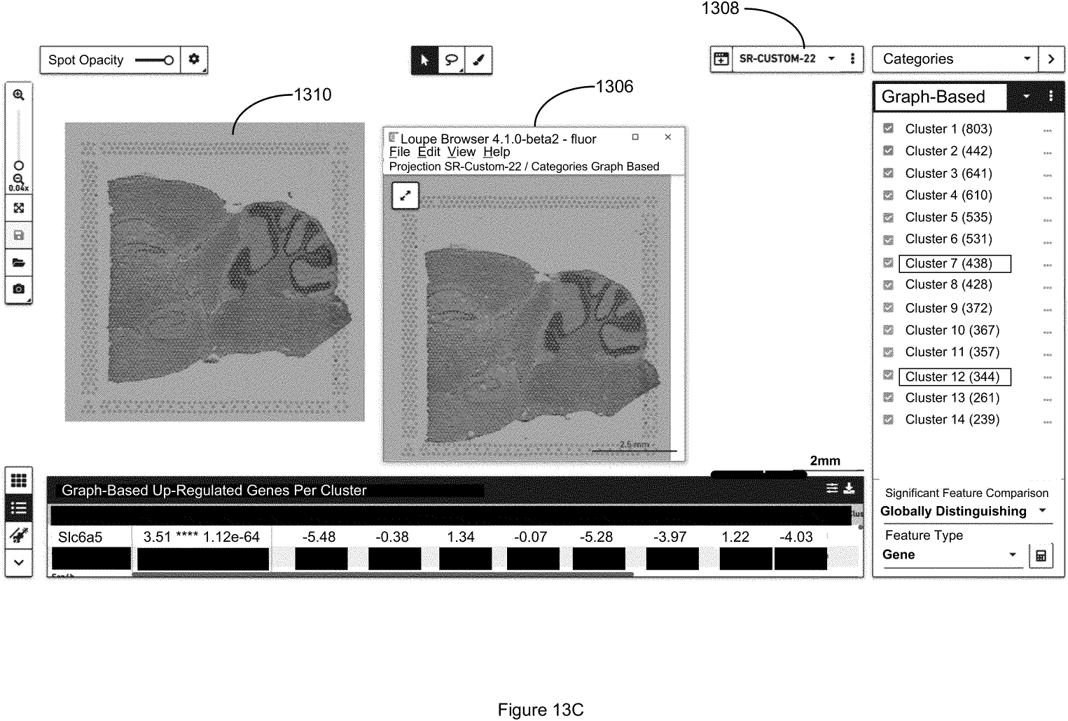

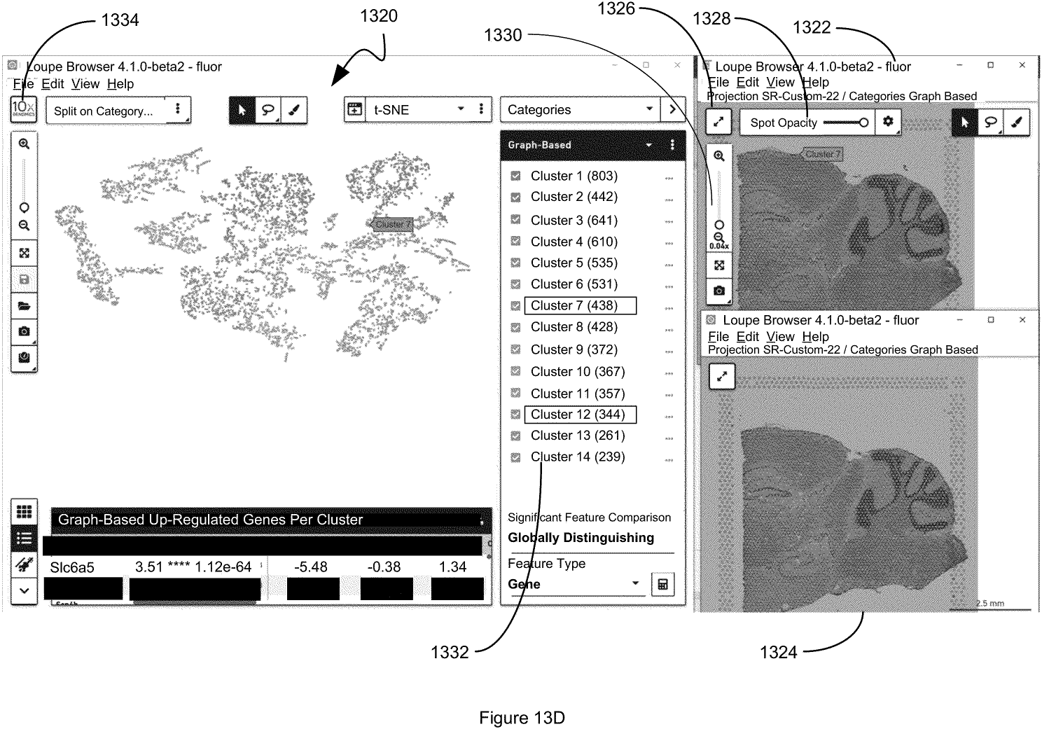

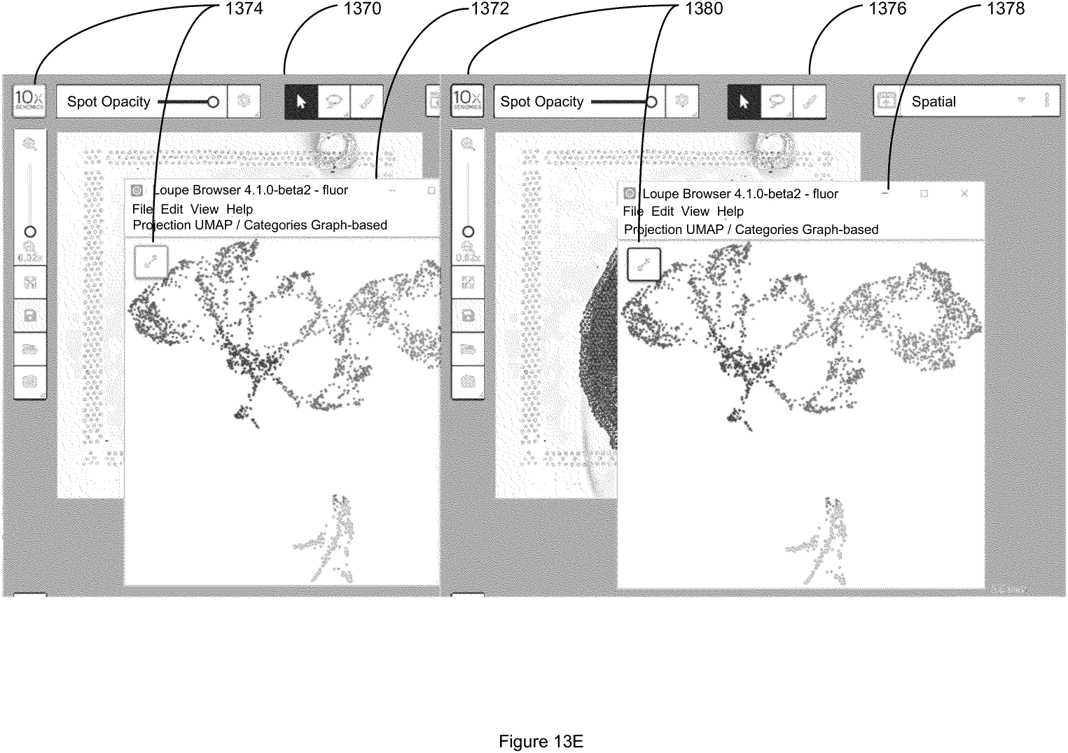

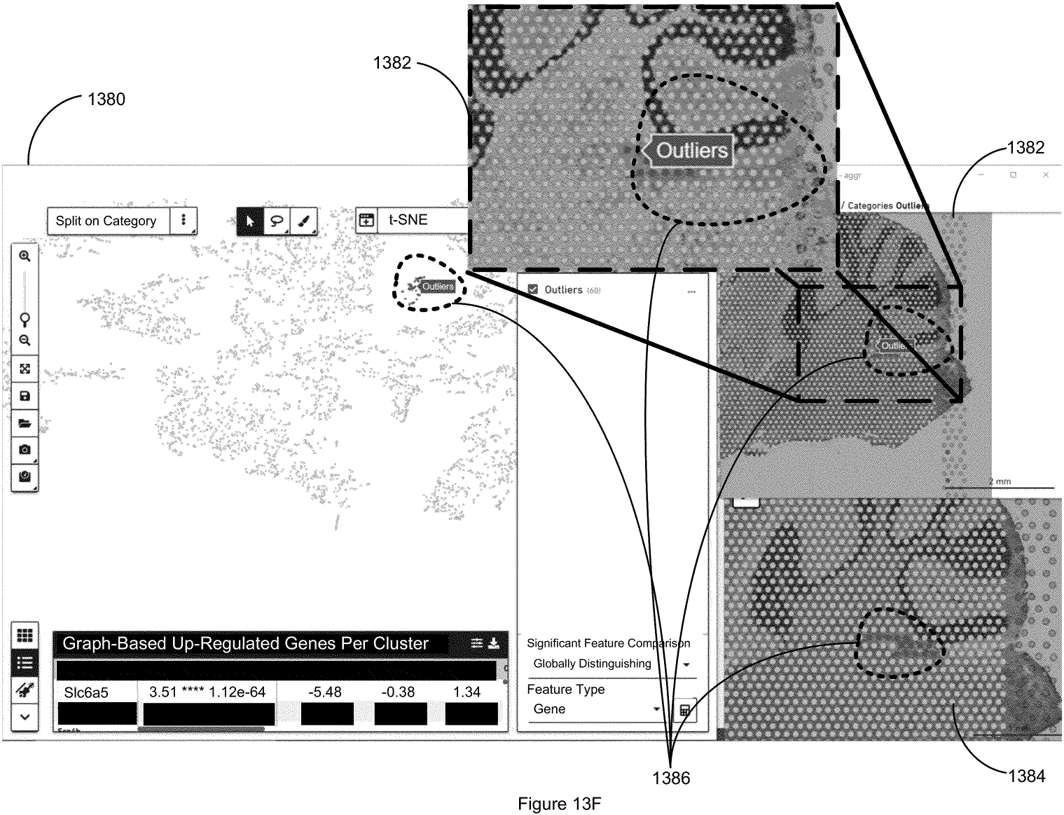

Referring to Block 220, in some embodiments, the method further comprises assigning each respective cluster in the plurality of clusters a different graphic or color code, and coloring each respective entity in the two-dimensional spatial arrangement of the plurality of entities in accordance with the different graphic or color code associated with the respective cluster corresponding to the respective entities. For instance, in some embodiments, as illustrated in FIGS. 13D and 16, each respective cluster in a plurality of clusters is indicated by a different color shading.

Referring to Block 222, in some embodiments, the clustering the discrete attribute value dataset comprises hierarchical clustering, agglomerative clustering using a nearest-neighbor algorithm, agglomerative clustering using a farthest-neighbor algorithm, agglomerative clustering using an average linkage algorithm, agglomerative clustering using a centroid algorithm, or agglomerative clustering using a sum-of-squares algorithm. Referring to Block 224, in some embodiments, the clustering the discrete attribute value dataset comprises application of a Louvain modularity algorithm, k-means clustering, a fuzzy k-means clustering algorithm, or Jarvis-Patrick clustering. See, Blondel et al., Jul. 25, 2008, “Fast unfolding of communities in large networks,” arXiv:0803.0476v2 [physical.coc-ph], which is hereby incorporated by reference. In some embodiments, the user can choose a clustering algorithm.

In an example embodiment, dimension reduction component values stored in the discrete attribute value dataset 120 that have been computed by the method of principal component analysis using the discrete attribute values 124 across the plurality of entities of the discrete attribute value dataset 120 are used to perform cluster visualization, as illustrated in FIG. 4.

Principal component analysis (PCA) is a mathematical procedure that reduces the number of correlated variables into fewer uncorrelated variables called “principal components.” The first principal component is selected such that it accounts for as much of the variability in the data as possible, and each succeeding component accounts for as much of the remaining variability as possible. The purpose of PCA is to discover or to reduce the dimensionality of the dataset, and to identify new meaningful underlying variables. PCA is accomplished by establishing actual data in a covariance matrix or a correlation matrix. The mathematical technique used in PCA is called eigen analysis: one solves for the eigenvalues and eigenvectors of a square symmetric matrix with sums of squares and cross products. The eigenvector associated with the largest eigenvalue has the same direction as the first principal component. The eigenvector associated with the second largest eigenvalue determines the direction of the second principal component. The sum of the eigenvalues equals the trace of the square matrix and the maximum number of eigenvectors equals the number of rows (or columns) of this matrix. See, for example, Duda, Hart, and Stork, Pattern Classification, Second Edition, John Wiley & Sons, Inc., NY, 2000, pp. 115-116, which is hereby incorporated by reference.

For clustering in accordance with one embodiment of the systems and methods of the present disclosure, regardless at what stage it is performed, consider the case in which each entity is associated with ten reference sequences 122. Each of the ten reference sequences represents a different analyte and/or feature under study, such as a different antibody, a different region of a reference genome, etc. In such instances, each entity can be expressed as a vector:

$${\vec{X}}_{10} = \{ {x}_{1} , {x}_{2} , {x}_{3} , {x}_{4} , {x}_{5} , {x}_{6} , {x}_{7} , {x}_{8} , {x}_{9} , {x}_{10} \}$$

where Xᵢ is the discrete attribute value 124 for the reference sequence i 124 associated with the entity in a given region of interest. Thus, consider the case where the discrete attribute dataset comprises a single spatial representation (e.g., image) and a single region of interest (e.g., of a biological sample) and there are one thousand entities in this single spatial arrangement. In this case, 1000 vectors are defined. Now, consider the case where the discrete attribute dataset comprises two spatial arrangements in each of three projections and there are one thousand entities in each of the spatial arrangements. In this case, 3 x 1000, or 3000 vectors are defined. Those entities that exhibit similar discrete attribute values across the set of reference sequences 122 of the dataset 102 will tend to cluster together. For instance, in the case where each entity corresponds to an individual nucleus (e.g., cell), the reference sequences 122 correspond to mRNA mapped to individual genes within such individual nuclei, and the discrete attribute values 124 are mRNA counts for such mRNA. It is the case in some embodiments that the discrete attribute value dataset 120 includes mRNA data from one or more entity types (classes, e.g., diseased state and non-diseased state), two or more entity types, or three or more entity types. In such instances, it is expected that entities of like type will tend to have like values for mRNA across the set of reference sequences (mRNA) and therefore cluster together. For instance, if the discrete attribute value dataset 120 includes class a: entities from subjects that have a disease, and class b: entities from subjects that do not have a disease, an ideal clustering classifier will cluster the discrete attribute value dataset 120 into two groups, with one cluster group uniquely representing class a and the other cluster group uniquely representing class b.

For clustering in accordance with another embodiment of the systems and methods of the present disclosure, regardless at what stage it is performed, consider the case in which each entity is associated with ten dimension reduction component values that collectively represent the variation in the discrete attribute values of a large number of reference sequences 122 of a given entity with respect to the discrete attribute values of corresponding reference sequences 122 of other entities in the dataset. This can be for a single spatial representation (e.g., spatial arrangement 125), across all or a subset of spatial arrangements in a single region of interest 121 (e.g., of a biological sample), or across all or a subset of the spatial arrangements in all or a subject of a plurality of regions of interest 125 in a discrete attribute value dataset 120. In such instances, each entity 126 can be expressed as a vector:

$${\vec{X}}_{10} = \{ {x}_{1} , {x}_{2} , {x}_{3} , {x}_{4} , {x}_{5} , {x}_{6} , {x}_{7} , {x}_{8} , {x}_{9} , {x}_{10} \}$$

where Xᵢ is the dimension reduction component value 164 i associated with the entity. Thus, if there are one thousand entities per spatial arrangement, a total of one thousand vectors are defined across the spatial arrangements. The vectors representing those entities that exhibit similar discrete attribute values across the set of dimension reduction component values 164 will tend to cluster together. It is the case, in some embodiments, that the discrete attribute value dataset 120 includes mRNA data from one or more entity types (e.g., diseased state and non-diseased state), two or more entity types, or three or more entity types. In such instances, it is expected that entities of like type will tend to have like values for mRNA across the set of reference sequences (mRNA) and therefore cluster together. For instance, if the discrete attribute value dataset 120 includes class a: entities from subjects that have a disease, and class b: entities from subjects that do not have a disease, an ideal clustering classifier will cluster the discrete attribute value dataset 120 into two groups, with one cluster group uniquely representing class a and the other cluster group uniquely representing class b.

Clustering is described on pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda 1973, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 216 of Duda 1973.

Once a method for measuring “similarity” or “dissimilarity” between points in a dataset has been selected, clustering requires a criterion function that measures the clustering quality of any partition of the data. Partitions of the dataset that extremize the criterion function are used to cluster the data. See page 217 of Duda 1973. Criterion functions are discussed in Section 6.8 of Duda 1973.

More recently, Duda et al., Pattern Classification, Second edition, John Wiley & Sons, Inc. New York, which is hereby incorporated by reference, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (Third Edition), Wiley, New York, N.Y.; and Backer, 1995, Computer-Assisted Reasoning in Cluster Analysis, Prentice Hall, Upper Saddle River, N.J. Particular exemplary clustering techniques that can be used in the systems and methods of the present disclosure to cluster a plurality of vectors, where each respective vector in the plurality of vectors comprises the discrete attribute values 124 across the reference sequences 122 of a corresponding entity (or dimension reduction components derived therefrom) includes, but is not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Thus, in some embodiments, the clustering module 152 clusters the discrete attribute value dataset 120 using the discrete attribute value 124 for each reference sequence 122 in the plurality of reference sequences for each respective entity in the plurality of entities, or dimension reduction component values 164 derived from the discrete attribute values 124, across one or more spatial arrangements in one or more regions of interest in the discrete attribute value dataset 120 thereby assigning each respective entity in the plurality of entities to a corresponding cluster 158 in a plurality of clusters and thereby assigning a cluster attribute value to each respective entity in the plurality of entities of each spatial arrangement used in the analysis.

Referring to Block 228, the clustering the discrete attribute value dataset comprises k-means clustering of the discrete attribute value dataset into a predetermined number of clusters. The goal of k-means clustering is to cluster the discrete attribute value dataset 120 based upon the dimension reduction components or the discrete attribute values of individual entities into K partitions. In some embodiments, the k-means algorithm computes like clusters of entities from the higher dimensional data (the set of dimension reduction component values) and then after some resolution, the k-means clustering tries to minimize error. In this way, the k-means clustering provides cluster assignments 158, which are recorded in the discrete attribute value dataset 120.

In some embodiments, K is a number between 2 and 50 inclusive. In some embodiments, the number K is set to a predetermined number such as 10. In some embodiments, the number K is optimized for a particular discrete attribute value dataset 120. In some embodiments, the number Kis 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30. In some embodiments, the number K is at least 30, at least 35, at least 40, at least 45, at least 50, at least 60, at least 70, at least 80, at least 90, or at least 100.

Referring to Block 230, the clustering the discrete attribute value dataset comprises k-means clustering of the discrete attribute value dataset into a number of clusters, wherein the number is acquired based on user input. In some embodiments, a user sets the number K using the visualization module 119.

FIG. 4 illustrates an instance in which the multichannel-aggr dataset 120, constituting data from a plurality of entities (e.g., probe spots) has been clustered into eleven clusters 158. In some embodiments, for k-means clustering, the user selects in advance how many clusters the clustering algorithm will compute prior to clustering. In some embodiments, no predetermined number of clusters is selected. Instead, clustering is performed until predetermined convergence criteria are achieved. In embodiments where a predetermined number of clusters is determined, k-means clustering of the present disclosure is then initialized with K cluster centers µₗ, ..., µ_{K} randomly initialized in two-dimensional space. As discussed above, for each respective entity 126 i in the dataset, a vector Xᵢ is constructed of each dimension reduction component value 164 associated with the respective entity 126. In the case where K is equal to 10, ten such vectors

$$\vec{\text{X}}$$

are selected to be the centers of the ten clusters. Then, each remaining vector

$${\vec{\text{X}}}_{i ,}$$

corresponding to the entities 126 that were not selected to be cluster centers, is assigned to its closest cluster center:

$${C}_{k} = \left\{n : k = arg {}_{k}^{m i n} {\vec{X}}_{i} - {μ}_{k} {2}^{}\right\}$$

where Cₖ is the set of examples closest to µₖ using the objective function:

$$J \left(μ , r\right) = {\sum }_{n = 1}^{N} {\sum }_{k = 1}^{K} {r}_{n k} {\vec{X}}_{i} - {μ}_{k} {2}^{}$$

where µₗ, ..., µ_{K} are the K cluster centers and rₙₖ ∈ {0, 1} is an indicator denoting whether an entity 126 Xᵢ belongs to a cluster k. Then, new cluster centers µₖ are recomputed (mean / centroid of the set Cₖ):

$${μ}_{k} = \frac{1}{{C}_{k}} {\sum }_{n ∈ {C}_{k}} {\vec{X}}_{i}$$

Then, all vectors Xᵢ, corresponding to the entities 126 are assigned to the closest updated cluster centers as before. This is repeated while not converged. Any one of a number of convergence criteria can be used. One possible convergence criterion is that the cluster centers do not change when recomputed. The k-means clustering computes a score for each respective entity 126 that takes into account the distance between the respective entity and the centroid of the cluster 158 that the respective entity has been assigned. In some embodiments, this score is stored as the cluster attribute value 160 for the entity 126.

Once the clusters are identified, as illustrated in FIG. 4, individual clusters can be selected to display. For instance, referring to FIG. 4, affordances 440 are individually selected or deselected to display or remove from the display the corresponding cluster 158.

As illustrated in FIG. 4, in accordance with the systems and methods of the present disclosure, in typical embodiments, each respective cluster 158 in the plurality of clusters consists of a unique different subset of the second plurality of entities 126. Moreover, because in typical embodiments the discrete attribute value dataset 120 is too large to load into the non-persistent memory 111, in typical embodiments this clustering loads less than the entirety of the discrete attribute value dataset 120 into the non-persistent memory 111 at any given time during the clustering. For instance, in embodiments where the discrete attribute value dataset 120 has been compressed using bgzf, only a subset of the blocks of the discrete attribute value dataset 120 are loaded into non-persistent memory during the clustering of the discrete attribute value dataset 120. Once one subset of the blocks of the discrete attribute value dataset 120 have been loaded from persistent memory 112 into non-persistent memory 111 and processed in accordance with the clustering algorithm (e.g., k-means clustering), the subset of blocks of data is discarded from non-persistent memory 111 and a different subset of blocks of the discrete attribute value dataset 120 are loaded from persistent memory 112 into non-persistent memory 111 and processed in accordance with the clustering algorithm of the clustering module 152.

As described above, in some embodiments, a two-dimensional spatial arrangement refers to an image indicating the two-dimensional positions of spatial analyte data within a given frame of reference (e.g., an image of a biological sample). Moreover, in some embodiments, an image comprises a plurality of pixels, e.g., arranged in an array (see, e.g., Definitions: Imaging and Images).

Accordingly, referring to Block 232, in some embodiments, the two-dimensional spatial arrangement of the plurality of entities on the display comprises 1,000,000 pixel values. In some embodiments, each two-dimensional spatial arrangement comprises at least 10,000 pixel values, at least 20,000 pixel values, at least 50,000 pixel values, at least 100,000 pixel values, at least 200,000 pixel values, at least 300,000 pixel values, at least 500,000 pixel values, at least 1 million pixel values, at least 2 million pixel values, at least 3 million pixel values, at least 4 million pixel values, at least 5 million pixel values, at least 6 million pixel values, at least 7 million pixel values, at least 8 million pixel values, at least 9 million pixel values, at least 10 million pixel values, or at least 15 million pixel values.

Referring to Block 234, the method includes displaying the two-dimensional spatial arrangement of the plurality of entities on the display.

In some embodiments, a discrete attribute value dataset 120 (e.g., a .cloupe file) includes spatial information (e.g., additional information beyond gene expression data, etc.) for a plurality of entities (e.g., nuclei and/or probe spots). In some embodiments, the discrete attribute value dataset 120 comprises at least a) a spatial feature-barcode matrix for the relative expression of genomic reference sequences at each entity, and b) the coordinates, in image pixel units, of the centers of the entities for each barcode in the feature-barcode matrix. In some embodiments, such discrete attribute value dataset 120 contain multiple projections of the data. Examples of such projections include mathematical projections in t-SNE two-dimensional coordinate space and a UMAP two-dimensional coordinate space (e.g., as described above), projections of entity coordinates (e.g., based on the respective barcode for each entity), and/or projections of fiduciary coordinates (e.g., based on one or more spatial fiducials). A respective set of entity coordinates correspond to the center of the corresponding entity in pixel units. Some such projections further include the diameter of each entity in pixel units.

In an example embodiment, opening a discrete attribute value dataset 120 (e.g., .cloupe file) with spatial information comprises opening a spatial analysis view panel 704 within the visualization module (see FIG. 7). In some embodiments, the visualization module is, in many aspects, similar to the browser described in U.S. Pat. Publication No. US 2021-0062272, entitled “Systems and Methods for Using the Spatial Distribution of Haplotypes to Determine a Biological Condition,” which is hereby incorporated by reference. The spatial analysis view panel (which is selected, e.g., using the “Spatial” option 702) enables visualization of gene expression in the context of tissue images. In some embodiments, each entity is displayed overlaid on an original image, and each entity is spatially oriented with respect to every other entity in the plurality of entities. Further, and as described below, each entity is, in some embodiments, annotated (e.g., via color) to indicate gene expression, membership in a cluster (e.g., as described above), and other information.

In some embodiments, a respective discrete attribute value dataset 120 (e.g., .cloupe file) with associated image information includes one or more corresponding image files (e.g., separate from the respective discrete attribute value dataset 120 itself), and opening the respective discrete attribute value dataset 120 does not automatically load the corresponding image files. In other words, in some embodiments, in contrast to what is illustrated in FIG. 1B, spatial arrangements 125 are stored external to the discrete attribute value dataset 120 itself. In some embodiments, after a respective discrete attribute value dataset 120 is opened, a user request to view a corresponding spatial arrangement 125 (or set of spatial arrangements of a region of interest 121) results in opening spatial analysis view panel 704 within the visualization module and image processing and tiling as required.

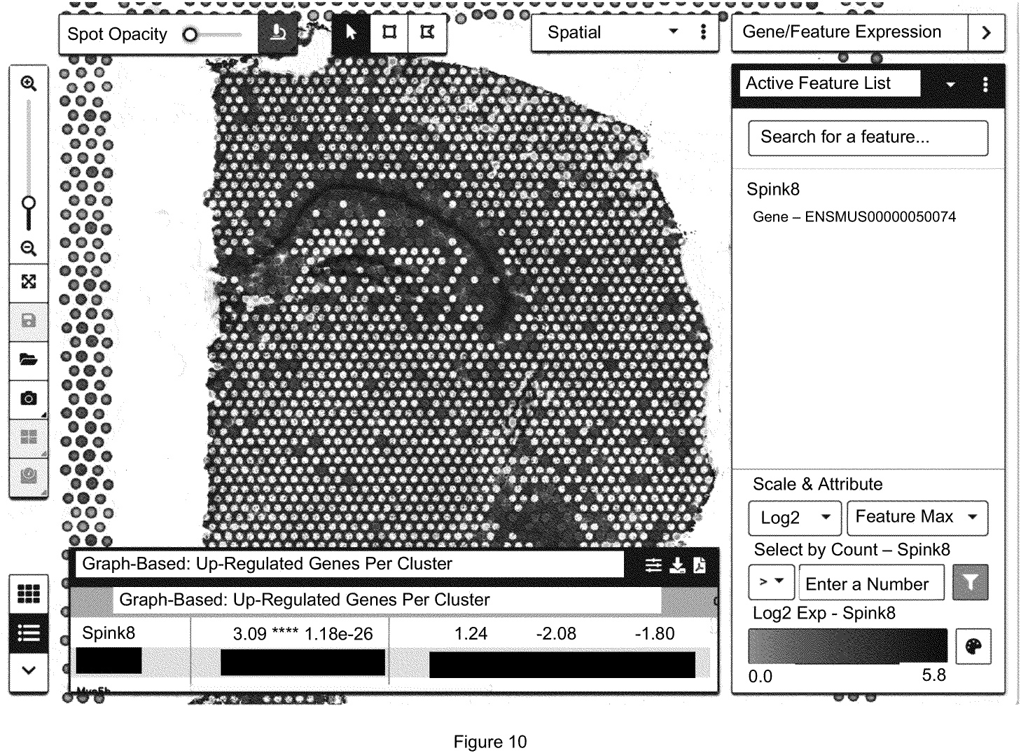

In some embodiments, each discrete attribute file dataset 120 includes information identifying one or more significant features (e.g., gene expression, feature barcode analyte count, etc.) corresponding to each cluster in the plurality of clusters. For example, in FIG. 10, a user has selected a single gene (e.g., ‘Spink8’). The selection of Spink8 results in display of the expression of this gene within the spatial arrangement (e.g., for each entity in the plurality of displayed entities). The expression of this gene is clearly highlighted in the resulting spatial arrangement. Thus, users can clearly view the correlation of the expression of particular features overlaid on the underlying image file.

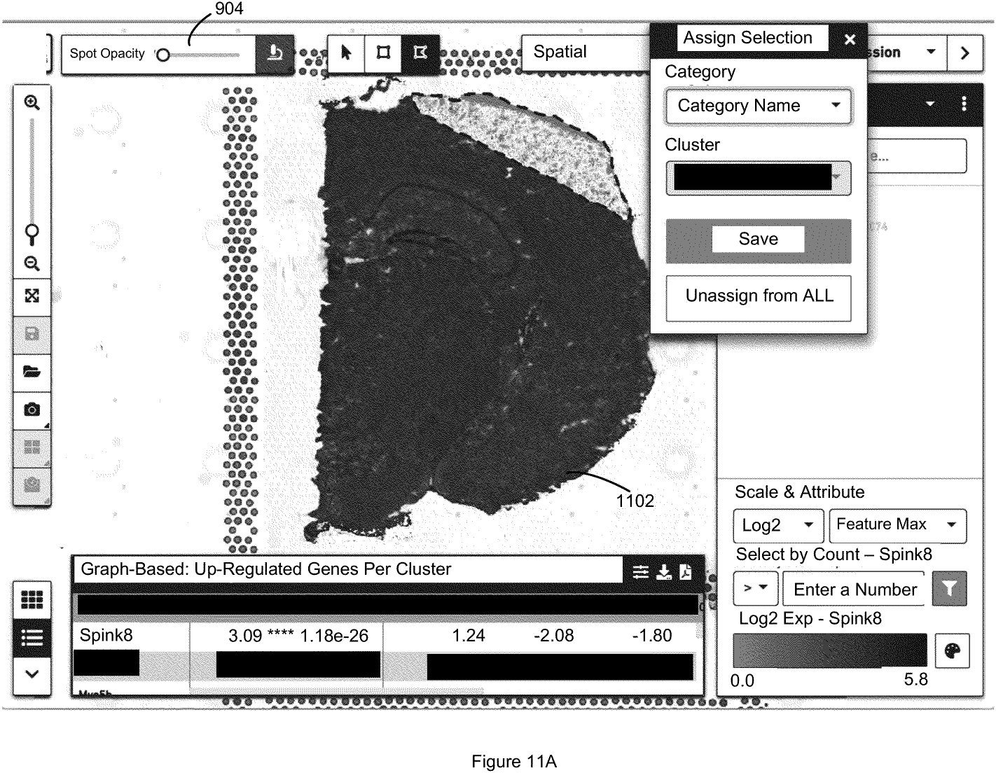

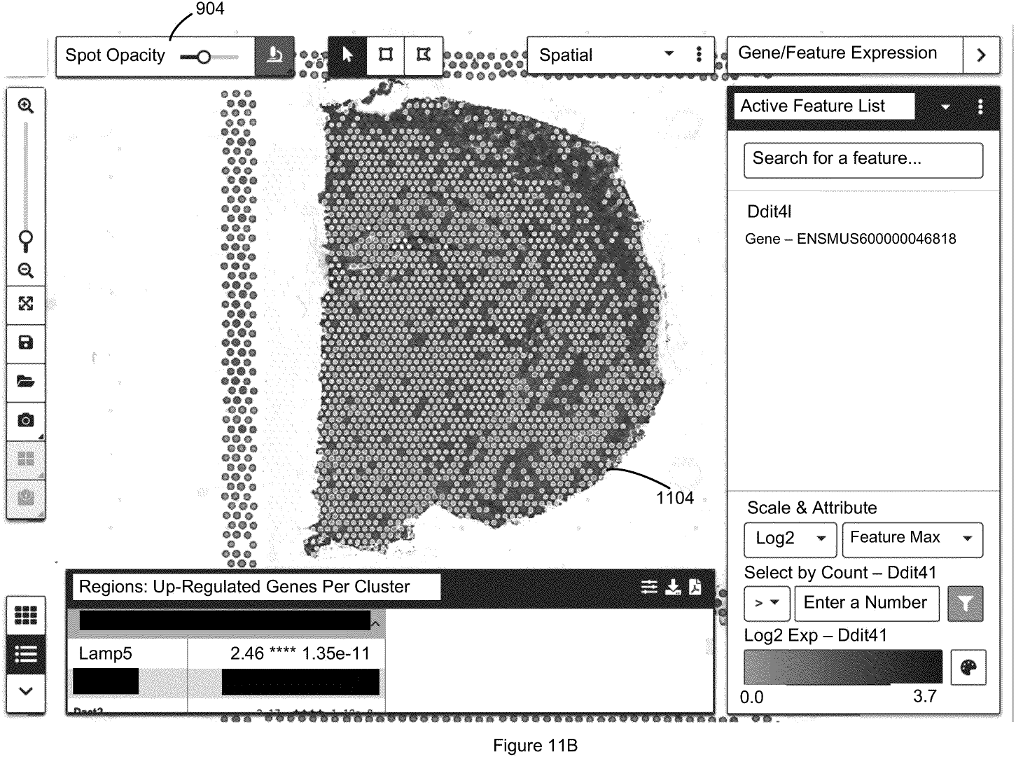

In some embodiments, low entity opacity (e.g., as shown in FIG. 11A in entity opacity bar 904) permits visualization of an underlying image file (or set of images files) without any interaction with feature display, which is desirable to view aspects of the tissue itself (e.g., region 1102 represents the tissue sample). FIG. 11B illustrates increased entity opacity (e.g., as seen in entity opacity bar 904), combined with feature information (e.g., here gene expression of ‘Ddit41’). For example, a plurality of entities (e.g., probe spots) can be seen in region 1104 in FIG. 11B. Switching between the views in FIGS. 11A and 11B enables discovery of patterns of gene expression alongside tissue features in an interactive manner.

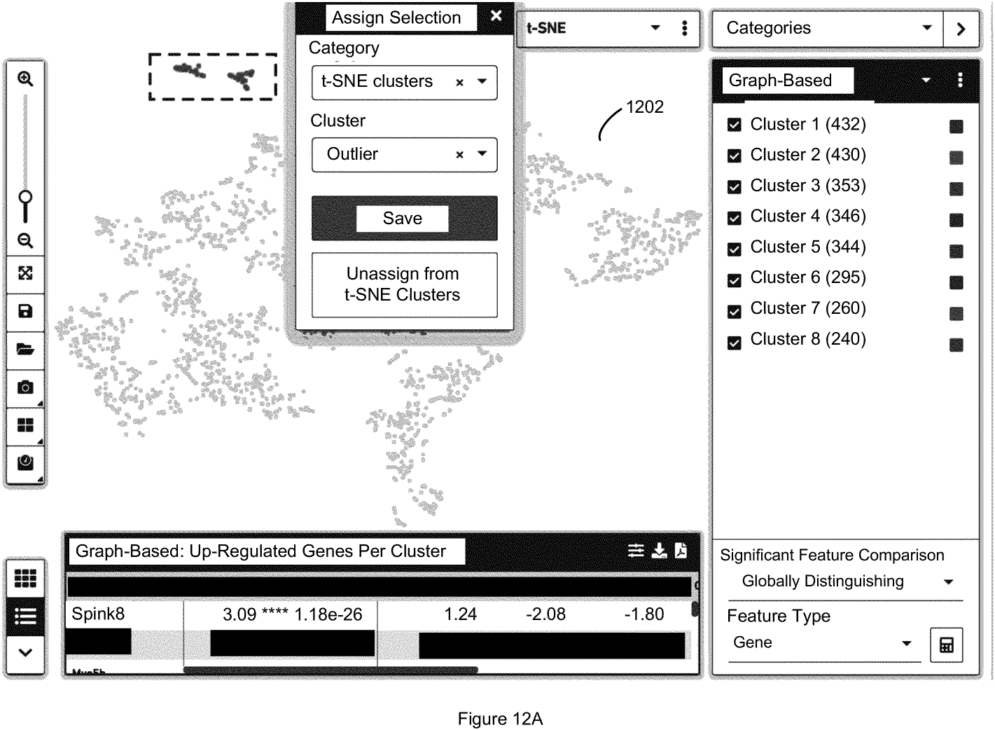

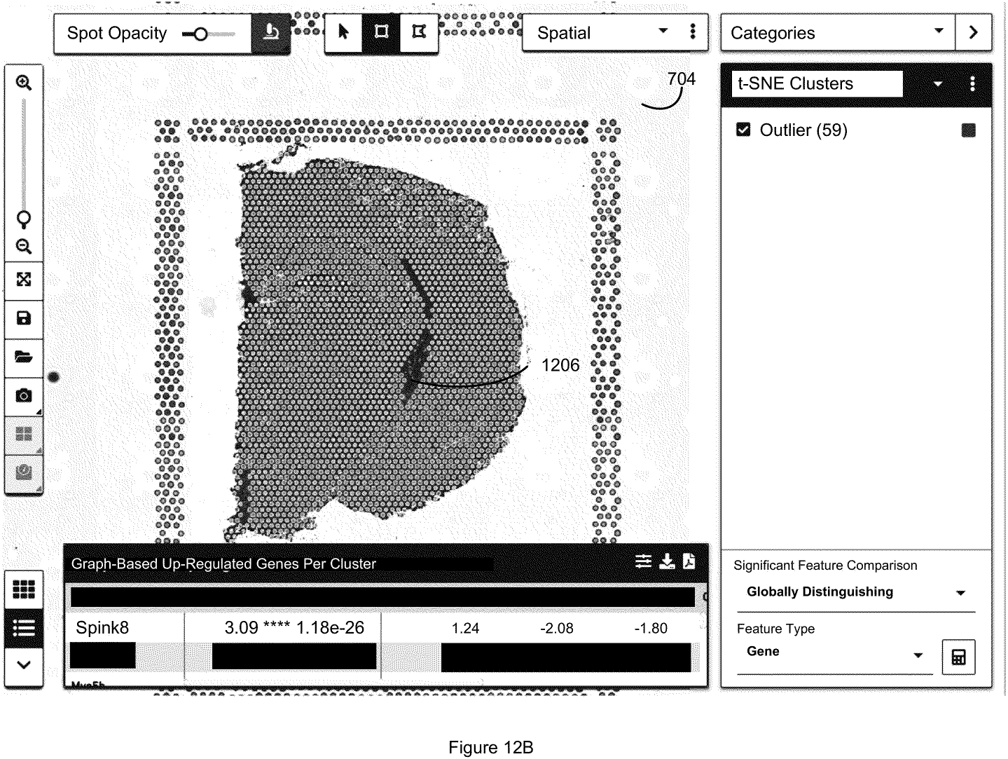

In some embodiments, as shown in FIG. 12A, a projection of entity expression information into t-SNE space is provided. In some embodiments, a projection of entity expression into UMAP space can also be shown. Such projections illustrate one or more clusters. As described above with regard to other embodiments of the present disclosure, a single cluster (e.g., ‘Outliers’) can be selected and displayed in either the t-SNE projection space (e.g., region 1202 of FIG. 12A) or in the spatial analysis view panel 704 (e.g., region 1206 in FIG. 12B).

In some embodiments, image display, manipulation, and export are performed as described in U.S. Pat. Publication No. US 2018-0052594, entitled “Providing Graphical Indication of Label Boundaries in Digital Maps” or U.S. Patent Publication No. US 2018-0052593, entitled “Providing Visual Selection of Map Data for a Digital Map”, which are hereby incorporated by reference.

In some embodiments, the displaying the two-dimensional spatial arrangement of the plurality of entities on the display comprises submitting one or more discrete attribute values to a graphical processing unit (e.g., a graphics card).

In some embodiments, the displaying the two-dimensional spatial arrangement of the plurality of entities on the display comprises submitting one or more discrete attribute values to a rendering library. In some embodiments, the rendering library is Plotly. See, for example, Plotly Technologies Inc. Collaborative data science. Montreal, QC, 2015. In some embodiments, the rendering library is DeckGL (available on the Internet at deck.gl).

In some embodiments, the two-dimensional spatial arrangement of the plurality of entities is displayed in grayscale. In some embodiments, the two-dimensional spatial arrangement of the plurality of entities comprises a plurality of spatial image layers, where each respective layer is displayed in color and where the plurality of spatial image layers is overlaid in a stack of layers.

In some embodiments, the two-dimensional spatial arrangement of the plurality of entities is displayed as a plurality of tiles, where each tile in the plurality of tiles is loaded onto the display independently. In some embodiments, the two-dimensional spatial arrangement of the plurality of entities is loaded in its entirety to the display.

In some embodiments, the two-dimensional spatial arrangement of the plurality of entities comprises a plurality of instances of spatial projections, where each spatial projection is an instance of an image of the two-dimensional spatial arrangement or a representation thereof (e.g., an analysis, chart, graph, etc.).

For instance, FIGS. 4, 5, 7 and 8 illustrate a single window that displays a region of interest 121, where the region of interest 121 consists of a single two-dimensional spatial representation (e.g., spatial arrangement 125). As disclosed above, in some embodiments a region of interest 121 comprises several spatial arrangements (e.g., several two-dimensional spatial representations can be obtained to represent the single region of interest 121). In such instances, a user is able to use the visualization tool (e.g., viewer) illustrated in FIG. 7 to concurrently view all the spatial arrangements 125 of the single region of interest 121 overlayed on each other. That is, the viewer illustrated in FIG. 7 concurrently displays all the spatial arrangements 125 of the single region of interest 121 overlayed on each other. In some such embodiments, the user is able to selectively un-display some of the spatial arrangements 125 of the single region of interest 121. That is, any combination of the spatial arrangements of a region of interest, superimposed on each other, can be concurrently viewed in the viewer. Moreover, the user can initiate more than one viewer illustrated in FIG. 7 onto the screen at the same time, and each such viewer can display all or a subset of the spatial arrangements of a corresponding region of interest 121 on the display.

### Subset Selection

Referring to Block 236, the method further comprises receiving a user selection of a subset of the two-dimensional spatial arrangement on the display.

For instance, in some embodiments, referring to Block 238, the receiving the user selection of the subset of the two-dimensional spatial arrangement on the display comprises obtaining a closed form shape drawn by a user on the display that is within or overlaps the two-dimensional spatial arrangement. In some embodiments, the closed form shape is a geometric shape (e.g., rectangle, circle, triangle, etc.). In some embodiments, the closed form shape is a free-form shape (e.g., generated using a free-form selection tool).

In some embodiments, the user selection of the subset of the two-dimensional spatial arrangement comprises including or excluding all of the pixels of the displayed two-dimensional spatial arrangement selected by the user. Accordingly, referring to Block 242, in some embodiments, the subset is each entity in the plurality of entities that is outside the closed form shape. Alternatively, referring to Block 244, in some embodiments, the subset is each entity in the plurality of entities that is inside the closed form shape.

In some embodiments, the user selection comprises clicking or highlighting one or more pixels of the two-dimensional spatial arrangement on the display, thereby selecting the regions of the two-dimensional spatial arrangement containing the selected pixels.

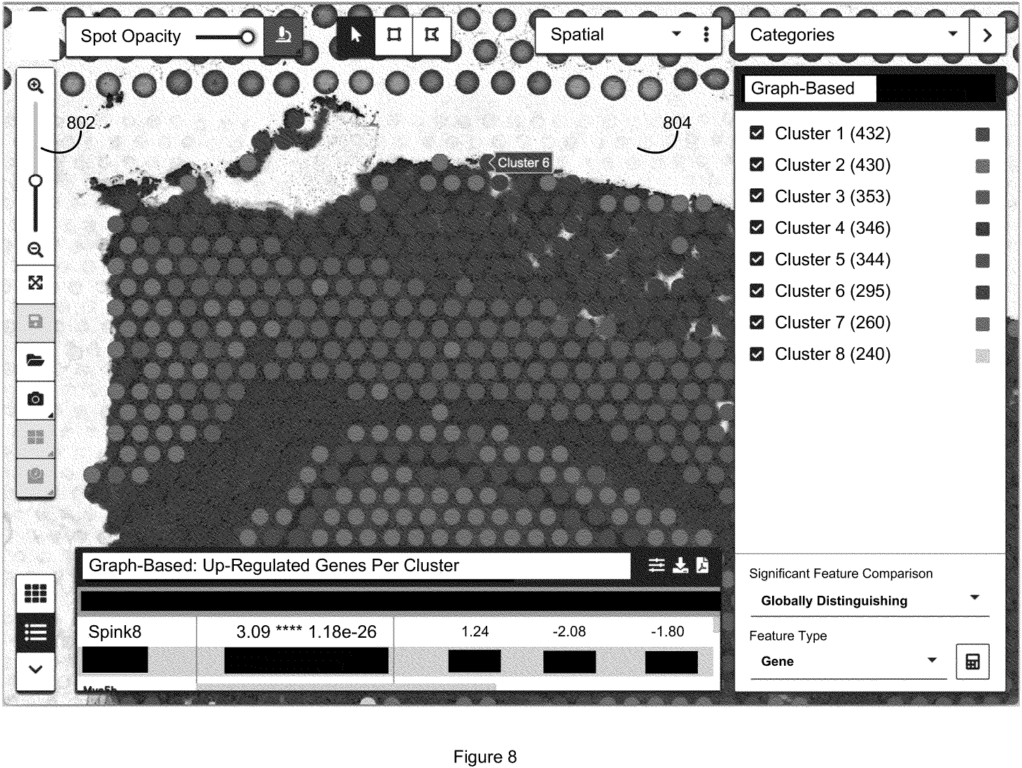

In some embodiments, a respective user selection (e.g., a zooming input) results in zooming the spatial analysis view into a region of the tissue (see e.g., FIG. 8, which illustrates a zoomed-in region of FIG. 7). In some embodiments, the user selection comprises adjusting the zoom slider 802 (e.g., see the difference in the sizes of the plurality of probe spots between panels 704 and 804) and loading the appropriate tile corresponding to the desired location on the spatial arrangement. For discrete attribute value datasets 120 that have multiple spatial arrangements in a region of interest (e.g., fluorescent, multichannel datasets), spatial arrangement tiles are retrieved based on the zoom level (of zoom slider 802) and position of the viewer with tiles retrieved for each active spatial arrangement concurrently. The active spatial arrangements are then composited together. In some embodiments, the displayed size of each entity (e.g., nucleus and/or probe spot) in the plurality of entities is dynamically altered after the adjustment of the zoom slider 802 is complete, to always reflect the approximate location and diameter of the entities relative to the original biological sample (see panel 804 in FIG. 8). In some embodiments, a panning input and/or a zooming user input will trigger the loading of the appropriate tile. This enables visualization of the spatial arrangement at much higher resolution without overloading visualization module 119 memory with off-canvas spatial arrangement data (e.g., with portions of the discrete attribute value dataset that are not being presented to the user).

In some embodiments, panning and zooming user inputs also trigger loading of a respective tile corresponding to a desired location in the spatial arrangement. Thus, a spatial arrangement (or set of spatial arrangements) can be viewed at much higher resolutions without overloading visualization module 119 memory with off-canvas spatial arrangement data.

In some embodiments, one or more spatial arrangement settings can be adjusted. For example, in FIG. 9A, selection of a spatial arrangement settings affordance (e.g., microscope icon 902) provides for user selection of one or more spatial arrangement settings (e.g., brightness, contrast, saturation, rotation, etc.). In some embodiments, a user can flip the spatial arrangement horizontally, rotate it to its natural orientation via slider or by entering the number of degrees of rotation, and adjust brightness and saturation of the spatial arrangement. In some embodiments, to see the underlying details of the tissue, a user makes a selection to adjust opacity. For example, in FIG. 9A, an opacity slider 904 provides for increasing or decreasing the transparency of the plurality of displayed entities. This permits a user to explore and determine an appropriate balance of feature information (e.g., as illustrated by the entities) combined with underlying spatial arrangement information, as described above.

Referring to Block 246, the method further includes determining each entity in the plurality of entities that is a member of the subset using the k-dimensional binary search tree, thereby identifying a subset of entities in the plurality of entities.

Generally, k-dimensional trees (k-d trees) are space-partitioning data structures used for organizing points in a k-dimensional space within nodes of a tree, where each node contains one point. K-d trees subdivide data at each recursive level of the tree, each parent node splitting its respective space into a left subspace and a right subspace, where the dimension of splitting the left and right subspaces relative to each other is dependent on the level of the tree. Location of a respective point within the data structure (e.g., point selection) can be performed by recursively determining whether the desired point is located to the left or the right subspace of each parent node, for each respective level of the tree, until the desired point is found. By reducing the searchable area of the tree by 50% at each level of the tree, point selection is simplified and the speed with which selection occurs is increased. See, for instance, Brown, “Building a Balanced k-d Tree in O(kn log n) Time,” Journal of Computer Graphics Techniques Vol. 4, No. 1, 2015, which is hereby incorporated herein by reference in its entirety. In some such embodiments, each respective point in the k-d tree is a respective entity. In some such embodiments, each respective point in the k-d tree is a respective nucleus. In some embodiments, each respective point in the k-d tree is a respective probe spot. In some embodiments, subset selection is performed for a subset of entities. In some such embodiments, subset selection is performed for entities (e.g., for nuclei and/or probe spots) using the k-dimensional binary search tree.

In some embodiments, the determining each point (e.g., each entity in the plurality of entities) that is a member of the subset using the k-dimensional binary search tree further comprises performing a translation between coordinate systems for each selected point (e.g., entity in the subset of selected entities).

For instance, in some embodiments, the two-dimensional spatial arrangement of the plurality of entities can be visualized on a display using a first spatial projection (e.g., a first display, a first window, a first graphical representation of an analysis of the discrete attribute value dataset corresponding to the plurality of entities, and/or a representation thereof). Each respective entity in the plurality of entities has a corresponding first coordinate position within the first spatial projection. In some embodiments, the two-dimensional spatial arrangement of the plurality of entities can be further visualized on a display using a second spatial projection (e.g., a display, a window, a graphical representation of an analysis of the discrete attribute value dataset corresponding to the plurality of entities, and/or a representation thereof other than the first spatial projection), where each respective entity in the plurality of entities has a corresponding second coordinate position within the second spatial projection. In some such embodiments, selection of each respective entity in the plurality of entities comprises determining the coordinates of the respective entity in the first spatial projection, performing a coordinate translation to determine the coordinates of the respective entity in the second spatial projection, and selecting the respective entity in both the first spatial projection and the second spatial projection.

In another example, in some embodiments, the two-dimensional spatial arrangement of the plurality of entities can be visualized on a display using a first spatial projection (e.g., a first display, a first window, a first graphical representation of an analysis of the discrete attribute value dataset corresponding to the plurality of entities, and/or a representation thereof), where each respective entity in the plurality of entities has a corresponding first coordinate position within the first spatial projection. Moreover, each respective entity in the plurality of entities has a corresponding global two-dimensional position, where the global two-dimensional position is considered to be the “absolute” position of the respective entity in the two-dimensional spatial arrangement. For instance, an absolute two-dimensional position can be a position (e.g., a two-dimensional and/or coordinate position) of the respective entity within a frame of reference relative to the original spatial context of the biological sample. In some embodiments, an absolute two-dimensional position can be a position (e.g., a two-dimensional and/or coordinate position) of the respective entity within a frame of reference relative to a substrate (e.g., one or more fiducial marks). In some embodiments, an absolute two-dimensional position can be a position (e.g., a two-dimensional and/or coordinate position) of the respective entity within a frame of reference relative to a designated coordinate point (e.g., a user selected point and/or a reference entity within the plurality of entities). In some such embodiments, selection of each respective entity in the plurality of entities comprises determining the coordinates of the respective entity in the first spatial projection, performing a coordinate translation to determine the absolute two-dimensional position of the respective entity, and selecting the respective entity based on the determined absolute two-dimensional position.

In an example embodiment, a respective point (e.g., an entity) is located at the position (5,5) in a first projection (e.g., a t-SNE projection) having an origin located at the position (0,0). Panning the origin of the first projection on the display (e.g., by a user interaction) can adjust the relative position of the respective point such that the origin of the display is located at the position (4,4), thereby adjusting the position of the respective point to (1,1). In some such embodiments, a coordinate translation is performed to determine the absolute two-dimensional position of the respective point and/or to determine the position of the respective point after the adjustment relative to before the adjustment, such that the point can be accurately located and selected.

### Category Assignment

Referring to Block 248, the method further includes assigning each entity in the subset of entities to a user provided category.

In some embodiments, the user provided category is a tissue type, an organ type, a species, an assay conditions, a clinical condition (e.g., healthy or diseased), a patient characteristic, a demographic, a cluster membership, an annotation, a sample preparation label (e.g., a stain), an analyte label (e.g., gene identifier), and/or any combination thereof.

Accordingly, the present disclosure provides situations in which method includes evaluation of multiple classes and/or categories of biological sample (e.g., tissue). That is, situations in which each such sample consists of first discrete attribute values 124 for each respective reference sequence 122 (e.g., mRNA that map to a particular gene in a plurality of genes) in each entity associated with a first condition (therefore representing a first class 172), second discrete attribute values 124 for each respective reference sequence 122 in each entity associated with a second condition (therefore representing a second class 172), and so forth, where each such class 172 refers to a different tissue type, different tissue condition (e.g., tumor versus healthy) a different organ type, a different species, or different assay conditions (e.g., dye type) or any of the forgoing. In some embodiments, the discrete attribute value dataset 120 contains data for two or more such classes, three or more such classes, four or more such classes, five or more such classes, ten or more such classes 172, or 100 or more such classes 172.

In some embodiments, the user provided category is selected (e.g., provided) from a plurality of categories. In some embodiments, the plurality of categories includes at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 500, or at least 1000 categories. In some embodiments, the plurality of categories includes no more than 5000, no more than 1000, no more than 500, no more than 100, no more than 50, or no more than 10 categories. In some embodiments, the plurality of categories includes from 2 to 10, from 5 to 20, from 10 to 50, from 8 to 100, or from 30 to 500 categories. In some embodiments, the plurality of categories falls within another range starting no lower than 2 categories and ending no higher than 5000 categories.

In some embodiments, there are multiple classes 172 of entities. In some embodiments, each entity contains multiple classes. In some embodiments, only a subset of the entities belong to one class (category) while other entities belong to a different category. In some embodiments, each such sample comprises first discrete attribute values 124 for each respective reference sequence 122 (e.g., mRNA that map to a particular gene in a plurality of genes) in each entity in a first plurality of entities under a first condition (therefore representing a first class 172), second discrete attribute values 124 for each respective reference sequence 122 in each entity in a second plurality of different entities under a second condition (therefore representing a second class 172), and so forth. In other situations, each such sample comprises first discrete attribute values 124 for each respective reference sequence 122 (e.g., mRNA that map to a particular gene in a plurality of genes) in each entity in a first plurality of entities of a first type (a first class 172), second discrete attribute values 124 for each respective reference sequence 122 in each entity in a second plurality of entities of a second type (a second class 172), and so forth, where each such class 172 refers to a different tissue type, a different organ type, a different species, or different assay conditions or any of the foregoing. In some embodiments, the discrete attribute value dataset 120 contains data for entities from two or more such classes, three or more such classes, four or more such classes, five or more such classes, ten or more such classes 172, or 100 or more such classes 172.

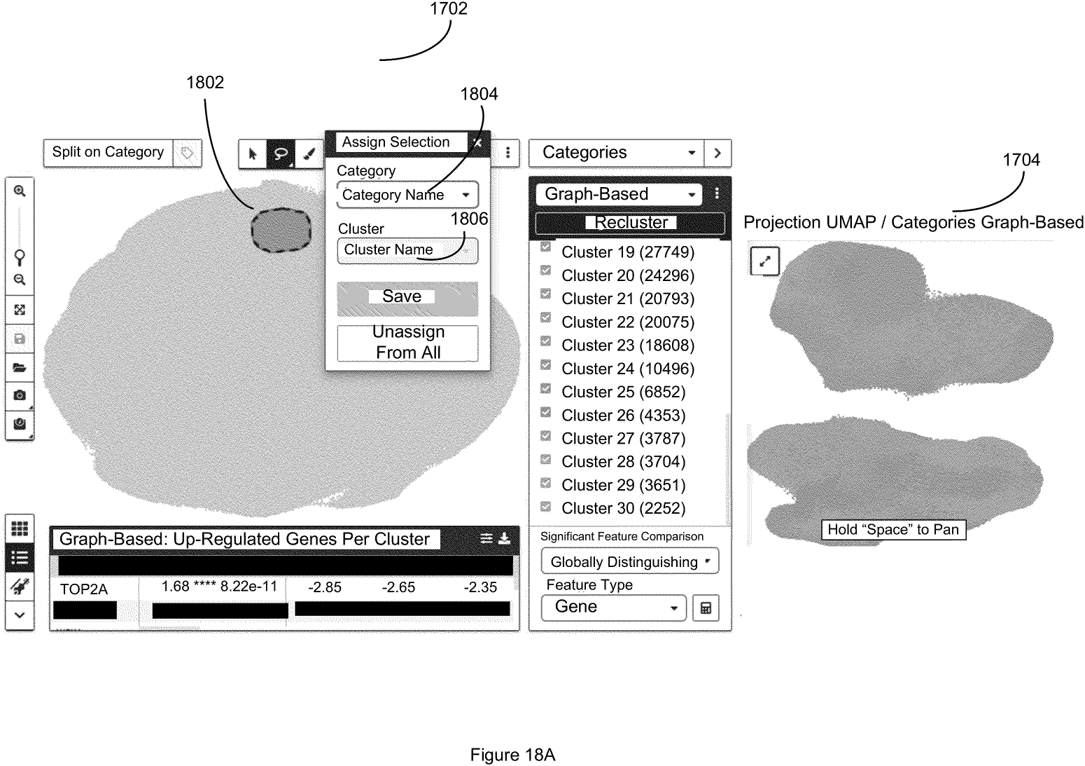

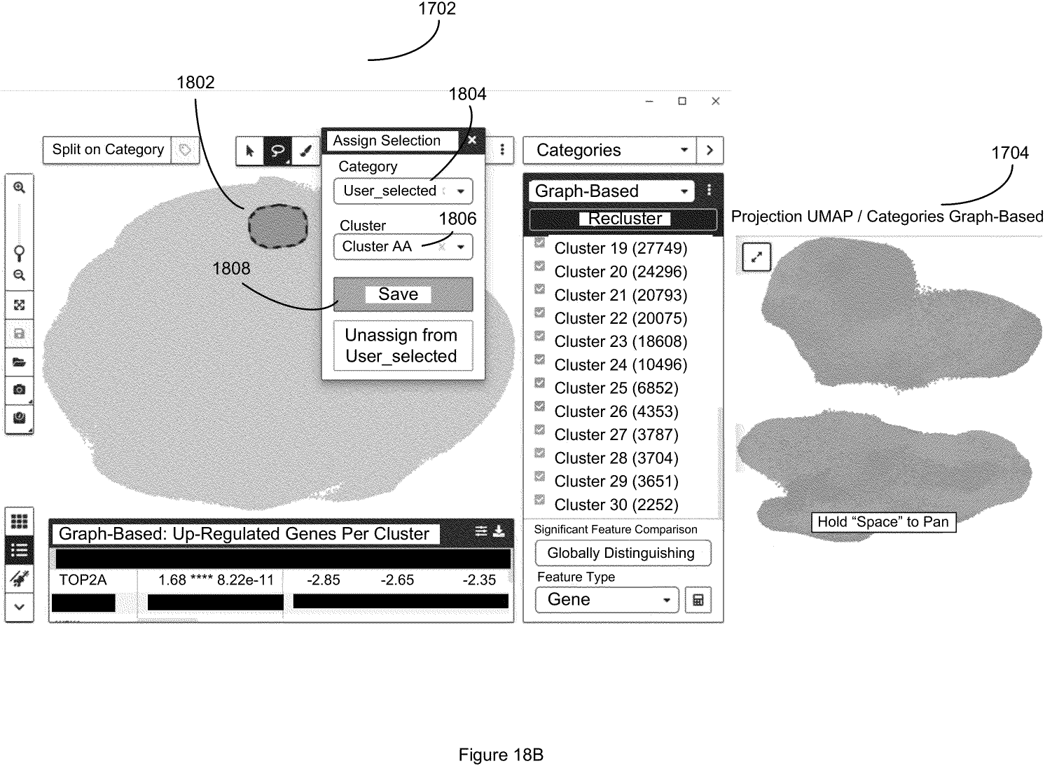

In some embodiments, the user provided category is selected from a prepopulated list of categories. In some embodiments, the user provided category is entered by the user (e.g., into a text entry affordance). For example, as illustrated in FIGS. 18A-B, a selected subset of the two-dimensional spatial arrangement of the plurality of entities 1802 is assigned to a user selected category 1804 and/or a user selected cluster 1806. In some embodiments, the visualization system further includes a user affordance 1808 for saving a selected subset to the respective category and/or cluster.

Referring to Block 250, the method further comprises modifying the discrete attribute value dataset to store an association of each respective entity in the plurality of entities to the user provided category.

For example, turning to FIG. 4, by selecting affordance 450, a dropdown menu (not shown) is provided that shows all the different categories 170 that are associated with the discrete attribute value dataset 120. In some embodiments, where there is a category 170 in a discrete attribute value dataset 120 having classes 172, each respective entity in the discrete attribute value dataset 120 is a member of each respective category 170 and one of the classes 172 of each respective category 170. In some such embodiments, where the dataset comprises a plurality of categories 170, each respective entity in the discrete attribute value dataset 120 is a member of each respective category 170, and a single class of each respective category 170.

In some embodiments, where there is a category 170 in a discrete attribute value dataset 120 that has no underlying classes 172, a subset of the entities in the dataset 120 are a member of the category 170.

In some embodiments, where there is a category 170 in a discrete attribute value dataset 120 having subclasses 172, only a portion of the respective entities in the dataset 120 are a member of the category 170. Moreover, each entity in the portion of the respective entities is independently in any one of the respective classes 172 of the category 170.

As illustrated in FIG. 4, a user can select or deselect any category 170. As further illustrated, a user can select or deselect any combination of subcategories 172 in a selected category 170. Referring to FIG. 4, in some embodiments, the user is able to click on a single cluster 158 (the clusters 1-11 are labeled as 172-1-2, 172-1-3, 172-1-4, 172-1-5, 172-1-6, 172-1-7, 172-1-8, 172-1-9, 172-1-10, and 172-1-11 respectively, in FIG. 4) to highlight it in the plot 420. In some embodiments, when the user clicks on a highlighted cluster 158 in the plot 420, the highlighting is removed from the selected cluster.

The presentation of the data in the manner depicted in FIG. 4 advantageously provides the ability to determine the reference sequences 122 whose discrete attribute values 124 separates (discriminates) classes 172 within a selected category based upon their discrete attribute values. To further assist with this, the significant reference sequences (e.g., Sig. genes) affordance 450 is selected thereby providing two options, a globally distinguishing option 452 and a locally distinguishing option (not shown in FIG. 4).

Referring to FIG. 4, the globally distinguishing option 452 identifies the reference sequences 122 whose discrete attribute values 124 within the selected classes 172 statistically discriminate with respect to the entire discrete attribute value dataset 120 (e.g., finds genes expressed highly within the selected clusters 172, relative to all the clusters 172 in the dataset 120). The locally distinguishing option identifies the reference sequences whose discrete attribute values discriminate the selected clusters (e.g., class 172-1-1 and class 172-1-11 in FIG. 4) without considering the discrete attribute values 124 in classes 72 of entities that have not been selected (e.g., without considering classes 172-1-2 through 172-1-10 of FIG. 4).

Advantageously, with reference to FIG. 5, the systems and methods of the present disclosure allow for the creation of new categories 170 using the upper panel 420 and any number of classes 172 within such categories using lasso 552 or draw selection tool 553 of FIG. 4. Thus, user identification of entity subtypes (classes 172) can be done by selecting a number of entities displayed in the upper panel 420 with the lasso tools. Moreover, they can also be selected from the lower panel 404 (e.g., the user can select a number of entities by their discrete attribute values). In this way, a user can drag and create a class 172 within a category 170. The user is prompted to name the new category 170 and the new class (cluster) 172 within the category. The user can create multiple classes of entities within a category. For instance, the user can select some entities using affordance 552 or 553, assign them to a new category (and to a first new class within the new category). Then the user selects additional entities using tools 552 or 553 and, once selected, assigns the newly selected entities to the same new category 170, but now to a different new class 172 in the category. Once the classes 172 of a category have been defined in this way, the user can compute the reference sequences whose discrete attribute values 124 discriminate between the identified user defined classes. In some such embodiments, such operations proceed faster than with categories that make use of all the entities in the discrete attribute value dataset 120 because fewer numbers of entities are involved in the computation. In some embodiments, the speed of the algorithm to identify reference sequences that discriminate classes 172 is proportional to the number of classes 172 in the category 170 times the number of entities that are in the analysis.

As illustrated in FIG. 4, the differential value 162 for each reference sequence 122 in the plurality of entities for each cluster 158 is illustrated in a color-coded way to represent the log₂ fold change in accordance with color key 408. In accordance with color key 408, those reference sequences 122 that are upregulated in the entities of a particular cluster 158 relative to all other clusters are assigned more positive values, whereas those reference sequences 122 that are down-regulated in the entities of a particular cluster 158 relative to all other clusters are assigned more negative values. In some embodiments, the heat map can be exported to persistent storage (e.g., as a PNG graphic, JPG graphic, or other file formats).

Referring to FIG. 4, advantageously, affordance 450 can be used to toggle to other visual modes. In FIG. 4, a particular “Categories” mode, “Graph based” (170) is depicted, which refers to the use of a Louvain modularity algorithm to cluster discrete attribute value 124. However, by selecting affordance 450, other options are displayed for affordance 170. In particular, in addition to the “Categories” option that was displayed in FIG. 4, “Gene Expression” can be selected as options for affordance 450.

### Graphics Processing

In some embodiments, the displaying the two-dimensional spatial arrangement of the plurality of entities on the display comprises submitting one or more discrete attribute values to a graphics processing unit (e.g., a graphics card).

Accordingly, another aspect of the present disclosure provides a visualization system comprising a main processor, a graphics processing unit, a memory, and a display, the memory storing instructions for using the main processor to perform a method for evaluating one or more biological samples. The method comprises obtaining a discrete attribute value dataset derived by nucleic acid sequencing (e.g., single cell or single nuclei sequencing) of the one or more biological samples, where the discrete attribute value dataset comprises a corresponding discrete attribute value for each reference sequence in a plurality of reference sequences for each respective entity in a plurality of entities (e.g., at least 100,000 entities) in the one or more biological samples.

The method further includes displaying the plurality of entities on the display in a two-dimensional spatial arrangement in which each respective entity in the plurality of entities is independently assigned a unique two-dimensional position. A user selection of a subset of the two-dimensional spatial arrangement on the display is received, and, responsive to the user selection, a data structure that comprises the unique two-dimensional position of each entity in the subset of entities in the two-dimensional spatial arrangement is created. The data structure is submitted to the graphics processing unit with a uniform, thereby recoloring the subset of entities on the display in accordance with the uniform.

In some embodiments, the foregoing aspect comprises any one or more of the embodiments disclosed herein, including biological samples, discrete attribute value datasets, entities, spatial arrangements, visualization, and/or subset selection, or any substitutions, modifications, additions, deletions, and/or combinations thereof, as will be apparent to one skilled in the art.

In some embodiments, each respective entity in the plurality of entities is displayed as a point in the two-dimensional spatial arrangement, each respective point in a plurality of points having a unique two-dimensional position in the two-dimensional spatial arrangement. Thus, in some embodiments, the display, visualization, and/or selection of one or more points refers to the display, visualization, and/or selection of a corresponding one or more entities.

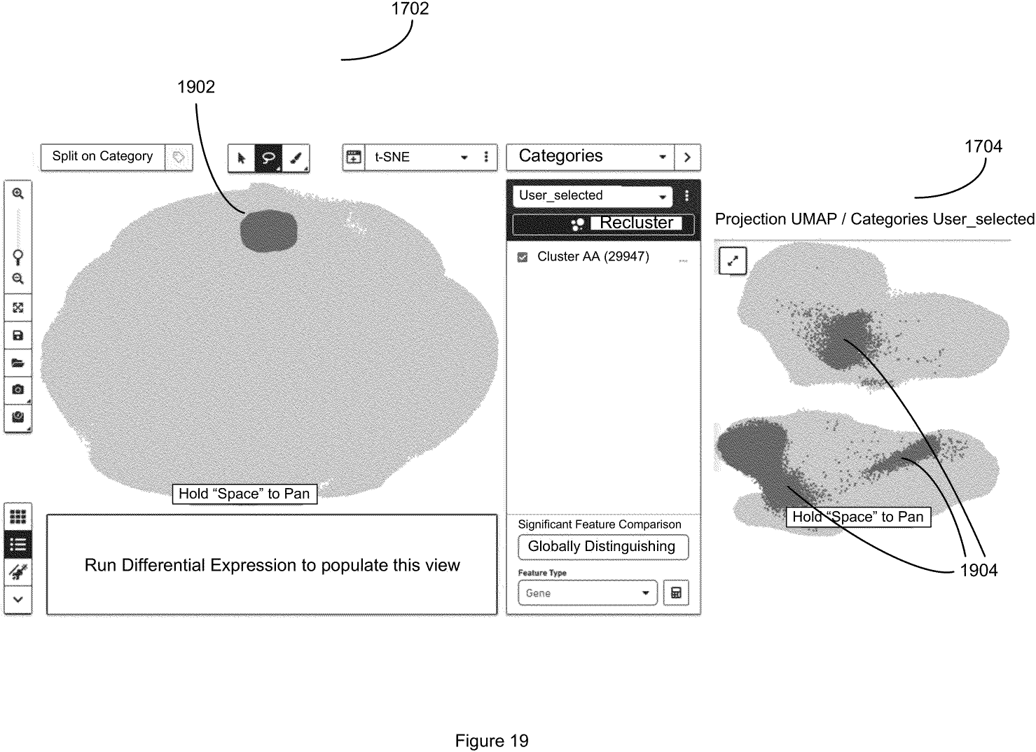

Accordingly, in some embodiments, a user selection of a subset of the two-dimensional spatial arrangement on the display is displayed such that the selected portion can be distinguished from the non-selected portion on the display. In some such embodiments, the selected portion is indicated by a graphical indicator (e.g., a change in color, a change in shading, a change in pattern, and/or a change in texture) relative to the non-selected portion. For instance, as illustrated in FIGS. 18A-B, initial selection of a subset 1802 of the two-dimensional spatial arrangement is distinguishable from the non-selected portion by a dashed line indicating the perimeter of the selected portion and a first change in color indicating the area of the selected portion. All other non-selected portions of the two-dimensional spatial arrangement are presented in untextured grayscale. Furthermore, as illustrated in FIG. 19, selection and assignment of a subset to a user provided category renders the selected subset 1902 distinguishable by a second change in color, other than the first change in color for initial selection 1802, indicating the area of the selected portion.

In some embodiments, the uniform is a constant value that indicates a color. For instance, in some embodiments, the uniform is an RGB value, a YCbCr value, a YUV value, an HSV value, an HSL value, an LCh value, a CMYK value and/or a CMY value.

In some embodiments, the display further displays one or more brush tools for use in user selection of the subset of the two-dimensional spatial arrangement on the display. In some embodiments, the one or more brush tools are customizable. For instance, in some embodiments, the one or more brush tools can be adjusted for brush thickness, brush shape, brush (e.g., selection indicator) color, or texture (e.g., pencil, pen, paintbrush, highlighter etc.). In some embodiments, the selection is performed using a lasso tool rather than a brush tool. In some embodiments, the display further displays one or more eraser tools for use in removing one or more selected points from the subset of the two-dimensional spatial arrangement on the display. In some embodiments, the one or more eraser tools are customizable. For instance, in some embodiments, the one or more eraser tools can be adjusted for thickness, shape, and/or toggle capability (e.g., remove all selected points from a subset using a single click).

As described above, in some embodiments, the display of the selected subset of the two-dimensional spatial arrangement comprises creating a data structure that comprises the unique two-dimensional position of each point (e.g., entity) in the subset of selected points (e.g., entities) in the two-dimensional spatial arrangement, and submitting the data structure to the graphics processing unit with a uniform that denotes the change in color to be displayed. In some embodiments, the data structure is a buffer.

In some embodiments, the data structure is created in real-time with user selection of the subset of the two-dimensional spatial arrangement (e.g., each selected point, entity, nucleus, and/or probe spot is added to the data structure as it is passed over by the brush tool). In some embodiments, the data structure is created after user selection of the subset of the two-dimensional spatial arrangement is completed (e.g., all selected points, entities, nuclei, and/or probe spots are added to the data structure at the end of a brush stroke and/or after selection by a lasso tool is complete).

In some embodiments, one or more points (e.g., entities) are added non-contiguously to the data structure (e.g., selection of subsets can be performed at multiple times rather than all at once, such as when selecting separate non-contiguous regions of the two-dimensional spatial arrangement).

Advantageously, by storing the two-dimensional positions of each selected data point (e.g., entity) in the two-dimensional spatial arrangement, the display of the selected subset including such visual modifications as color, texture, and/or line changes and/or class or category assignments will result in processing of only the selected data points that are submitted to the graphics processing unit, rather than of all contiguous data points between selected points, or of all data points in the discrete attribute value dataset.

For instance, instead of displaying a modification for each respective entity in the plurality of entities (e.g., recoloring), only the selected entities are stored in the data structure (e.g., buffer). In some implementations, selection of a subset of entities selects less than 30%, less than 20%, less than 10%, less than 5%, less than 1%, less than 0.5%, less than 0.1%, less than 0.05%, or less than 0.01% of the total entities in the plurality of entities. Thus, storage of the selected entities in the data structure for submission to the graphics processing unit and subsequent modification and display is advantageous in that it can reduce the volume of data points for processing by an equivalent factor (e.g., at least 10X, at least 20X, at least 100X, at least 200X, at least 1000X, at least 2000X, or at least 10,000X). Accordingly, storage of selected entities in the data structure can reduce the computational burden of processing and displaying data point selection and enhance the speed and efficiency with which user interaction on the visualization system is performed.

In some embodiments, storage of the unique two-dimensional position of each entity in the subset of entities in the two-dimensional spatial arrangement in the data structure is performed regardless of whether the entities in the subset of entities are stored contiguously in the discrete attribute value dataset (e.g., the original buffer). Such an implementation is further advantageous in that it improves upon systems that rely on linear contiguity of data for updating existing buffers (e.g., in order to update only a first point and a second point, the two points must follow one another in memory 111.

### Visualization of Multiple Windows

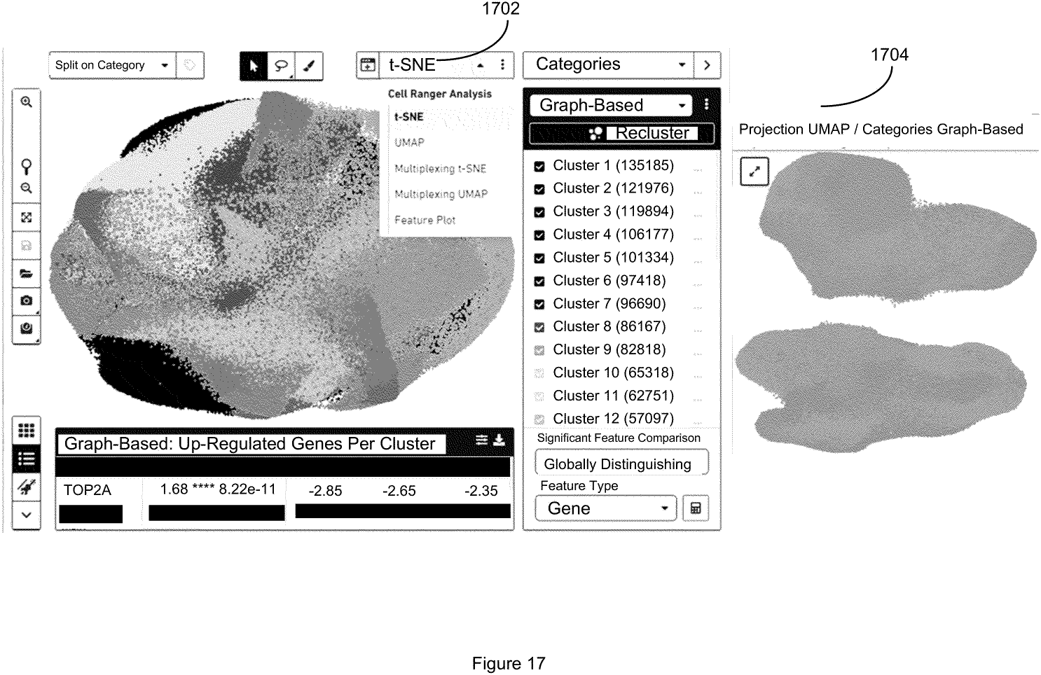

In some embodiments, a plurality of spatial projections (e.g., images and/or graphical representations) for a respective two-dimensional spatial arrangement of a respective biological sample can be concurrently viewed. Similarly, in some embodiments, corresponding spatial projections (e.g., images and/or graphical representations) for a plurality of biological samples can be concurrently viewed. In some such embodiments, the user will arrange such viewers side by side so that comparisons between the images of respective spatial projections, regions of interest 121, and/or biological samples can be made. Such aggregated datasets will have overarching clusters that span multiple spatial arrangements, as well as t-SNE and UMAP projections. For instance, FIG. 17 illustrates concurrent visualization of a plurality of spatial projections for a respective two-dimensional spatial arrangement of a respective biological sample, where the plurality of spatial projections includes a first spatial projection 1702 representing a t-SNE projection and a second spatial projection 1704 representing a UMAP projection. In FIG. 17, clusters are indicated in both spatial projection 1702 and spatial projection 1704 by colored indicia for each respective entity in the plurality of entities that belongs to the respective cluster.

To allow users to see common characteristics or compare different spatial projections and/or spatial arrangements at once, one aspect of the present disclosure makes use of novel linked windows. Referring to FIG. 13A, clicking on the “Add Window” affordance 1302 brings up a list of projections 1305 (see FIG. 13B) for the discrete attribute value dataset to open in a linked window. Thus, referring to FIG. 13B, the projection SR-Custom-22 is visible in panel 1304 and the user has the option of adding a window for projections t-SNE 1305-1, SR-Custom-24 1305-3, UMAP 1305-4, feature plot 1305-5 or, in fact, another instance of SR-Custom-22 1305-1. Clicking on one of these projections opens that projection in a smaller window within the operating system. For instance, clicking on SR-Custom-24 1305-3 in FIG. 13B causes a smaller window 1306 with this projection to be concurrently displayed with SR-Custom-22 1305-2 as illustrated in FIG. 13C. In FIG. 13C, it is clear from menu 1308 that the projection 1310 to the far left in the panel is that of SR-Custom-22 1305-2. One can create multiple linked windows for a single dataset in this manner as illustrated in FIG. 13D. In FIG. 13D, the main panel 1320 is that of projection t-SNE 1305-1 while smaller windows 1322 and 1324 are for projections SR-Custom-22 1305-2 and SR-Custom-24 1305-3 respectively. In some embodiments, linked windows (e.g., windows 1322 and 1324 of FIG. 13D) open initially in miniaturized view as illustrated in FIG. 13D, where only the projection and a button 1326 to expand the window to a full panel is shown. As illustrated in FIG. 13D, when using a mouse cursor to hover over a linked window (e.g., window 1322), more options 1328 and 1330 are revealed that provide a subset of common actions, such as the ability to pan and zoom a linked window. However, the linked windows are still predominantly controlled by manipulating the original, or anchor window 1320.

Referring to FIG. 13D, changes to the anchor window 1320 will propagate automatically to the other linked windows (e.g., windows 1322 and 1324), such as using toggles 1332 to change active clusters (which clusters are displayed across all the linked windows), selecting an individual cluster, creating a new cluster or modifying a cluster, selecting one or more genes to show feature expression (gene, antibody, peak), changing cluster membership, changing individual cluster colors or the active expression color scale, in (VDJ mode) selecting active clonotypes, and in (ATAC mode) selecting transcription factor motifs. However, features such as panning, zooming, spatial image settings (pre-save) such as color, brightness, contrast, saturation and opacity, selected region of interest, and window sizes remain independent in the anchor and linked windows.

Referring to FIG. 13D, it is possible to expand a linked window from mini-mode to access the full range of visualization options by clicking on the expand affordance 1326. Clicking the window again will shrink it back to mini-mode. In some embodiments, changes to the discrete attribute value dataset 120 in any window are saved through the anchor window. Thus, referring to FIG. 13D, any change to the discrete attribute value dataset 120 in windows 1322 or 1324 must be saved through window 1320 in such embodiments.

It is also possible to have other linked windows open to view other discrete attribute value datasets 120. To avoid confusion, when multiple attribute value datasets are open, the color of the button 1334 (FIG. 13D) will signify which windows are linked. For instance, referring to FIG. 13E, windows 1370 and 1372, which represent two different regions of interest 121 for a first discrete attribute dataset 120, are linked and so have a common orange border on logo 1374, while windows 1376 and 1378, which represent two different regions of interest 121 for a second discrete attribute dataset 120, are linked and so have a common black border on logo 1380. Moreover, linked windows are not limited to spatial discrete attribute value datasets 120. Most gene expression datasets have both t-SNE and UMAP projections 121 (see U.S. Pat. Application No. 16/442,800 entitled “Systems and Methods for Visualizing a Pattern in a Dataset,” filed Jun. 17, 2019) that can be linked and viewed at the same time in a similar fashion.

FIG. 13F illustrates how linked windows can advantageously lead to rapid analysis. FIG. 13F illustrates a t-SNE plot 1380 that represents the dimensionality reduction over two regions of interest 121 (SR-CUSTOM-22 1382 and SR-CUSTOM-24 1384) within a particular discrete attribute dataset 120. There is a visual cluster 1386 in the t-SNE (Outliers/C1) that the automatic clustering did not differentiate. Cluster 1386 contains a mix of probe spots assigned to different graph-based and K-means clusters. After selecting custom cluster 1386 in the anchor window (t-SNE view 1380), it is possible to see which regions it corresponds to in the two regions of interest 1382 / 1384 in the other linked windows. Zooming into each region between the two regions of interest 1382 / 1384 shows that there is common, tubular morphology under all spatial spots that are members of cluster 1386. There are also a variety of significant genes associated with these regions. In this manner, the present disclosure advantageously concurrently displays information from the gene expression-based projection (t-SNE plot 1380) to detect potentially interesting regions in the spatial context (SR-CUSTOM-22 1382 and SR-CUSTOM-24 1384). Using linked windows avoids having to jump back and forth, making the investigation fluid and intuitive.

While linked windows have been illustrated in conjunction with showing mRNA-based UMI abundance overlayed on source images, they can also be used to illustrate the spatial quantification of other analytes, either superimposed on images of their source tissue or arranged in two-dimensional space using dimension reduction algorithms such as t-SNE or UMAP, including cell surface features (e.g., using the labelling agents described herein), mRNA and intracellular proteins (e.g., transcription factors), mRNA and cell methylation status, mRNA and accessible chromatin (e.g., ATAC-seq, DNase-seq, and/or MNase-seq), mRNA and metabolites (e.g., using the labelling agents described herein), a barcoded labelling agent (e.g., the oligonucleotide tagged antibodies described herein) and a V(D)J sequence of an immune cell receptor (e.g., T-cell receptor), mRNA and a perturbation agent (e.g., a CRISPR crRNA/sgRNA, TALEN, zinc finger nuclease, and/or antisense oligonucleotide as described herein). For general disclosure on how such analytes are spatially quantified, see, U.S. Patent Publication No. US 2021-0155982, entitled “Pipeline for Analysis of Analytes”, which is hereby incorporated by reference. For general disclosure on how ATAC is spatially quantified using, for example clustering and/or t-SNE (where such cluster and/or t-SNE plots can be displayed in linked windows), see, U.S. Publication No. US-2020105373-A1 entitled “Systems and Methods for Cellular Analysis Using Nucleic Acid Sequencing” which is hereby incorporated by reference. For general disclosure on how V(D)J sequences are spatially quantified using, for example clustering and/or t-SNE (where such cluster and/or t-SNE plots can be displayed in linked windows), see, U.S. Pat. Publication No. US 2018-0371545, entitled “Systems and Methods for Clonotype Screening”, which is hereby incorporated by reference.

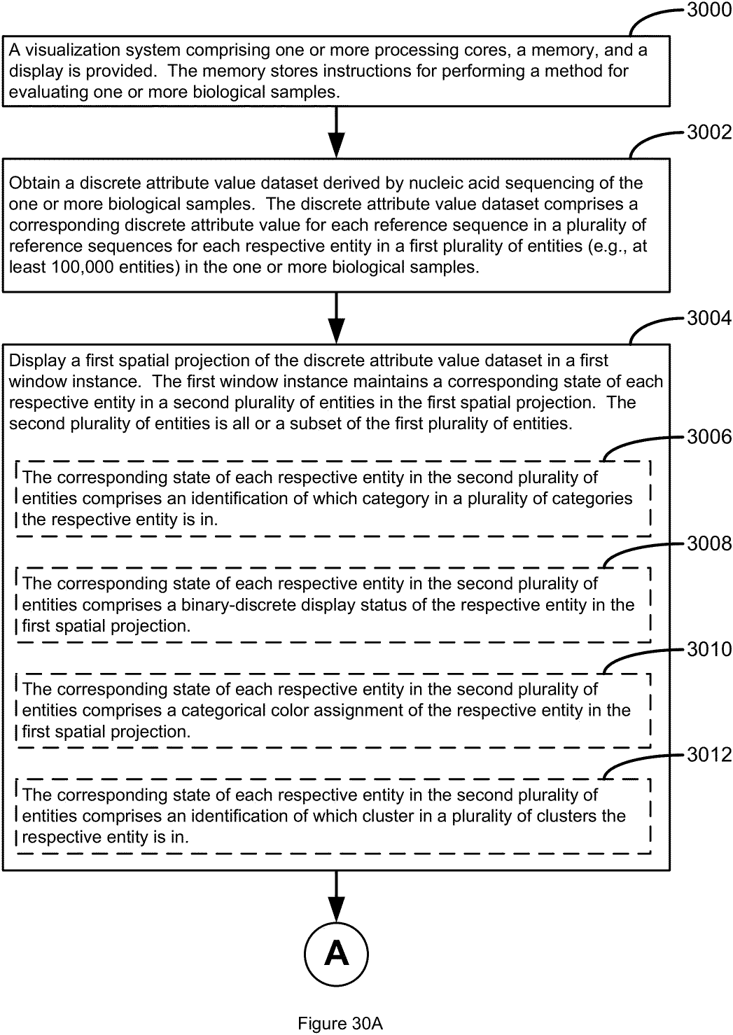

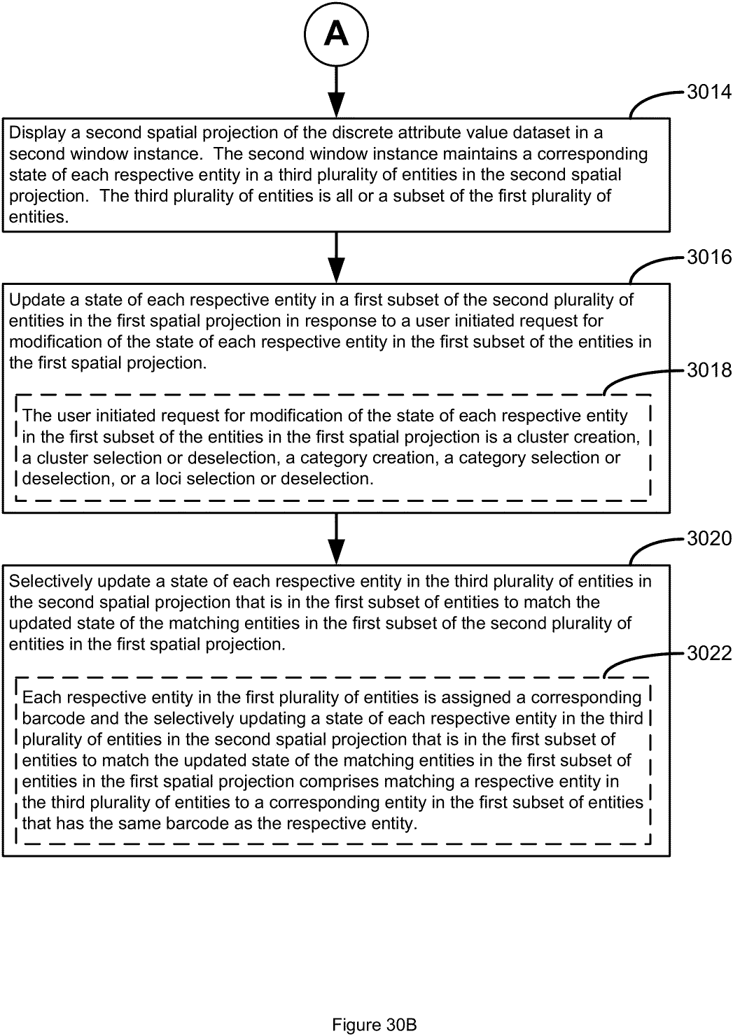

Accordingly, referring to Block 3000 of FIGS. 30A-B, the present disclosure provides a visualization system comprising one or more processing cores, a memory, and a display, the memory storing instructions for performing a method for evaluating one or more biological samples. Referring to Block 3002, the method comprises obtaining a discrete attribute value dataset derived by nucleic acid sequencing (e.g., single cell or single nuclei sequencing) of the one or more biological samples, where the discrete attribute value dataset comprises a corresponding discrete attribute value for each reference sequence in a plurality of reference sequences for each respective entity in a first plurality of entities (e.g., at least 100,000 entities) in the one or more biological samples.

Referring to Block 3004, the method includes displaying a first spatial projection of the discrete attribute value dataset in a first window instance, wherein the first window instance maintains a corresponding state of each respective entity in a second plurality of entities in the first spatial projection, where the second plurality of entities is all or a subset of the first plurality of entities.

In some embodiments, a respective state is any feature, annotation, selection status, condition, label, analytical outcome, and/or component of a respective entity. For instance, referring to Block 3006, 3008, 3010, and 3012, in some embodiments, the corresponding state of each respective entity in the second plurality of entities comprises an identification of which category in a plurality of categories the respective entity is in. In some embodiments, the corresponding state of each respective entity in the second plurality of entities comprises a binary-discrete display status of the respective entity in the first spatial projection. In some embodiments, the corresponding state of each respective entity in the second plurality of entities comprises a categorical color assignment of the respective entity in the first spatial projection. In some embodiments, the corresponding state of each respective entity in the second plurality of entities comprises an identification of which cluster in a plurality of clusters the respective entity is in.

Referring to Block 3014, the method further includes displaying a second spatial projection of the discrete attribute value dataset in a second window instance, where the second window instance maintains a corresponding state of each respective entity in a third plurality of entities in the second spatial projection, where the third plurality of entities is all or a subset of the first plurality of entities.

The method further comprises, referring to Block 3016, updating a state of each respective entity in a first subset of the second plurality of entities in the first spatial projection in response to a user initiated request for modification of the state of each respective entity in the first subset of the entities in the first spatial projection. For instance, as described in Block 3018, in some embodiments, the user initiated request for modification of the state of each respective entity in the first subset of the entities in the first spatial projection is a cluster creation, a cluster selection or deselection, a category creation, a category selection or deselection, or a loci selection or deselection.

Referring to Block 3020, the method includes selectively updating a state of each respective entity in the third plurality of entities in the second spatial projection that is in the first subset of entities to match the updated state of the matching entities in the first subset of the second plurality of entities in the first spatial projection.

Accordingly, the method comprises linking a first state for each respective entity in the first spatial projection with a corresponding state for the respective entity in the second spatial projection, between the first window and the second window. For instance, where the user initiated request for modification of the state of each respective entity in the first subset of the entities in the first spatial projection is a cluster creation, a cluster selection or deselection, a category creation, a category selection or deselection, or a loci selection or deselection, the method comprises linking cluster selection, cluster creation, loci selection, cluster membership, or cluster indicia selection between the first window and the second window. An example of window linking is illustrated in FIGS. 18A-B and 19. FIG. 18A illustrates concurrent visualization of a plurality of spatial projections for a respective two-dimensional spatial arrangement of a respective biological sample, where the plurality of spatial projections includes a first spatial projection 1702 representing a t-SNE projection and a second spatial projection 1704 representing a UMAP projection. FIG. 18B illustrates selection of a subset of the two-dimensional spatial arrangement of the plurality of entities 1802 and subsequent assignment of the selected subset to a user selected category 1804 and/or a cluster 1806 via a user affordance 1808. FIG. 19 illustrates the concurrent visualization of, in the first spatial projection 1702, the created cluster 1902, and, in the second spatial projection 1704, the corresponding state (e.g., linked clusters) 1904 of each respective entity that is in the created cluster. Thus, FIG. 19 illustrates the use of linked windows to simultaneously visualize the plurality of entities using multiple graphical representations.

Referring to Block 3022, in some embodiments, each respective entity in the first plurality of entities is assigned a corresponding barcode and the selectively updating a state of each respective entity in the third plurality of entities in the second spatial projection that is in the first subset of entities to match the updated state of the matching entities in the first subset of entities in the first spatial projection comprises matching a respective entity in the third plurality of entities to a corresponding entity in the first subset of entities that has the same barcode as the respective entity.

In some embodiments, the method can be performed for a plurality of linked windows. In some embodiments, the method is performed simultaneously for each respective window in a plurality of linked windows. In some embodiments, the method is performed for two linked windows in a plurality of windows, where the linked windows are selected by a user.

Other features and characteristics for linking states between windows are possible, as will be apparent to one skilled in the art. In some embodiments, the foregoing aspect comprises any one or more of the embodiments disclosed herein, including biological samples, discrete attribute value datasets, entities, spatial projections, visualization, and/or subset selection, or any substitutions, modifications, additions, deletions, and/or combinations thereof, as will be apparent to one skilled in the art.

### Reclustering

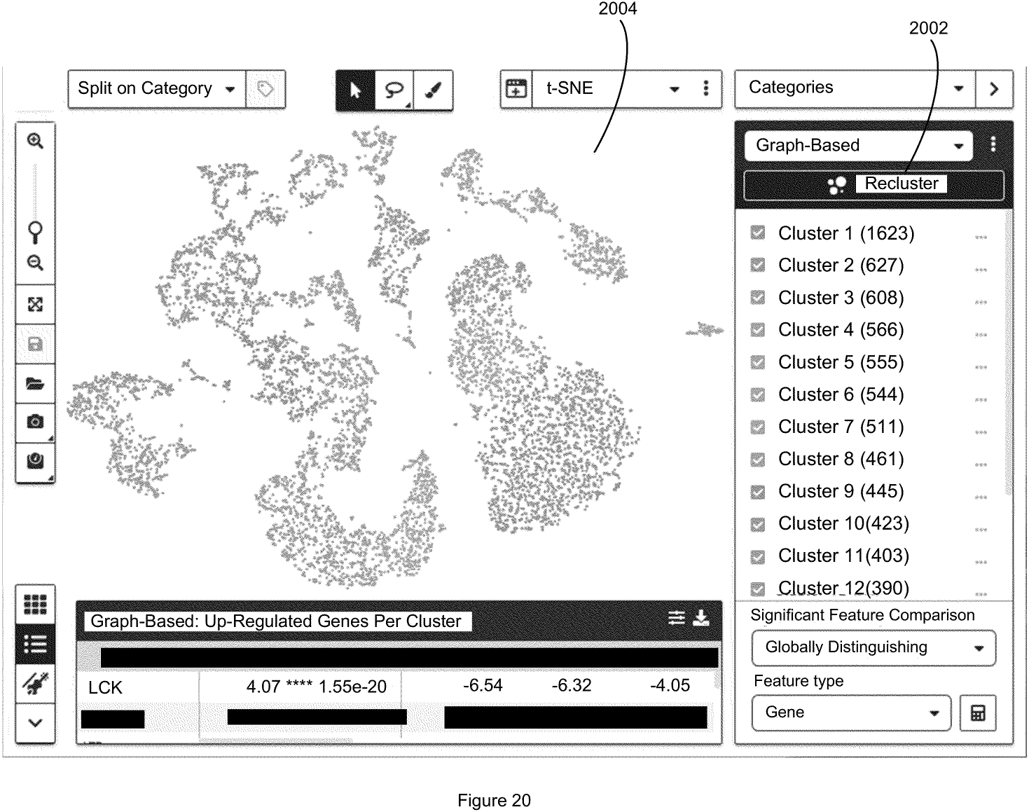

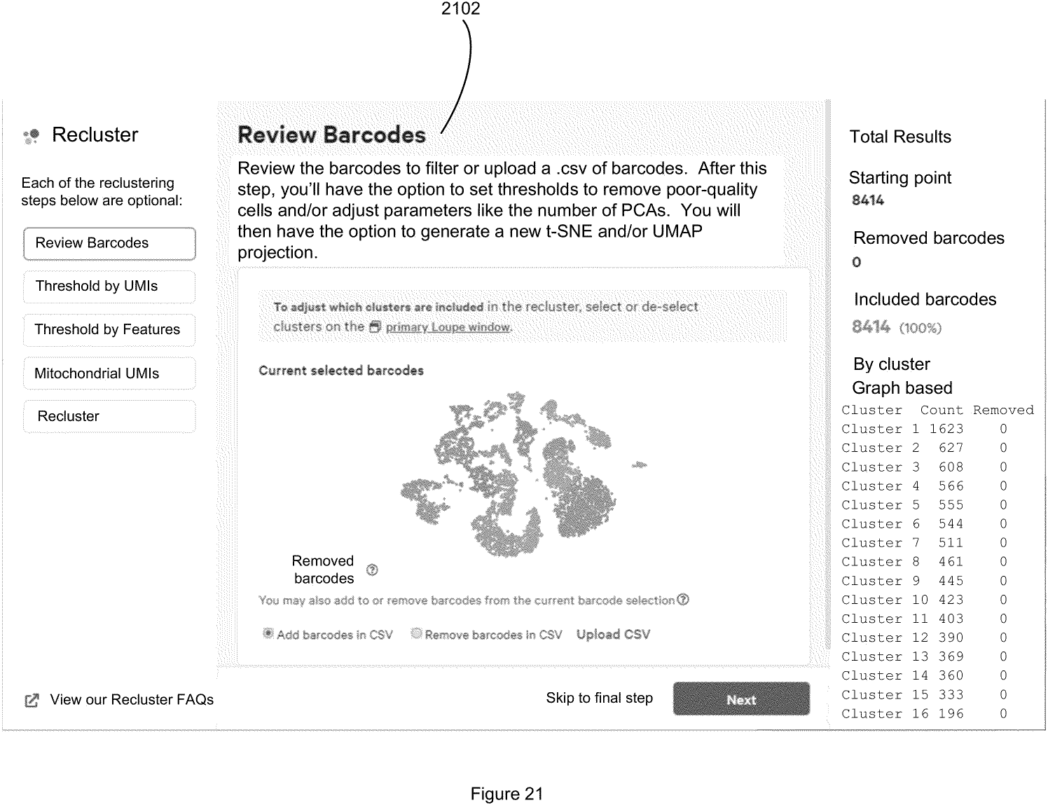

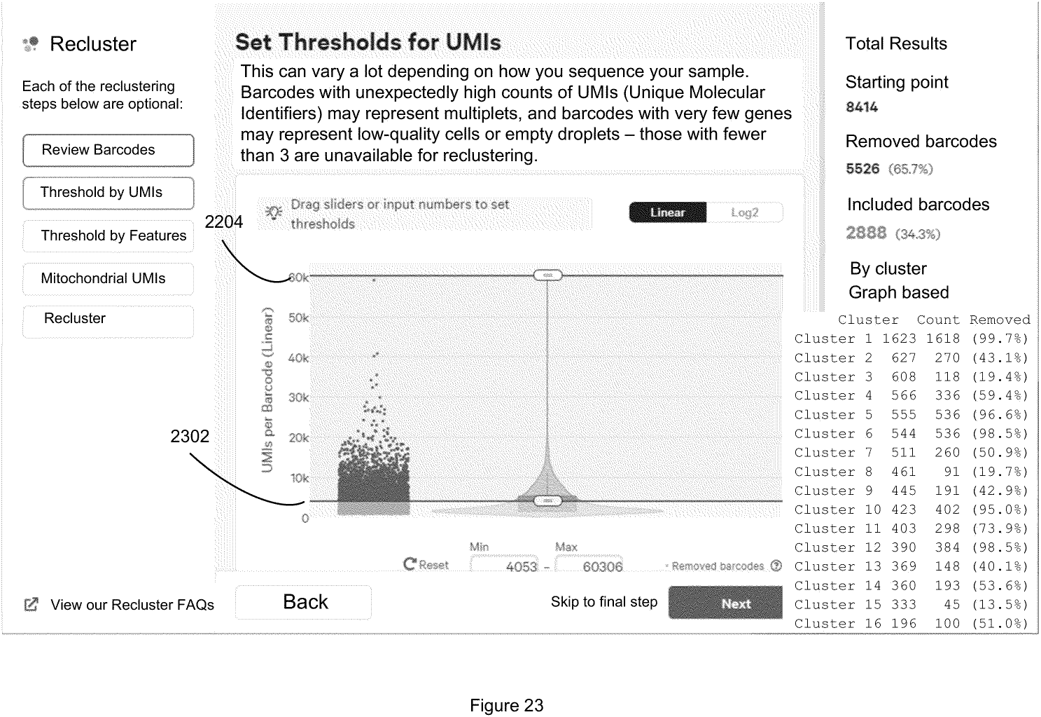

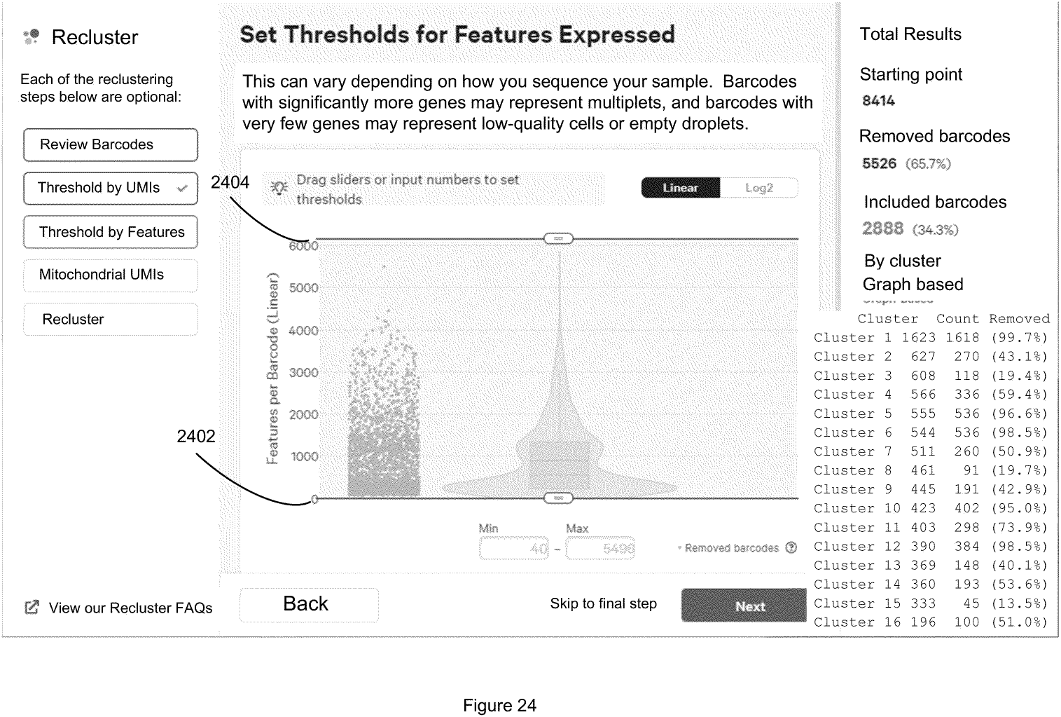

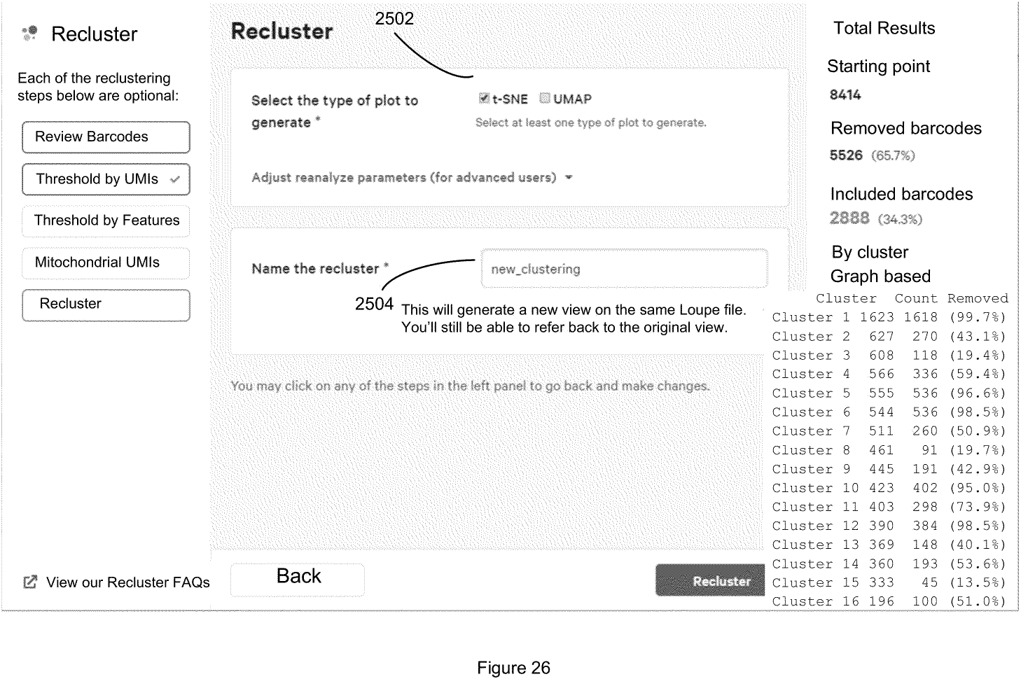

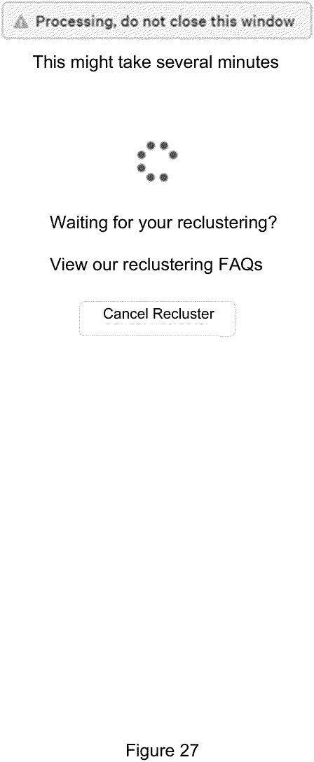

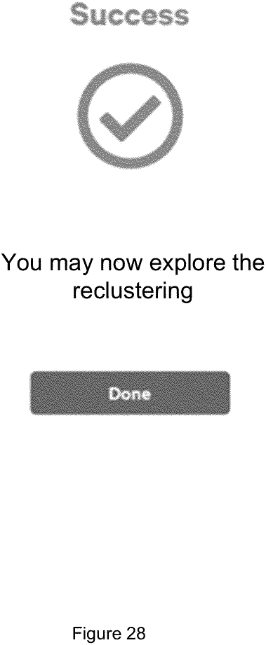

In some embodiments, the method comprises performing a second clustering after a first clustering (e.g., reclustering). FIGS. 20-29 illustrate an example visualization system for performing a process of reclustering a discrete attribute value dataset for a plurality of entities and displaying the plurality of entities in a two-dimensional spatial arrangement based on the reclustering, in accordance with some embodiments of the present disclosure.

FIG. 20 illustrates a plurality of clusters, obtained from a first clustering 2004 for a discrete attribute value dataset of a biological sample. Selection of a user affordance 2002 within the visualization system allows the user to being a reclustering process. During reclustering, the first clustering 2004 can be optionally modified by reviewing the plurality of barcodes 2102 associated with each respective entity in the plurality of entities. Following barcode review, the plurality of barcodes can be filtered, or a new plurality of barcodes can be uploaded. Additionally, clusters for reclustering can be selected or deselected by user interaction (e.g., user selection and/or deselection of target clusters).

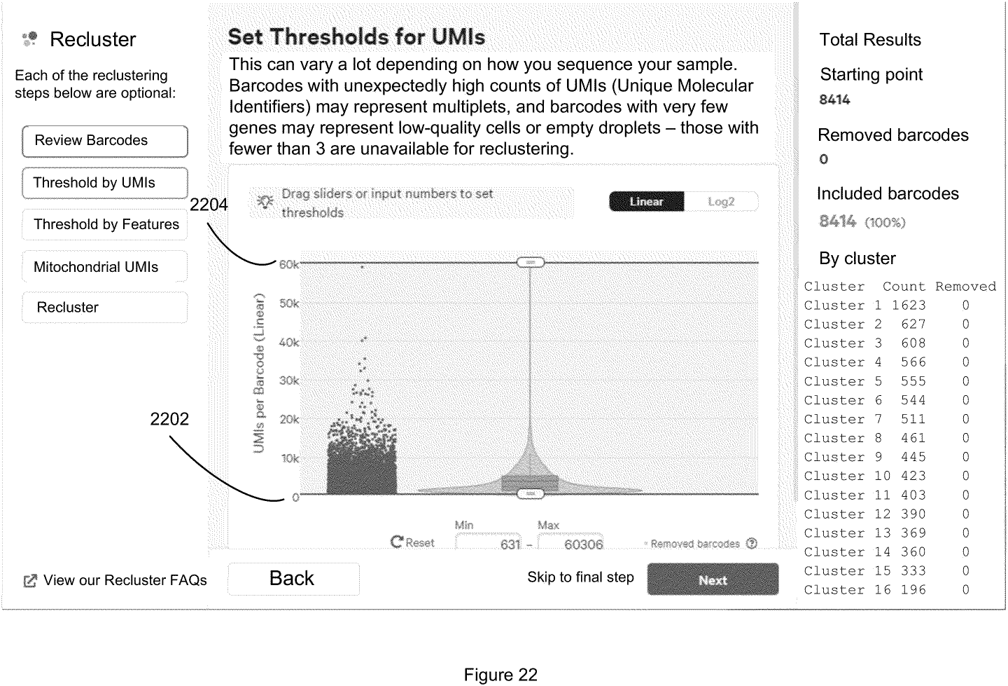

As illustrated in FIGS. 22-24, the reclustering process further comprises optionally setting thresholds to remove poor-quality entities (e.g., cells) and/or adjusting parameters (e.g., number of dimension reduction components) for analysis. For instance, a first threshold 2202 for filtering a number of unique molecular identifiers (UMI) per barcode can be adjusted to a second threshold 2302. In some embodiments, a lower threshold 2202 and/or an upper threshold 2204 can be adjusted. Setting thresholds for UMIs can improve clustering analysis by reducing the number of uninformative data points. For instance, barcodes with unexpectedly high counts of UMIs may represent multiplets of entities, while barcodes with very few UMIs may represent low-quality or empty data points (e.g., entities). In some embodiments, barcodes with fewer than 3 UMIs are excluded from reclustering analysis. Similarly, a first threshold for filtering a number of features (e.g., genes) per barcode can be adjusted to a second threshold. In some embodiments, a lower threshold 2402 and/or an upper threshold 2404 can be adjusted. Setting thresholds for features can improve clustering analysis by reducing the number of uninformative data points. For instance, barcodes with unexpectedly high counts of features (e.g., genes) may represent multiplets of entities, while barcodes with very few features (e.g., genes) may represent low-quality or empty data points (e.g., entities).

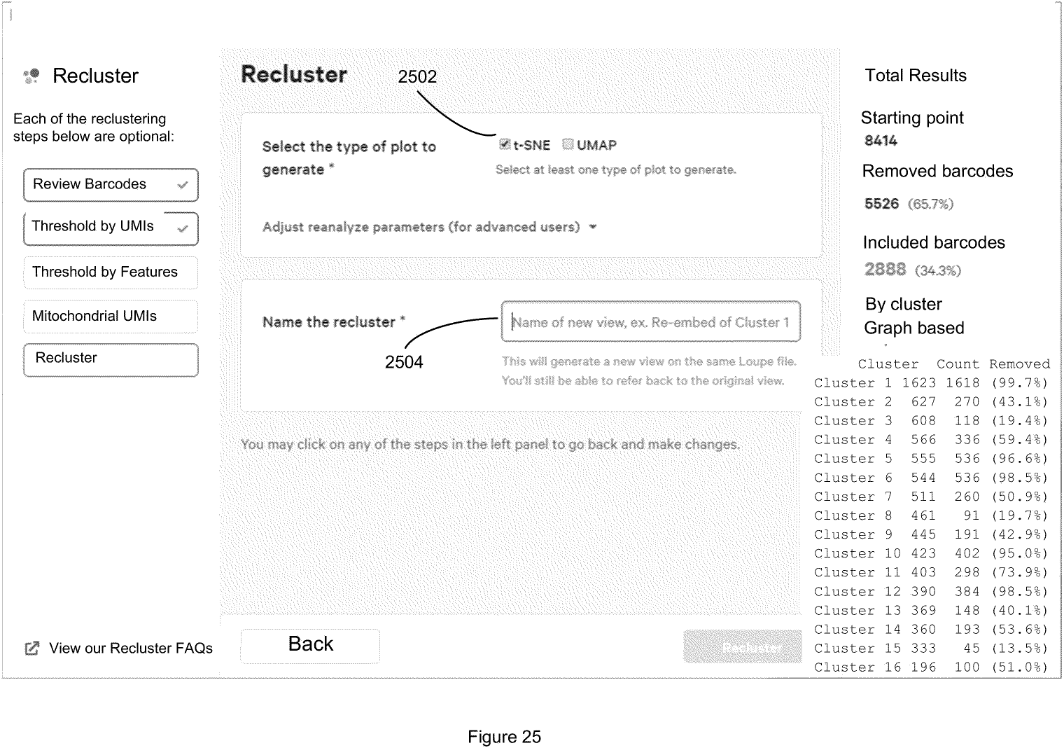

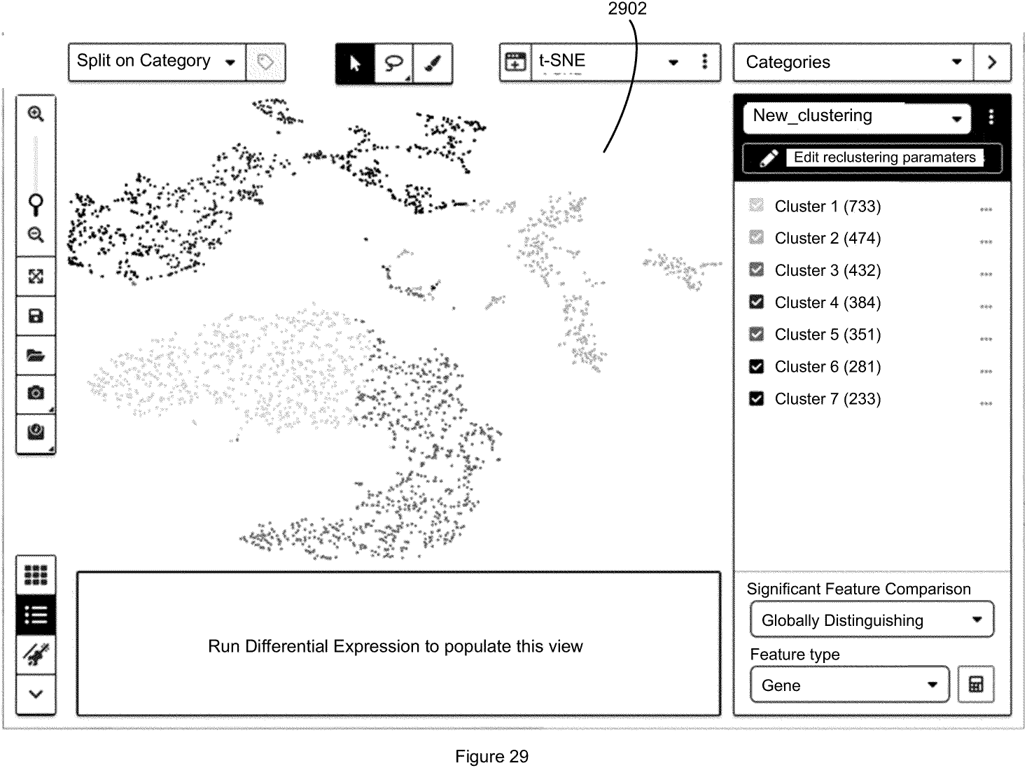

FIGS. 25, 26, 27, and 28 collectively illustrate an example visualization system for modifying a clustering of a discrete attribute value dataset for a plurality of entities using a reclustering workflow. In some embodiments, the reclustering workflow comprises optionally generating new spatial projections 2502 (e.g., t-SNE and/or UMAP projections). Additional user affordances are provided in the visualization system for naming the reclustering analysis 2504. FIG. 29 illustrates the two-dimensional spatial arrangement 2902 of a plurality of entities based on a reclustering procedure, where the generated clusters for the discrete attribute value dataset differs from the original clustering analysis 2004 illustrated in FIG. 20.

In some embodiments, the present disclosure provides a reclustering method that reduces matching (e.g., synchronization) of states between a first window (e.g., a first spatial projection) displaying an original (e.g., primary) clustering analysis and a second window (e.g., a second spatial projection) displaying a reclustering analysis. In some embodiments, the present disclosure provides a reclustering method that reduces the amount of data that is matched (e.g., synchronized) between a first window (e.g., a first spatial projection) displaying an original (e.g., primary) clustering analysis and a second window (e.g., a second spatial projection) displaying a reclustering analysis.

In some embodiments, the method comprises selectively updating each respective entity in the respective plurality of entities in a second spatial projection (e.g., a second window) that corresponds to matching selected entities in a first spatial projection (e.g., a first window) to match the updated state of the matching entities in the first spatial projection, where the updated state of the matching entities is a reclustering analysis. Accordingly, the method comprises linking a state (e.g., a reclustering analysis) for each respective entity in the first spatial projection with a corresponding state (e.g., a reclustering analysis) for the respective entity in the second spatial projection, between the first window and the second window.

In some such embodiments, the selectively updating comprises updating only the subset of entities in the plurality of entities in the second spatial projection that matches the subset of updated entities in the first spatial projection. In this way, the method advantageously reduces the number of entities to be updated to a limited subset rather than updating all of the entities in the plurality of entities in the second spatial projection.

In some embodiments, the selectively updating comprises updating the subset of entities in the plurality of entities in the second spatial projection at multiple time points throughout the reclustering process. In some embodiments, the selectively updating comprises updating the subset of entities in the plurality of entities in the second spatial projection at a single time point during the reclustering process. In some such embodiments, the selectively updating updates the subset of entities in the second spatial projection when the two-dimensional spatial arrangement of the first spatial projection is fully or nearly fully rendered. In some embodiments, the first spatial projection is rendered independently from the second spatial projection.

In some embodiments, displaying a respective spatial projection the display comprises submitting one or more discrete attribute values for a respective one or more entities to a rendering library. In some embodiments, the rendering library is Plotly. See, for example, Plotly Technologies Inc. Collaborative data science. Montreal, QC, 2015. In some embodiments, the rendering library is DeckGL (available on the Internet at deck.gl).

In some embodiments, the visualization system comprises a trace state data structure that stores one or more parameters for a respective two-dimensional spatial arrangement of the plurality of entities for the one or more biological samples. In some embodiments, the trace state data structure stores a spatial description (e.g., plot description), one or more two-dimensional positions corresponding to one or more entities (e.g., point locations), one or more color indicia, one or more opacity parameters, and/or a combination thereof.

In some embodiments, the visualization system does not include a trace state data structure. In some embodiments, the visualization system does not store trace state data including the one or more parameters for the respective two-dimensional spatial arrangement of the plurality of entities. Advantageously, this leads to a reduction in the amount of data to be stored in the visualization system, thus resulting in performance enhancements.

### Additional Visualization System Embodiments

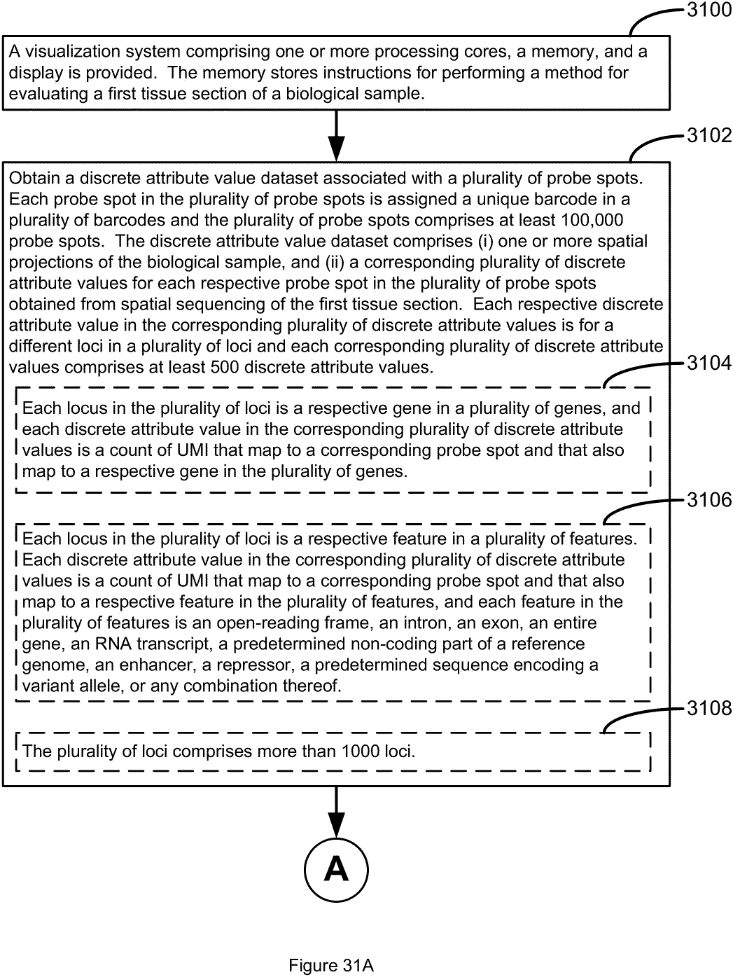

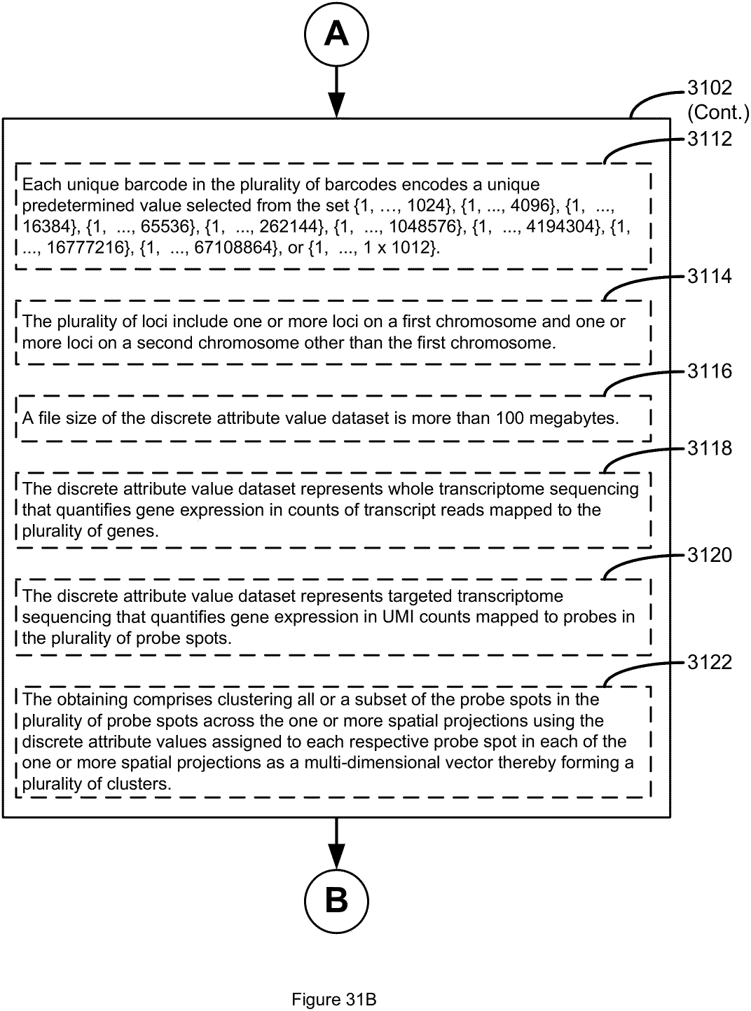

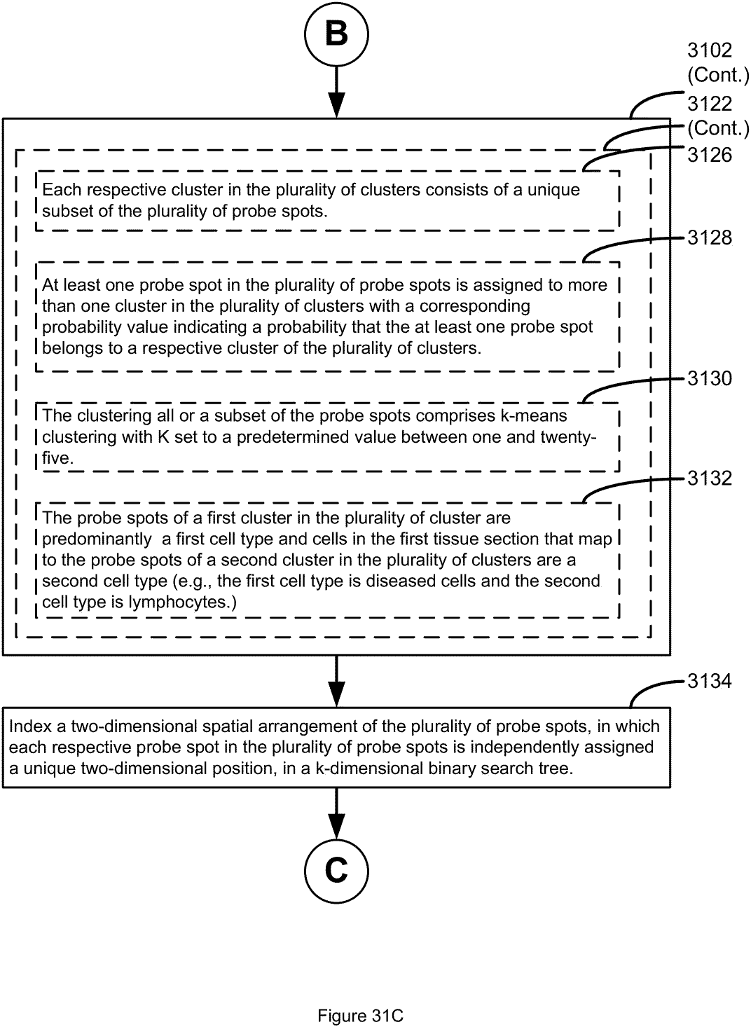

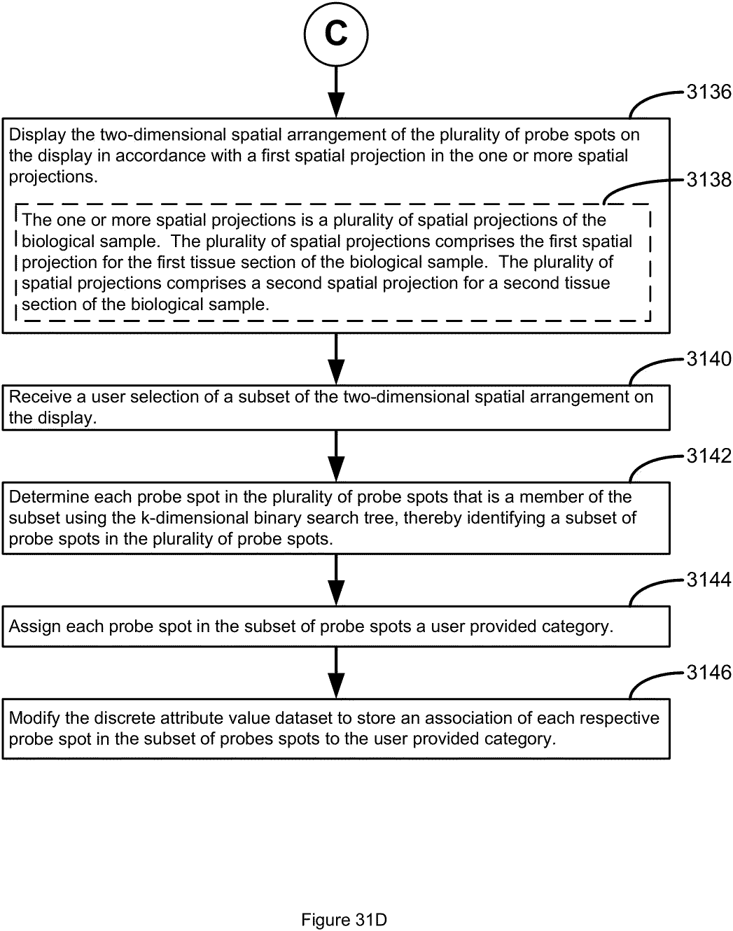

Additional embodiments for visualization systems for performing a method in accordance with the present disclosure are now detailed with reference to FIGS. 31A-D and 32A-C.

Referring to Block 3100 of FIGS. 31A-D, the present disclosure provides a visualization system comprising one or more processing cores, a memory, and a display, the memory storing instructions for performing a method for evaluating a first tissue section of a biological sample.

Referring to Block 3102, the method comprises obtaining a discrete attribute value dataset associated with a plurality of probe spots (e.g., at least 100,000 probe spots), where each probe spot in the plurality of probe spots is assigned a unique barcode in a plurality of barcodes, the discrete attribute value dataset comprising (i) one or more spatial projections of the biological sample, and (ii) a corresponding plurality of discrete attribute values (e.g., at least 500 discrete attribute values) for each respective probe spot in the plurality of probe spots obtained from spatial sequencing of the first tissue section, where each respective discrete attribute value in the corresponding plurality of discrete attribute values is for a different locus in a plurality of loci.

In some embodiments, the plurality of probe spots comprises at least 100, at least 200, at least 300, at least 400, at least 500, at least 1000, at least 2000, at least 3000, at least 4000, at least 5000, at least 10,000, at least 20,000, at least 50,000, at least 100,000, at least 200,000, at least 300,000, at least 400,000, at least 500,000, at least 600,000, at least 700,000, at least 800,000, at least 900,000, at least 1 million, or at least 2 million probe spots. In some embodiments, the plurality of probe spots comprises no more than 5 million, no more than 1 million, no more than 500,000, no more than 100,000, no more than 50,000, no more than 10,000, or no more than 1000 probe spots. In some embodiments, the plurality of probe spots comprises from 500 to 100,000, from 50,000 to 500,000, from 100,000 to 1 million, or from 500,000 to 2 million probe spots. In some embodiments, the plurality of probe spots falls within another range starting no lower than 100 probe spots and ending no higher than 5 million probe spots.

In some embodiments, the plurality of barcodes comprises at least 100, at least 200, at least 300, at least 400, at least 500, at least 1000, at least 2000, at least 3000, at least 4000, at least 5000, at least 10,000, at least 20,000, at least 50,000, at least 100,000, at least 200,000, at least 300,000, at least 400,000, at least 500,000, at least 600,000, at least 700,000, at least 800,000, at least 900,000, at least 1 million, or at least 2 million barcodes. In some embodiments, the plurality of barcodes comprises no more than 5 million, no more than 1 million, no more than 500,000, no more than 100,000, no more than 50,000, no more than 10,000, or no more than 1000 barcodes. In some embodiments, the plurality of barcodes comprises from 500 to 100,000, from 50,000 to 500,000, from 100,000 to 1 million, or from 500,000 to 2 million barcodes. In some embodiments, the plurality of barcodes falls within another range starting no lower than 100 barcodes and ending no higher than 5 million barcodes.

In some embodiments, the one or more spatial projections of the biological sample comprises at least 2, at least 3, at least 4, at least 5, at least 6, at least 7, at least 8, at least 9, at least 10, at least 15, at least 20, at least 25, at least 30, at least 35, at least 40, or at least 50 spatial projections. In some embodiments, the one or more spatial projections of the biological sample comprises no more than 100, no more than 50, no more than 40, no more than 30, no more than 20, or no more than 10 spatial projections. In some embodiments, the one or more spatial projections of the biological sample comprises from 2 to 10, from 5 to 20, from 10 to 50, or from 5 to 100 spatial projections. In some embodiments, the one or more spatial projections of the biological sample falls within another range starting no lower than 2 spatial projections and ending no higher than 100 spatial projections.

In some embodiments, each corresponding plurality of discrete attribute values comprises at least 1000, at least 2000, at least 5000, at least 10,000, at least 20,000, at least 50,000, at least 100,000, at least 200,000, at least 300,000, at least 400,000, at least 500,000, at least 600,000, at least 700,000, at least 800,000, at least 900,000, or at least 1 million discrete attribute values. In some embodiments, the discrete attribute value dataset comprises no more than 2 million, no more than 1 million, no more than 500,000, no more than 100,000, no more than 50,000, no more than 10,000, or no more than 1000 discrete attribute values. In some embodiments, the discrete attribute value dataset comprises from 10,000 to 100,000, from 50,000 to 500,000, or from 100,000 to 2 million discrete attribute values. In some embodiments, the discrete attribute value dataset falls within another range starting no lower than 1000 discrete attribute values and ending no higher than 2 million discrete attribute values.

In some embodiments, the discrete attribute value dataset, probe spots, entities, barcodes, spatial projections, loci, reference sequences, sequencing, and/or biological sample comprises any one or more of the embodiments for discrete attribute value datasets, probe spots, entities, barcodes, spatial projections, loci, reference sequences, sequencing and/or biological samples disclosed herein, or any substitutions, modifications, additions, deletions, and/or combinations thereof, as will be apparent to one skilled in the art.

As described above, in some embodiments, spatial sequencing for a biological sample (e.g., a tissue section) is performed by a method comprising obtaining barcoded nucleic acids (e.g., cDNA) from captured nucleic acid analytes (e.g.., RNA) using any of the sequencing methods disclosed herein. For example, in some implementations, sequencing libraries are prepared from captured nucleic acids and run on a sequencer to generate sequencing read data that is applied to a sequencing pipeline. Reads from the sequencer are grouped by barcodes and UMIs, and aligned to genes in a transcriptome reference, after which the pipeline generates a number of files, including a feature-barcode matrix. The barcodes correspond to individual spots within a capture area. The value of each entry in the spatial feature-barcode matrix is the number of analytes (e.g., RNA molecules) in proximity to (e.g., in contact with and/or captured by) the probe spot and/or capture probes affixed with that barcode, that align to a particular gene feature. Thus, sequencing data can be spatially positioned at probe spots in the capture area overlaid on the original biological sample. This enables users to observe patterns in feature abundance (e.g., gene or protein expression) in the spatial context of the one or more biological samples. In some embodiments, spatial sequencing is performed in accordance with the methods for spatial analysis of analytes disclosed above (see, for example, Definitions: (C) Methods for Spatial Analysis of Analytes, above).

Referring to Block 3104, in some embodiments, each locus in the plurality of loci is a respective gene in a plurality of genes, and each discrete attribute value in the corresponding plurality of discrete attribute values is a count of UMI that map to a corresponding probe spot and that also map to a respective gene in the plurality of genes.

Referring to Block 3106, in some embodiments, each locus in the plurality of loci is a respective feature in a plurality of features, each discrete attribute value in the corresponding plurality of discrete attribute values is a count of UMI that map to a corresponding probe spot and that also map to a respective feature in the plurality of features, and each feature in the plurality of features is an open-reading frame, an intron, an exon, an entire gene, an RNA transcript, a predetermined non-coding part of a reference genome, an enhancer, a repressor, a predetermined sequence encoding a variant allele, or any combination thereof.

Referring to Block 3108, in some embodiments, the plurality of loci comprises more than 1000 loci. In some embodiments, the plurality of loci comprises at least 100, at least 200, at least 300, at least 400, at least 500, at least 600, at least 700, at least 800, at least 900, at least 1000, at least 2000, at least 5000, at least 10,000, at least 20,000, at least 30,000, at least 40,000, at least 50,000, at least 100,000, at least 200,000, or at least 500,000 loci. In some embodiments, the plurality of loci comprises no more than 1 million, no more than 500,000, no more than 100,000, no more than 50,000, no more than 10,000, no more than 5000, or no more than 1000 loci. In some embodiments, the plurality of loci comprises from 100 to 5000, from 500 to 10,000, from 1000 to 100,000, from 2000 to 500,000, or from 100,000 to 1 million loci. In some embodiments, the plurality of loci falls within another range starting no lower than 100 loci and ending no higher than 1 million loci.

Referring to Block 3112, in some embodiments, each unique barcode in the plurality of barcodes encodes a unique predetermined value selected from the set {1, ..., 1024}, {1, ..., 4096}, {1, ..., 16384}, {1, ..., 65536}, {1, ..., 262144}, {1, ..., 1048576}, {1, ...,4194304}, {1, ..., 16777216}, {1, ..., 67108864}, or {1, ...,1 x 10¹²}.

Referring to Block 3114, in some embodiments, the plurality of loci include one or more loci on a first chromosome and one or more loci on a second chromosome other than the first chromosome.

Referring to Block 3116, in some embodiments, a file size of the discrete attribute value dataset is more than 100 megabytes. For instance, as described above, in some embodiments, a discrete attribute value dataset 120 has a file size of more than 1 megabytes, more than 5 megabytes, more than 100 megabytes, more than 500 megabytes, or more than 1000 megabytes. In some embodiments, a discrete attribute value dataset 120 has a file size of between 0.5 gigabytes and 25 gigabytes. In some embodiments, a discrete attribute value dataset 120 has a file size of between 0.5 gigabytes and 100 gigabytes.

Referring to Block 3118, in some embodiments, the discrete attribute value dataset represents a whole transcriptome sequencing experiment that quantifies gene expression in counts of transcript reads mapped to the plurality of genes. Referring to Block 3120, in some embodiments, the discrete attribute value dataset represents a targeted transcriptome sequencing experiment that quantifies gene expression in UMI counts mapped to probes in the plurality of probe spots.

In some embodiments, analysis of the discrete attribute value dataset, including clustering, visualization, indexing, and/or displaying, comprises any one or more of the embodiments for analysis of discrete attribute value datasets disclosed herein, including clustering, visualization, indexing, and/or displaying, or any substitutions, modifications, additions, deletions, and/or combinations thereof, as will be apparent to one skilled in the art.

For instance, referring to Block 3122, in some embodiments, the obtaining comprises clustering all or a subset of the probe spots in the plurality of probe spots across the one or more spatial projections using the discrete attribute values assigned to each respective probe spot in each of the one or more spatial projections as a multi-dimensional vector thereby forming a plurality of clusters.

Referring to Block 3126, in some embodiments, each respective cluster in the plurality of clusters consists of a unique subset of the plurality of probe spots. Referring to Block 3128, in some embodiments, at least one probe spot in the plurality of probe spots is assigned to more than one cluster in the plurality of clusters with a corresponding probability value indicating a probability that the at least one probe spot belongs to a respective cluster of the plurality of clusters. Referring to Block 3130, in some embodiments, the clustering all or a subset of the probe spots comprises k-means clustering with K set to a predetermined value between one and twenty-five.

Referring to Block 3132, in some embodiments, the probe spots of a first cluster in the plurality of cluster are predominantly a first cell type and cells in the first tissue section that map to the probe spots of a second cluster in the plurality of clusters are a second cell type. For instance, in some embodiments, the first cell type is diseased cells, and the second cell type is lymphocytes.

In some embodiments, a respective cluster is predominantly a first cell type when at least 30%, at least 40%, at least 50%, at least 55%, at least 60%, at least 65%, at least 70%, at least 75%, at least 80%, at least 85%, at least 90%, at least 95%, at least 97%, at least 98%, or at least 99% of the entities represented by the respective cluster are of the first cell type.

Referring to Block 3134, the method further includes indexing a two-dimensional spatial arrangement of the plurality of probe spots, in which each respective probe spot in the plurality of probe spots is independently assigned a unique two-dimensional position, in a k-dimensional binary search tree. Referring to Block 3136, the method further comprises displaying the two-dimensional spatial arrangement of the plurality of probe spots on the display in accordance with a first spatial projection in the one or more spatial projections. Referring to Block 3138, in some embodiments, the one or more spatial projections is a plurality of spatial projections of the biological sample, the plurality of spatial projections comprises the first spatial projection for the first tissue section of the biological sample, and the plurality of spatial projections comprises a second spatial projection for a second tissue section of the biological sample.

Referring to Blocks 3140, 3142, 3144, and 3146, the method further includes receiving a user selection of a subset of the two-dimensional spatial arrangement on the display, determining each probe spot in the plurality of probe spots that is a member of the subset using the k-dimensional binary search tree, thereby identifying a subset of probe spots in the plurality of probe spots, assigning each probe spot in the subset of probe spots a user provided category, and modifying the discrete attribute value dataset to store an association of each respective probe spot in the subset of probes spots to the user provided category.

In some embodiments, selection of two-dimensional spatial arrangements, including user selection, search trees, category assignment, and modification of displays, comprises any one or more of the embodiments for subset selection disclosed herein, including user selection, search trees, category assignment, and modification of displays, or any substitutions, modifications, additions, deletions, and/or combinations thereof, as will be apparent to one skilled in the art.

Another aspect of the present disclosure provides a visualization system comprising one or more processing cores, a memory, and a display, the memory storing instructions for performing a method for evaluating a first tissue section of a biological sample, the method comprising: obtaining a discrete attribute value dataset associated with a plurality of probe spots (e.g., at least 100,000 probe spots), where each probe spot in the plurality of probe spots is assigned a unique barcode in a plurality of barcodes, the discrete attribute value dataset comprising: (i) one or more spatial projections of the biological sample, and (ii) a corresponding plurality of discrete attribute values (e.g., at least 500 discrete attribute values) for each respective probe spot in the plurality of probe spots obtained from spatial sequencing of the first tissue section, where each respective discrete attribute value in the corresponding plurality of discrete attribute values is for a different loci in a plurality of loci; displaying the plurality of probe spots on the display in a two-dimensional spatial arrangement in accordance with a first spatial projection in the one or more spatial projections, with each respective probe spot in the plurality of probe spots independently assigned a unique two-dimensional position in the two-dimensional spatial arrangement; receiving a user selection of a subset of the two-dimensional spatial arrangement on the display; responsive to the user selection, creating a data structure that comprises the unique two-dimensional position of each probe spot in the subset of probe spots in the two-dimensional spatial arrangement; and submitting the data structure to the graphics processing unit with a uniform, thereby recoloring the subset of probe spots on the display in accordance with the uniform.

In some embodiments, the foregoing aspect comprises any one or more of the embodiments disclosed herein, including biological samples, discrete attribute value datasets, probe spots, barcodes, spatial projections, sequencing, loci, spatial arrangements, visualization, subset selection, data structure generation, graphics processing units and/or uniforms, or any substitutions, modifications, additions, deletions, and/or combinations thereof, as will be apparent to one skilled in the art. For example, in some embodiments, spatial sequencing is performed in accordance with any of the methods for spatial sequencing and/or spatial analysis of analytes disclosed above (see, for example, Definitions: (C) Methods for Spatial Analysis of Analytes, above).

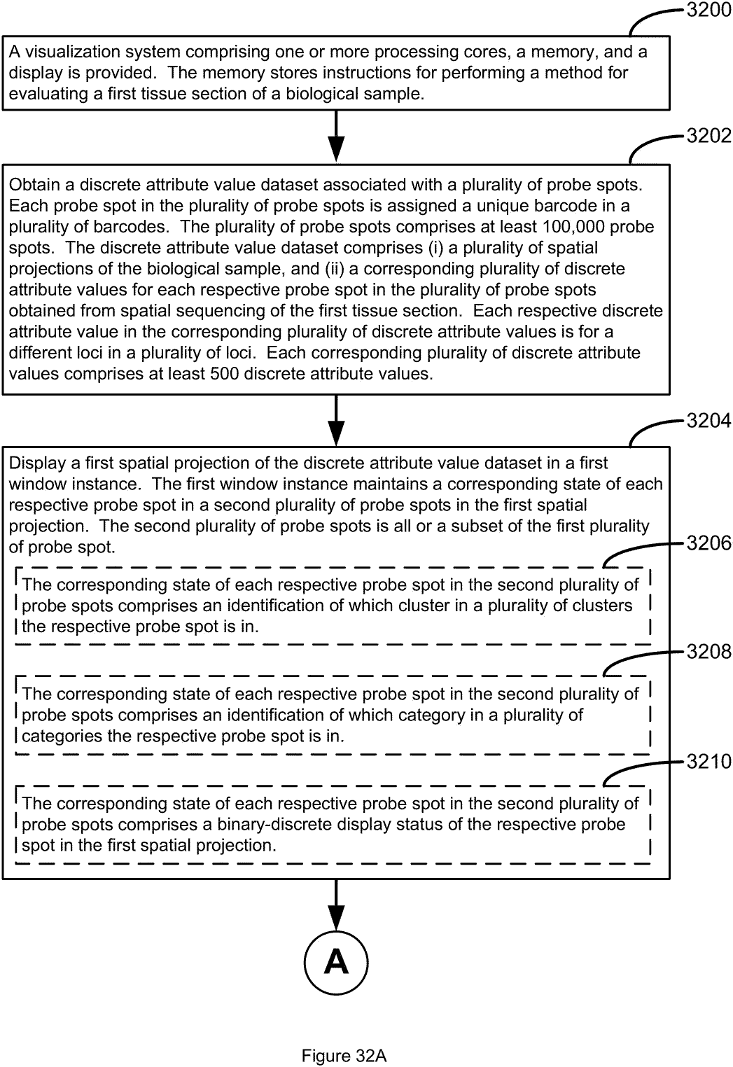

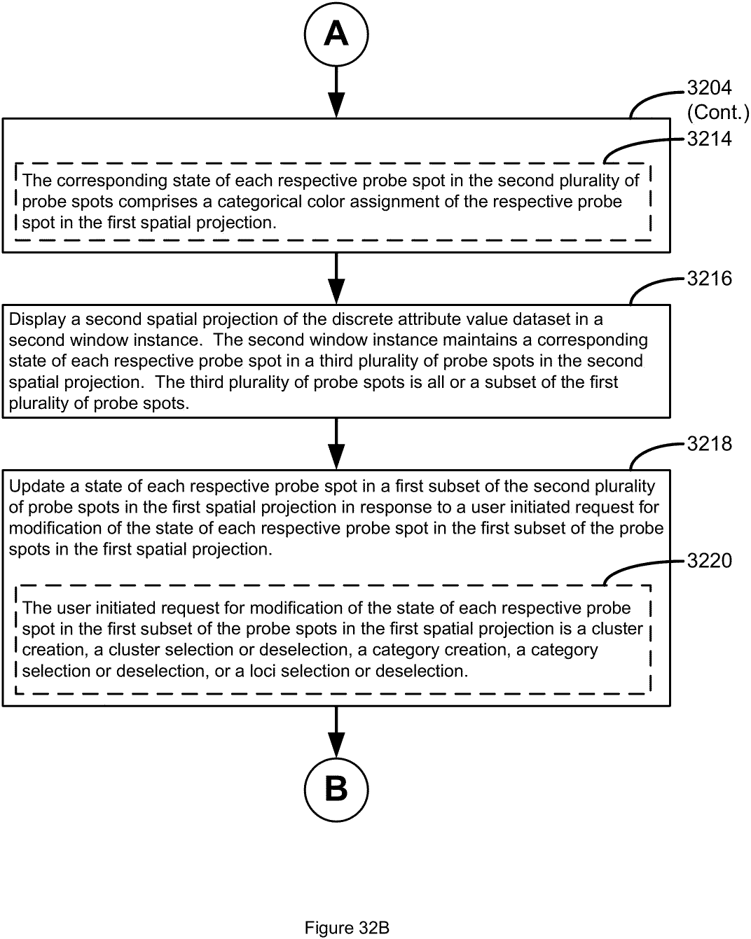

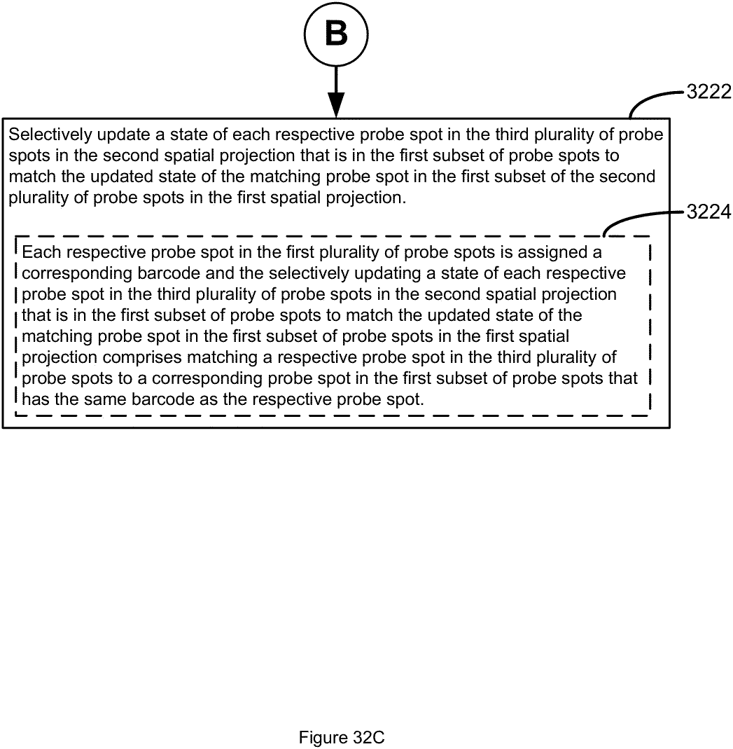

Referring to Block 3200 of FIGS. 32A-C, the present disclosure further provides a visualization system comprising one or more processing cores, a memory, and a display, the memory storing instructions for performing a method for evaluating a first tissue section of a biological sample.

Referring to Block 3202, the method comprises obtaining a discrete attribute value dataset associated with a plurality of probe spots (e.g., at least 100,000 probe spots), where each probe spot in the plurality of probe spots is assigned a unique barcode in a plurality of barcodes, the discrete attribute value dataset comprising (i) a plurality of spatial projections of the biological sample, and (ii) a corresponding plurality of discrete attribute values (e.g., at least 500 discrete attribute values) for each respective probe spot in the plurality of probe spots obtained from spatial sequencing of the first tissue section, where each respective discrete attribute value in the corresponding plurality of discrete attribute values is for a different loci in a plurality of loci.

The method further includes, referring to Block 3204, displaying a first spatial projection of the discrete attribute value dataset in a first window instance, where the first window instance maintains a corresponding state of each respective probe spot in a second plurality of probe spots in the first spatial projection, where the second plurality of probe spots is all or a subset of the first plurality of probe spots.

In some embodiments, referring to Block 3206, the corresponding state of each respective probe spot in the second plurality of probe spots comprises an identification of which cluster in a plurality of clusters the respective probe spot is in. Referring to Block 3208, in some embodiments, the corresponding state of each respective probe spot in the second plurality of probe spots comprises an identification of which category in a plurality of categories the respective probe spot is in. Referring to Block 3210, in some embodiments, the corresponding state of each respective probe spot in the second plurality of probe spots comprises a binary-discrete display status of the respective probe spot in the first spatial projection. Referring to Block 3214, in some embodiments, the corresponding state of each respective probe spot in the second plurality of probe spots comprises a categorical color assignment of the respective probe spot in the first spatial projection.

Referring to Blocks 3216 and 3218, the method further includes displaying a second spatial projection of the discrete attribute value dataset in a second window instance, where the second window instance maintains a corresponding state of each respective probe spot in a third plurality of probe spots in the second spatial projection, where the third plurality of probe spots is all or a subset of the first plurality of probe spots. A state of each respective probe spot in a first subset of the second plurality of probe spots in the first spatial projection is updated in response to a user initiated request for modification of the state of each respective probe spot in the first subset of the probe spots in the first spatial projection.

Referring to Block 3220, in some embodiments, the user initiated request for modification of the state of each respective probe spot in the first subset of the probe spots in the first spatial projection is a cluster creation, a cluster selection or deselection, a category creation, a category selection or deselection, or a loci selection or deselection.

Referring to Block 3222, the method further includes selectively updating a state of each respective probe spot in the third plurality of probe spots in the second spatial projection that is in the first subset of probe spots to match the updated state of the matching probe spot in the first subset of the second plurality of probe spots in the first spatial projection. Referring to Block 3224, in some embodiments, each respective probe spot in the first plurality of probe spots is assigned a corresponding barcode and the selectively updating a state of each respective probe spot in the third plurality of probe spots in the second spatial projection that is in the first subset of probe spots to match the updated state of the matching probe spot in the first subset of probe spots in the first spatial projection comprises matching a respective probe spot in the third plurality of probe spots to a corresponding probe spot in the first subset of probe spots that has the same barcode as the respective probe spot.

In some embodiments, the foregoing aspect comprises any one or more of the embodiments disclosed herein, including biological samples, discrete attribute value datasets, probe spots, barcodes, spatial projections, sequencing, loci, spatial arrangements, visualization, clustering, windows, category assignments, and/or state modifications, or any substitutions, modifications, additions, deletions, and/or combinations thereof, as will be apparent to one skilled in the art.

### Additional Embodiments

Another aspect of the present disclosure provides a method of evaluating one or more biological samples and/or a first tissue section of a biological sample, using any of the systems disclosed herein.

Another aspect of the present disclosure provides a computing system comprising at least one processor and memory storing at least one program to be executed by the at least one processor, the at least one program comprising instructions for evaluating one or more biological samples and/or a first tissue section of a biological sample by any of the methods disclosed herein.

Still another aspect of the present disclosure provides a non-transitory computer readable storage medium storing one or more programs for evaluating one or more biological samples and/or a first tissue section of a biological sample. The one or more programs are configured for execution by a computer. The one or more programs collectively encode computer executable instructions for performing any of the methods disclosed herein.

For instance, one aspect of the present disclosure provides a computer-readable storage medium storing one or more computer programs, the one or more computer programs comprising instructions that, when executed by an electronic device with one or more processors and a memory, cause the electronic device to perform a method for evaluating one or more biological samples, comprising: obtaining a discrete attribute value dataset derived by nucleic acid sequencing (e.g., single cell or single nuclei sequencing) of the one or more biological samples, wherein the discrete attribute value dataset comprises a corresponding discrete attribute value for each reference sequence in a plurality of reference sequences for each respective entity in a plurality of entities in the one or more biological samples, wherein the plurality of entities comprises 100,000 entities; indexing a two-dimensional spatial arrangement of the plurality of entities, in which each respective entity in the plurality of entities is independently assigned a unique two-dimensional position, in a k-dimensional binary search tree; displaying the two-dimensional spatial arrangement of the plurality of entities on the display; receiving a user selection of a subset of the two-dimensional spatial arrangement on the display; determining each entity in the plurality of entities that is a member of the subset using the k-dimensional binary search tree, thereby identifying a subset of entities; assigning each entity in the subset of entities to a user provided category; and modifying the discrete attribute value dataset to store an association of each respective entity in the subset of entities to the user provided category.

Another aspect of the present disclosure provides a method of evaluating one or more biological samples, the method comprising, using a computer system comprising one or more processing cores, a memory, and a display: obtaining a discrete attribute value dataset derived by nucleic acid sequencing (e.g., single cell or single nuclei sequencing) of the one or more biological samples, wherein the discrete attribute value dataset comprises a corresponding discrete attribute value for each reference sequence in a plurality of reference sequences for each respective entity in a plurality of entities in the biological sample, wherein the plurality of entities comprises 100,000 entities; indexing a two-dimensional spatial arrangement of the plurality of entities, in which each respective entity in the plurality of entities is independently assigned a unique two-dimensional position, in a k-dimensional binary search tree; displaying the two-dimensional spatial arrangement of the plurality of entities on the display; receiving a user selection of a subset of the two-dimensional spatial arrangement on the display; determining each entity in the plurality of entities that is a member of the subset using the k-dimensional binary search tree, thereby identifying a subset of entities; assigning each entity in the subset of entities to a user provided category; and modifying the discrete attribute value dataset to store an association of each respective entity in the subset of entities to the user provided category.

Yet another aspect of the present disclosure provides a computer-readable storage medium storing one or more computer programs, the one or more computer programs comprising instructions that, when executed by an electronic device with one or more processors and a memory, cause the electronic device to perform a method for evaluating one or more biological samples, comprising: obtaining a discrete attribute value dataset derived by nucleic acid sequencing (e.g., single cell or single nuclei sequencing) of the one or more biological samples, wherein the discrete attribute value dataset comprises a corresponding discrete attribute value for each reference sequence in a plurality of reference sequences for each respective entity in a plurality of entities in the biological sample, wherein the plurality of entities comprises 100,000 entities; displaying the plurality of entities on the display in a two-dimensional spatial arrangement in which each respective entity in the plurality of entities is independently assigned a unique two-dimensional position; receiving a user selection of a subset of the two-dimensional spatial arrangement on the display; responsive to the user selection, creating a data structure that comprises the unique two-dimensional position of each entity in the subset of cells in the two-dimensional spatial arrangement; and submitting the data structure to the graphics processing unit with a uniform, thereby recoloring the subset of entities on the display in accordance with the uniform.

An additional aspect of the present disclosure provides a computer-readable storage medium storing one or more computer programs, the one or more computer programs comprising instructions that, when executed by an electronic device with one or more processors and a memory, cause the electronic device to perform a method for evaluating one or more biological samples, comprising: obtaining a discrete attribute value dataset derived by nucleic acid sequencing (e.g., single cell or single nuclei sequencing) of the one or more biological samples, wherein the discrete attribute value dataset comprises a corresponding discrete attribute value for each reference sequence in a plurality of reference sequences for each respective entity in a first plurality of entities in the one or more biological samples, wherein the first plurality of entities comprises 100,000 entities; displaying a first spatial projection of the discrete attribute value dataset in a first window instance, wherein the first window instance maintains a corresponding state of each respective entity in a second plurality of entities in the first spatial projection, wherein the second plurality of entities is all or a subset of the first plurality of entities; displaying a second spatial projection of the discrete attribute value dataset in a second window instance, wherein the second window instance maintains a corresponding state of each respective entity in a third plurality of entities in the second spatial projection, wherein the third plurality of entities is all or a subset of the first plurality of entities; updating a state of each respective entity in a first subset of the second plurality of entities in the first spatial projection in response to a user initiated request for modification of the state of each respective entity in the first subset of the entities in the first spatial projection; and selectively updating a state of each respective entity in the third plurality of entities in the second spatial projection that is in the first subset of entities to match the updated state of the matching entities in the first subset of the second plurality of entities in the first spatial projection.

Still another aspect of the present disclosure provides a computer-readable storage medium storing one or more computer programs, the one or more computer programs comprising instructions that, when executed by an electronic device with one or more processors and a memory, cause the electronic device to perform a method for evaluating one or more biological samples, comprising: obtaining a discrete attribute value dataset associated with a plurality of probe spots, wherein each probe spot in the plurality of probe spots is assigned a unique barcode in a plurality of barcodes and the plurality of probe spots comprises at least 100,000 probe spots, the discrete attribute value dataset comprising: (i) one or more spatial projections of the biological sample, and (ii) a corresponding plurality of discrete attribute values for each respective probe spot in the plurality of probe spots obtained from spatial sequencing of the first tissue section, wherein each respective discrete attribute value in the corresponding plurality of discrete attribute values is for a different loci in a plurality of loci and each corresponding plurality of discrete attribute values comprises at least 500 discrete attribute values; indexing a two-dimensional spatial arrangement of the plurality of probe spots, in which each respective probe spot in the plurality of probe spots is independently assigned a unique two-dimensional position, in a k-dimensional binary search tree; displaying the two-dimensional spatial arrangement of the plurality of probe spots on the display in accordance with a first spatial projection in the one or more spatial projections; receiving a user selection of a subset of the two-dimensional spatial arrangement on the display; determining each probe spot in the plurality of probe spots that is a member of the subset using the k-dimensional binary search tree, thereby identifying a subset of probe spots in the plurality of probe spots; assigning each probe spot in the subset of probe spots a user provided category; and modifying the discrete attribute value dataset to store an association of each respective probe spot in the subset of probes spots to the user provided category.

Another aspect of the present disclosure provides a computer-readable storage medium storing one or more computer programs, the one or more computer programs comprising instructions that, when executed by an electronic device with one or more processors and a memory, cause the electronic device to perform a method for evaluating one or more biological samples, comprising: obtaining a discrete attribute value dataset associated with a plurality of probe spots, wherein each probe spot in the plurality of probe spots is assigned a unique barcode in a plurality of barcodes and the plurality of probe spots comprises at least 100,000 probe spots, the discrete attribute value dataset comprising: (i) a plurality of spatial projections of the biological sample, and (ii) a corresponding plurality of discrete attribute values for each respective probe spot in the plurality of probe spots obtained from spatial sequencing of the first tissue section, wherein each respective discrete attribute value in the corresponding plurality of discrete attribute values is for a different loci in a plurality of loci and each corresponding plurality of discrete attribute values comprises at least 500 discrete attribute values; displaying a first spatial projection of the discrete attribute value dataset in a first window instance, wherein the first window instance maintains a corresponding state of each respective probe spot in a second plurality of probe spots in the first spatial projection, wherein the second plurality of probe spots is all or a subset of the first plurality of probe spots; displaying a second spatial projection of the discrete attribute value dataset in a second window instance, wherein the second window instance maintains a corresponding state of each respective probe spot in a third plurality of probe spots in the second spatial projection, wherein the third plurality of probe spots is all or a subset of the first plurality of probe spots; updating a state of each respective probe spot in a first subset of the second plurality of probe spots in the first spatial projection in response to a user initiated request for modification of the state of each respective probe spot in the first subset of the probe spots in the first spatial projection; and selectively updating a state of each respective probe spot in the third plurality of probe spots in the second spatial projection that is in the first subset of probe spots to match the updated state of the matching probe spot in the first subset of the second plurality of probe spots in the first spatial projection.

### Example User Interface for Visualization System

Now that the overall functionality of the systems and methods of the present disclosure has been introduced, attention turns to additional features afforded by the present disclosure. As illustrated in FIG. 5, the lower panel 502 is arranged by rows and columns. Each row corresponds to a different reference sequence (e.g., locus). Each column corresponds to a different cluster. Each cell, then, illustrates the fold change (e.g., log₂ fold change) of the average discrete attribute value 124 for the reference sequence 122 represented by the row the cell is in across the entities 126 of the cluster represented by the column the cell is in compared to the average discrete attribute value 124 of the respective reference sequence 122 in the entities in the remainder of the clusters represented by the discrete attribute value dataset 120.

The lower panel 502 has two settings. The first is a hierarchical clustering view of significant loci 122 per cluster. In some embodiments, log₂ fold change in expression refers to the log₂ fold value of (i) the average number of transcripts (discrete attribute value) measured in each of the entities of the subject cluster that map to a particular gene (reference sequence 122) and (ii) the average number of transcripts measured in each of the entities of all clusters other than the subject cluster that map to the particular gene.

In some embodiments, selection of a particular reference sequence (row) in the lower panel 502 of FIG. 5 causes the reference sequence (feature) associated with that row to be an active feature that is posted to the active feature list 506. For instance, as illustrated in FIG. 5, the reference sequence “CCDC80” from lower panel 502 has been selected and so the reference sequence “CCDC80” is in the active feature list 506. The active feature list 506 is a list of all features that a user has either selected (e.g., “CCDC80”) or uploaded. The expression patterns of those features are displayed in panel 504 of FIG. 5. If more than one feature is in the active feature list 506, then the expression patter that is displayed in panel 504 corresponds to a combination (measure of central tendency) of all the features. If only one feature is presented or selected in the active feature list 506, then the expression pattern that is overlayed on the native spatial arrangement 125 in panel 504 is the expression pattern corresponding to the selected feature. Thus, in FIG. 5, each respective entity in the discrete attribute value dataset 120, regardless of which cluster the entity is in, is illuminated with an intensity, color, or other form of display attribute that is commensurate with a number of transcripts (e.g., log₂ of expression) of the single active feature CCDC80 that is present in the respective entity 126 in the upper panel 504.

At the bottom of the active feature list 506 there are a number of options that control how the data is visualized in the upper panel 504. The scale & attribute parameters 510 control how the expression patterns are rendered in the upper panel 504. For instance, toggle, 512 sets which scale value to display (e.g., Log₂, linear, log-normalized). The top right menu sets how to combine values when there are multiple features in the Active Feature List. For instance, in the case where two features (e.g., loci) have been selected for the active feature list 506, toggle 514 can be used to display, in each entity, the feature minimum, feature maximum, feature sum, or feature average. Thus, consider the case where features (e.g., loci) A and B are selected as the active features for the active feature list 506. In this case, selection of “feature minimum” will cause each respective entity to be assigned a color on the color scale that is commensurate of a minimum expression value, that is, the expression of A or the expression of B, whichever is lower. Thus, each respective entity is independently evaluated for the expression of A and B at the respective entity, and the entity is colored by the lowest expression value of A and B. On the other hand, toggle 514 can be used to select the maximum feature value from among the features in the active feature list 506 for each entity, or to sum the feature values across the features in the active feature list 506 for each entity or to provide a measure of central tendency, such as average, across the features in the active feature list 506 for each entity.

The select by count menu options 516 control how to filter the expression values displayed. For instance, the color palette 510 controls the color scale and range of values. The user can also choose to manually set the minimum and maximum of the color scale by unchecking an Auto-scale checkbox (not shown), typing in a value, and clicking an Update Min/Max button (not shown). When setting manual minimum and maximum values, entities with values outside the range, less than the minimum or greater than the maximum, are colored gray. This is particularly useful if there is a high level of noise or ambient expression of a reference sequence or a combination of reference sequences in the active feature list 506. Increasing the minimum value of the scale filters that noise. It is also useful to configure the scale to optimally highlight the expression of genes of interest.

In FIG. 5, color scale 508 shows the Log₂ expression of CCDC80 ranging from 0.0 to 5.0. In this way, a user can quickly ascertain the relative expression of a specific reference sequence in the entities of the discrete attribute dataset 120. Moreover, the present disclosure is not limited to showing the Log₂ relative expression of a reference sequence. In some embodiments, toggle 510 can be used to illustrate the relative expression of features in the active feature list 506 on a linear basis or a log-normalized basis. Moreover, palette 510 can be used to change the color scale 508 to other colors, as well as to set the minimum and maximum values that are displayed.

Toggle 518 is used to toggle between “Gene/Feature Expression” mode, “Categories” mode, and “Filters” mode.

In “Gene/Feature Expression” mode, the user can control the content in the mode panel 520 of the active feature list 506 by clicking on affordance 522. This allows the user to select from among a “new list” option, an “edit name” option, a “delete list” option, and an “import list” option. The “new list” option is used to create a custom list of features to visualize. The “edit name” option is used to edit the name of the active feature list. The “delete list” option is used to delete an active feature list. The “import list” option is used to import an active feature list from an external source while the “new list” option is used to create a custom list of features to visualize.

When toggle 518 is switched to “Filters” mode, the user can compose complex Boolean filters to find barcodes that fulfill selection criteria. For instance, the user can create rules based on feature counts or cluster membership and combine these rules using Boolean operators. The user can then save and load filters and use them across multiple datasets.

Panel 502 of FIG. 5 provides a tabular representation of the log₂ discrete attribute values 124 in column format, whereas the heat map of FIG. 4 showed the log₂ discrete attribute values 124 in rows. The user can select any respective cluster 158 by selecting the column label for the respective cluster. This will re-rank all the reference sequences 122 such that those reference sequences that are associated with the most significant discrete attribute value 124 in the selected cluster 158 are ranked first (e.g., in the order of the most reference sequences having the most significant associated discrete attribute value 124). Moreover, a p-value is provided for the discrete attribute value of each reference sequence 122 in the selected cluster to provide the statistical significance of the discrete attribute value 124 in the selected cluster 158 relative to the discrete attribute value 124 of the same reference sequence 122 in all the other clusters 158. In some embodiments, these p-values are calculated based upon the absolute discrete attribute values 124, not the log₂ values used for visualization in the heat map 402. Referring to FIG. 5 to illustrate, the reference sequence 122 in cluster 1 that has the largest associated discrete attribute value 124, ACKR1, has a p-value of 4.62e⁻⁷⁴. As illustrated in FIG. 5, this p-value is annotated with a star system, in which four stars means there is a significant difference between the selected cluster (k-means cluster 158-1 in FIG. 5) and the rest of the clusters for a given reference sequence, whereas fewer stars means that there is a less significant difference in the discrete attribute value 124 (e.g., difference in expression) between the reference sequence 122 in the selected cluster relative to all the other clusters. By clicking a second time on the selected cluster 158 label, the ranking of the entire table is inverted so that the reference sequence 122 associated with the least significant discrete attribute value 124 (e.g., least expressed) is at the top of the table. Selection of the label for another cluster (e.g., cluster 158-9) causes the entire table 502 to re-rank based on the discrete attribute values 124 of the reference sequences 122 in the entities that are in k-means cluster for the associated cluster associated with (e.g., cluster 158-9). In this way, the sorting is performed to more easily allow for the quantitative inspection of the difference in discrete attribute value 158 in any one cluster 158 relative to the rest of the clusters.

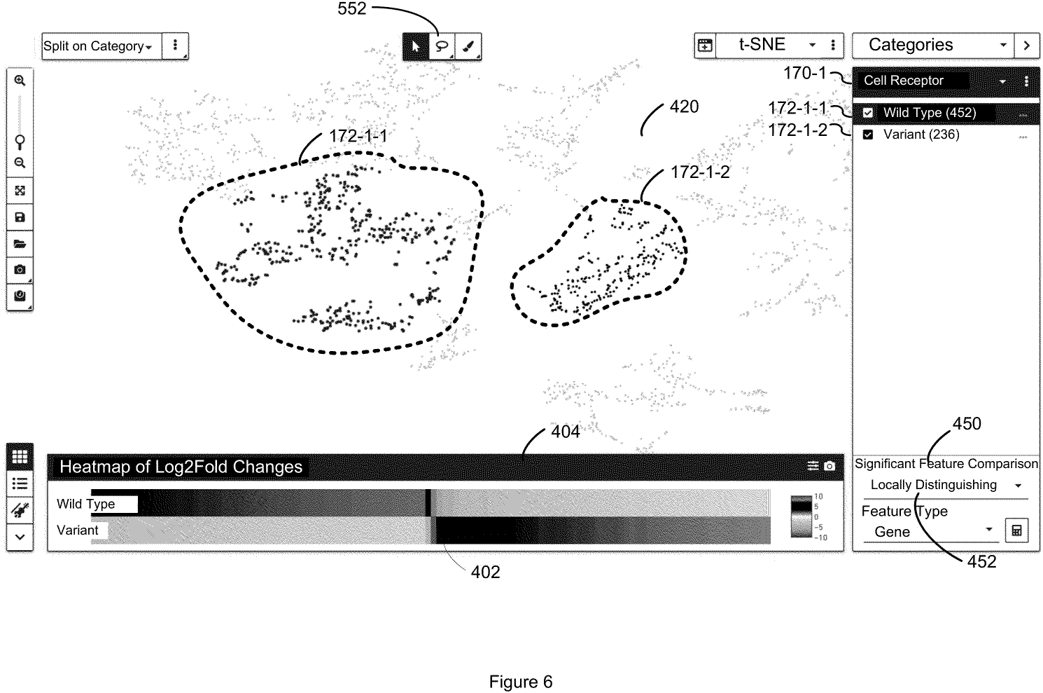

As illustrated by tab 552 of FIG. 5, the table of values 502 can be exported, e.g., to an EXCEL .csv file, by pressing tab 552 at which point the user is prompted to save the table as a csv (or other file format). In this way, once the user has completed their exploration of the k-means clustering, tab 552 allows the user to export the values. Moreover, the user is given control over which values to export (e.g., top 10 reference sequences, top 20 reference sequences, top 50 reference sequences, top 100 reference sequences, where “top” is from the frame of reference of the cluster the user has identified in panel 502. Thus, if the user has selected column 158-1 and “top 50 reference sequences,” the discrete attribute values 124 of the top 50 reference sequences in cluster 1 will be selected for exporting and what will be exported will be the discrete attribute values 124 of these 50 reference sequences in each of the clusters of the discrete attribute value dataset (clusters 1-11 in the example dataset used for FIGS. 5 and 6). Moreover, in some embodiments, a user is able to load and save lists of reference sequences to and from persistent storage, for instance, using panel 404.

Moreover, in some embodiments, a user is able to select entities using the selection tools 552. Once the entities are selected the user can assign the selection a category name, assign the entities to a particular cluster or un-assign the selected entities from all clusters. Further, the user can export the top reference sequences in the selected entities using the affordance 552 in the manner described above for clusters 158.

Referring to FIG. 4, the heat map 402 provides a log₂ differential that is optimal where the discrete attribute value 124 represents the number of transcripts that map to a given entity in order to provide a sufficient dynamic range over the number of transcripts seen per gene in the given entity. Referring to FIG. 5, toggle 554 provides pop-menu 556 which permits the user to toggle between the fold change and the median-normalized (centered) average discrete attribute value 124 per reference sequence 122 per entity in each cluster 158 (e.g., the number of transcripts per entity). Thus, in FIG. 5, for Gene ACKR1 the log2 fold change of the transcripts of ACKR1 in the entities of cluster 158-1 relative to all other clusters is 2.17, the Log2 fold change of the transcripts of ACKR1 in the entities of cluster 158-2 relative to all other clusters is -1.94, and so forth. Further menu 556 can be used to display the mean-normalized average value of ACKR1 in each of the clusters, as well as the mean-normalized average value of other reference sequences that are represented in the discrete attribute value dataset 120. In some embodiments, the average value is some other measure of central tendency of the discrete attribute value 124 such as an arithmetic mean, weighted mean, midrange, midhinge, trimean, Winsorized mean, median, or mode of all the discrete attribute values 124 for the reference sequence 122 measured in each of the entities in the respective cluster 158. FIGS. 4 and 5 provides a means for discerning between those reference sequences 122 (e.g., genes) that are associated with significant average discrete attribute values 124 (e.g., fairly high transcript counts) in all the k-means clusters 158 and those reference sequences 122 (e.g., genes) that are associated with appreciable discrete attribute values 124 that localized to only certain k-means clusters.

### Example 1

Referring to FIG. 6, an example visualization system 100 comprising a plurality of processing cores, a persistent memory, and a non-persistent memory was used to perform a method for visualizing a pattern in a dataset. In this example, the example visualization system 100 was a DELL Inspiron 17 7000 with MICROSOFT WINDOWS 10 PRO, 16.0 gigabytes of RAM memory, and Intel i7-8565U CPM operating at 4.50 gigahertz with 4 cores and 8 logical processors with the visualization module 119 installed. The discrete attribute value dataset 120, comprising a single spatial image 125 of a tissue sample with accompanying discrete attribute values 124 for hundreds of loci at each of hundreds of probe spots 126 was stored in persistent memory. The dataset was clustered prior to loading onto the example computer system 100, using principal components derived from the discrete attribute values across each locus in the plurality of loci across each probe spot 126 in the plurality of probe spots thereby assigning each respective probe spot in the plurality of probe spots to a corresponding cluster in a plurality of clusters. These cluster assignments were already assigned prior to loading the dataset into the example computer system 100. Each respective cluster in the plurality of clusters consisted of a unique different subset of the plurality of probe spots 123. For this example dataset 120, there were 8 clusters. Each respective cluster comprises a subset of the plurality of probe spots in a multi-dimensional space. This multi-dimensional space was compressed by t-SNE into two-dimensions for visualization in the upper panel 420.

Next, referring to FIG. 6, a new category, “Cell Receptor,” that was not in the loaded discrete attribute value dataset 120 was user defined by selecting a first class of probe spots 172-1-1 (“Wild Type”) using Lasso 552 and selecting displayed probe spots in the upper panel 420. A total of 452 probe spots 126 were selected from the Wild Type class. Further, a second class of probe spots 172-1-2 (“Variant”) was user defined using Lasso 552 to select the probe spots as illustrated in FIG. 6. Next, the loci whose discrete attribute values 124 discriminate between the identified user defined classes “Wild Type” and “Variant” were computed. For this, the locally distinguishing option 452 described above in conjunction with FIG. 4 was used to identify the loci whose discrete attribute values discriminate between class 172-1-1 (Wild Type) and class 172-1-2 (Variant). The Wild Type class consisted of whole transcriptome mRNA transcript counts for 452 probe spots. The Variant class consisted of whole transcriptome mRNA transcript counts for 236 probe spots. To do this, the differential value for each respective locus in the plurality of loci for class 172-1-1 was computed as a fold change in (i) a first measure of central tendency of the discrete attribute value for the respective locus measured in each of the probe spots in the plurality of probes spots in the class 172-1-1 and (ii) a second measure of central tendency of the discrete attribute value for the respective locus measured in each of the probe spots in the class 172-1-2. Then the heat map 402 of this computation for each of the loci was displayed in the lower panel 404 as illustrated in FIG. 6. In heat map 402, the first row represents the Wild Type class, and the second row represents the Variant class. Each column in the heat map shows the average expression of a corresponding gene across the probe spots of the corresponding class 172. The heat map includes more than 1000 different columns, each for a different human gene. The heat map shows which loci discriminate between the two classes. An absolute definition for what constitutes discrimination between the two classes is not provided because such definitions depend upon the technical problem to be solved. Moreover, those of skill in the art will appreciate that many such metrics can be used to define such discrimination and any such definition is within the scope of the present disclosure. Advantageously, the computation and display of the heat map 402 took less than two seconds on the example system using the disclosed clustering module 152.

Had more classes been defined, more computations would be needed. For instance, had there been a third class in this category and this third class selected, the computation of the fold change for each respective locus would comprise:
for the first class 172-1-1, computing (i) a first measure of central tendency of the discrete attribute value for the respective locus measured in each of the probe spots in the plurality of probe spots of the first class 172-1-1 and (ii) a second measure of central tendency of the discrete attribute value for the respective locus measured in each of the probe spots in the second 172-1-2 and third classes 172-1-3 collectively,
for the second class 172-1-1, computing (i) a first measure of central tendency of the discrete attribute value for the respective locus measured in each of the probe spots in the plurality of probe spots of the second class 172-1-2 and (ii) a second measure of central tendency of the discrete attribute value for the respective locus measured in each of the probe spots in the first class 172-1-1 and the third class 172-1-3 collectively, and
for the third class 172-1-3, computing (i) a first measure of central tendency of the discrete attribute value for the respective locus measured in each of the probe spots in the plurality of probe spots of the third class 172-1-3 and (ii) a second measure of central tendency of the discrete attribute value for the respective locus measured in each of the probe spots in the first class 172-1-1 and the second class 172-1-2 collectively.

### Example 2

Triple negative breast cancer (TNBC) accounts for 10-20% of all diagnosed breast cancer cases in the United States. TNBC is aggressive and exhibits poor prognosis due to resistance to traditional therapies. TNBC is complex, making it important to understand the underlying biology to improve outcomes.

Spatial transcriptomics technology has helped address the limitations of traditional pathological examination, combining the benefits of histological techniques and massive throughput of RNA-seq. Serial sections of TNBC were investigated using the 10X Genomics Visium Spatial Gene Expression Solution disclosed in the present disclosure, and also disclosed in U.S. Pat. Publication No. US 2021-0062272 entitled “Systems and Methods for Using the Spatial Distribution of Haplotypes to Determine a Biological Condition” which is hereby incorporated by reference, to resolve its tumorigenic expression profile. The assay in this example incorporates ~5000 molecularly barcoded, spatially encoded capture probes in probe spots 122 over which a tissue is placed, imaged, and permeabilized, capturing native mRNA in an unbiased fashion. Imaging and next-generation sequencing data were processed together resulting in gene expression mapped to image position. By capturing and sequencing of polyadenylated RNA transcripts from 10 µm thick sections of tissue combined with histological visualization of the tissue, the Visium platform generated an unbiased map of gene expression of cells within the native tissue morphology.

Through this, spatial patterns of gene expression were demonstrated that agreed with annotations from pathological examination combined with immunohistochemical staining for tumor infiltrating lymphocytes, a hallmark of TNBC. By aggregating data from serial sections, the delineation of gene expression patterns was improved and furthermore, improved statistical power for cell-type identification was demonstrated. This data was compared with 3’ single-nucleus RNA-seq data from the same sample, generating cell-type expression profiles that were used to estimate the proportion of cell-types observed at a given position. Furthermore, an enrichment strategy was used to select for cancer-associated genes using the cancer probes of Table 1 of U.S. Pat. Application No. 17/239,555, entitled “Capturing Targeted Genetic Targets Using a Hybridization/Capture Approach,” filed Apr. 24, 2021. The spatial patterns of gene expression using this pull-down approach showed concordance with the whole transcriptome assay, suggesting that a targeted transcriptome sequencing approach can be used where a fixed gene panel is appropriate.

Results from these efforts suggest that spatial gene expression profiling can provide a powerful complement to traditional histopathology, enabling both targeted panels and whole-transcriptome discovery of gene expression. This detailed view of the tumor microenvironment, as it varies across the tissue space, provides essential insight into disease understanding and the development of potential new therapeutic targets.

### Example 3

The gut microbiome, populated by trillions of microbes, interacts closely with the host’s cell system. Studies have revealed information about the average microbiota diversity and bacterial activity in the gut. However, this study of expression-based host-microbiome interactions in a spatial and high-throughput manner is a novel approach. Understanding the cartography of gene expression of host-microbiome interactions provides insights into the molecular basis and the widespread understanding of bacterial communication mechanisms. Using the techniques disclosed herein and as also described in U.S. Pat. Application No. 2021-0062272 entitled “Systems and Methods for Using The Spatial Distribution of Haplotypes to Determine a Biological Condition,” which is hereby incorporated by reference, a spatial transcriptomics method was developed that enables visualization and quantitative analysis of gene expression data directly from tissue sections by positioning the section on a barcoded array matrix. With this approach, both polyadenylated host and 16S bacterial transcripts are concurrently transcribed in situ and the spatial cDNAs are sequenced. More than 11,000 mouse genes were concurrently analyzed and more than nine bacterial families in the proximal and distal mouse colon were identified as a pilot study. The processing pipelines of the present disclosure were applied to determine spatial variance analysis across the collected tissue volume. This approach generated a large cell-interaction dataset with the ability to call changes significantly occurring in multiple host cell types dependent on the nearby microbiome composition. These findings demonstrate the power of spatially resolved, transcriptome-wide gene expression analysis identifying morphological patterns and thus understanding the molecular basis of host-microbiome interactions.

### Example 4

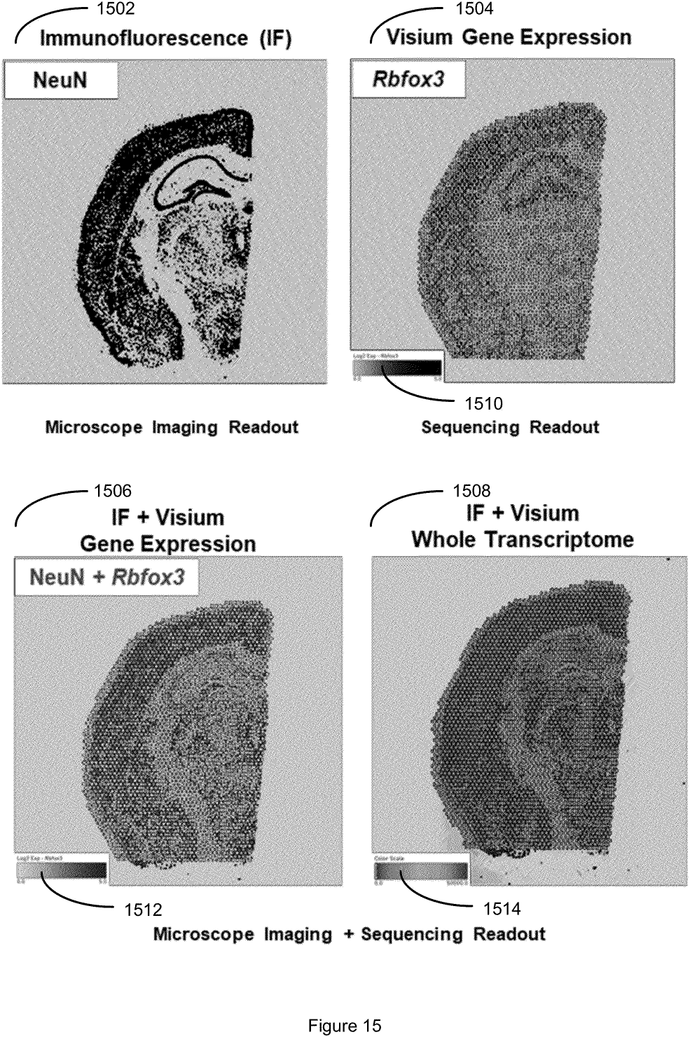

FIG. 15 illustrates an embodiment of the present disclosure in which a biological sample has an image 1502 that has been collected by immunofluorescence. Moreover, the sequence reads of the biological sample have been spatially resolved using the methods disclosed herein. More specifically, a plurality of spatial barcodes has been used to localize respective sequence reads in a plurality of sequence reads obtained from the biological sample (using the methods disclosed herein) to corresponding capture spots in a set of capture spots (through their spatial barcodes), thereby dividing the plurality of sequence reads into a plurality of subsets of sequence reads, each respective subset of sequence reads corresponding to a different capture spot (through their spatial barcodes) in the plurality of capture spots. As such, panel 1504 shows a representation of a portion (that portion that maps to the gene Rbfox3) of each subset of sequence reads at each respective position within image 1502 that maps to a respective capture spot corresponding to the respective position. Panel 1506 of FIG. 15 shows a composite representation comprising (i) the image 1502 and (ii) a representation of a portion (that portion that maps to the gene Rbfox3) of each subset of sequence reads at each respective position within image 1502 that maps to a respective capture spot corresponding to the respective position. Finally, panel 1508 of FIG. 15 shows a composite representation comprising (i) the image 1502 and (ii) a whole transcriptome representation of each subset of sequence reads at each respective position within image 1502 that maps to a respective capture spot corresponding to the respective position. In panels 1504, 1506, and 1508, each representation of sequence reads in each subset represents a number of unique UMI, on a capture spot by capture spot basis, in the subsets of sequence reads on a color scale basis as outlined by respective scales 1510, 1512, and 1514. While panel 1508 shows mRNA-based UMI abundance overlayed on a source images, the present disclosure can also be used to illustrate the spatial quantification of other analytes such as proteins, either superimposed on images of their source tissue or arranged in two-dimensional space using dimension reduction algorithms such as t-SNE or UMAP, including cell surface features (e.g., using the labelling agents described herein), mRNA and intracellular proteins (e.g., transcription factors), mRNA and cell methylation status, mRNA and accessible chromatin (e.g., ATAC-seq, DNase-seq, and/or MNase-seq), mRNA and metabolites (e.g., using the labelling agents described herein), a barcoded labelling agent (e.g., the oligonucleotide tagged antibodies described herein) and a V(D)J sequence of an immune cell receptor (e.g., T-cell receptor), mRNA and a perturbation agent (e.g., a CRISPR crRNA/sgRNA, TALEN, zinc finger nuclease, and/or antisense oligonucleotide as described herein). For general disclosure on how ATAC is spatially quantified using, for example clustering and/or t-SNE (where such cluster and/or t-SNE plots can be displayed in linked windows), see, United States Publication No. US-2020105373-A1 entitled “Systems and Methods for Cellular Analysis Using Nucleic Acid Sequencing” which is hereby incorporated by reference. For general disclosure on how V(D)J sequences are spatially quantified using, for example clustering and/or t-SNE (where such cluster and/or t-SNE plots can be displayed in linked windows), see, United States Patent Publication No. US 2018-0371545, entitled “Systems and Methods for Clonotype Screening,” which is hereby incorporated by reference.

### EXAMPLE 5 - Differential Values

In some embodiments, the techniques of this Example 5 are run on any of the discrete attribute value datasets of the present disclosure.

Once each probe spot 126 has been assigned to a respective cluster 158, the systems and methods of the present disclosure are able to compute, for each respective locus 122 in the plurality of loci for each respective cluster 158 in the plurality of clusters, a difference in the discrete attribute value 124 for the respective locus 122 across the respective subset of probe spots 126 in the respective cluster 158 relative to the discrete attribute value 124 for the respective locus 122 across the plurality of clusters 158 other than the respective cluster, thereby deriving a differential value 162 for each respective locus 122 in the plurality of loci for each cluster 158 in the plurality of clusters. For instance, in some such embodiments, a differential expression algorithm is invoked to find the top expressing genes that are different between probe spot classes or other forms of probe spot labels. This is a form of the general differential expressional problem in which there is one set of expression data and another set of expression data and the question to be addressed is determining which genes are differentially expressed between the datasets.

In some embodiments, differential expression is computed as the log₂ fold change in (i) the average number of transcripts (discrete attribute value 124 for locus 122) measured in each of the probe spots 126 of the subject cluster 158 that map to a particular gene (locus 122) and (ii) the average number of transcripts measured in each of the probe spots of all clusters other than the subject cluster that map to the particular gene. Thus, consider the case in which the subject cluster contains 50 probe spots and on average each of the 50 probe spots contain 100 transcripts for gene A. The remaining clusters collectively contain 250 probe spots and, on average, each of the 250 probe spots contains 50 transcripts for gene A. Here, the fold change in expression for gene A is 100/50 and the log₂ fold change is log₂(100/50) = 1. In FIG. 4, lower panel, the log₂ fold change is computed in this manner for each gene in the human genome.

In some embodiments, the differential value 162 for each respective locus 122 in the plurality of loci for each respective cluster 158 in the plurality of clusters is a fold change in (i) a first measure of central tendency of the discrete attribute value 124 for the locus measured in each of the probe spots 126 in the plurality of probe spots in the respective cluster 158 and (ii) a second measure of central tendency of the discrete attribute value 124 for the respective locus 122 measured in each of the probe spots 126 of all clusters 158 other than the respective cluster. In some embodiments, the first measure of central tendency is an arithmetic mean, weighted mean, midrange, midhinge, trimean, Winsorized mean, median, or mode of all the discrete attribute value 124 for the locus measured in each of the probe spots 126 in the plurality of probe spots in the respective cluster 158. In some embodiments, the second measure of central tendency is an arithmetic mean, weighted mean, midrange, midhinge, trimean, Winsorized mean, median, or mode of all the discrete attribute value 124 for the locus 122 measured in each of the probe spots 126 in the plurality of probe spots 126 in all clusters other than the respective cluster. In some embodiments, the fold change is a log₂ fold change. In some embodiments, the fold change is a log₁₀ fold change.

Given that measurement of discrete attribute values 124 for loci 122 (e.g., count of mRNA that maps to a given gene in a given probe spot) is typically noisy, the variance of the discrete attribute values 124 for loci 122 in each probe spot 126 (e.g., count of mRNA that maps to given gene in a given probe spot) in a given cluster 158 of such probe spots 126 is taken into account in some embodiments. This is analogous to the t-test, which is a statistical way to measure the difference between two samples. Here, in some embodiments, statistical methods that take into account that a discrete number of loci 122 are being measured (as the discrete attribute values 124 for a given locus 122) for each probe spot 126 and that model the variance that is inherent in the system from which the measurements are made are implemented.

Thus, in some embodiments, each discrete attribute value 124 is normalized prior to computing the differential value 162 for each respective locus 122 in the plurality of loci for each respective cluster 158 in the plurality of clusters. In some embodiments, the normalizing comprises modeling the discrete attribute value 124 of each locus associated with each probe spot in the plurality of probe spots with a negative binomial distribution having a consensus estimate of dispersion without loading the entire dataset into non-persistent memory 111. Such embodiments are useful, for example, for RNA-seq experiments that produce discrete attribute values 124 for loci 122 (e.g., digital counts of mRNA reads that are affected by both biological and technical variation). To distinguish the systematic changes in expression between conditions from noise, the counts are frequently modeled by the Negative Binomial distribution. See Yu, 2013, “Shrinkage estimation of dispersion in Negative Binomial models for RNA-seq experiments with small sample size,” Bioinformatics 29, pp. 1275-1282, which is hereby incoporated by reference.

The negative binomial distribution for a discrete attribute value 124 for a given locus 122 includes a dispersion parameter for the discrete attribute value 124, which tracks the extent to which the variance in the discrete attribute value 124 exceeds an expected value. See Yu, 2013, “Shrinkage estimation of dispersion in Negative Binomial models for RNA-seq experiments with small sample size,” Bioinformatics 29, pp. 1275-1282, and Cameron and Trivedi, 1998, “Regression Analysis of Count Data,” Econometric Society Monograph 30, Cambridge University Press, Cambridge, UK, each of which is hereby incorporated by reference. Rather than relying upon an independent dispersion parameter for the discrete attribute value 124 of each locus 122, some embodiments of the disclosed systems and methods advantageously use a consensus estimate across the discrete attribute values 124 of all the loci 122. This is termed herein the “consensus estimate of dispersion.” The consensus estimate of dispersion is advantageous for RNA-seq experiments in which whole transcriptome sequencing (RNA-seq) technology quantifies gene expression in biological samples in counts of transcript reads mapped to the genes, which is one form of experiment used to acquire the disclosed dicreate atribute values 124 in some embodiments, thereby concurrently quantifying the expression of many genes. The genes share aspects of biological and technical variation, and therefore a combination of the gene-specific estimates and of consensus estimates can yield better estimates of variation. See Yu, 2013, “Shrinkage estimation of dispersion in Negative Binomial models for RNA-seq experiments with small sample size,” Bioinformatics 29, pp. 1275-1282 and Anders and Huber, 2010, “Differential expression analysis for sequence count data,” Genome Biol 11, R106, each of which are hereby incorporated by reference. For instance, in some such embodiments, sSeq is applied to the discrete attribute value 124 of each locus 122. sSeq is disclosed in Yu, 2013, “Shrinkage estimation of dispersion in Negative Binomial models for RNA-seq experiments with small sample size,” Bioinformatics 29, pp. 1275-1282, which is hereby incorporated by reference. sSeq scales very well with the number of genes that are being compared. In typical experiments in accordance with the present disclosure, each cluster 158 may include hundreds, thousands, tens of thousands, hundreds of thousands, or more probe spots 126, and each respective probe spot 126 may contain mRNA expression data for hundreds, or thousands of different genes. As such, sSeq is particularly advantageous when testing for differential expression in such large discrete attribute value datasets 120. Of all the RNA-seq methods, sSeq is advantageously faster. Other single-probe spot differential expression methods exist and can be used in some embodiments, but they are designed for smaller-scale experiments. As such sSeq, and more generally techniques that normalize discrete attribute values by modeling the discrete attribute value 124 of each locus 122 associated with each probe spot 126 in the plurality of probe spots with a negative binomial distribution having a consensus estimate of dispersion without loading the entire discrete attribute value dataset 120 into non-persistent memory 111, are practiced in some embodiments, of the present disclosure. In some embodiments, in the case where parameters for the sSeq calculations are calculated, the discrete attribute values for each of the loci is examined in order to get a dispersion value for all the loci. Here, although all the discrete attribute values for the loci are accessed to make the calculation, the discrete attribute values are not all read from persistent memory 112 at the same time. In some embodiments, discrete attribute values are obtained by traversing through blocks of compressed data, a few blocks at a time. That is, a set of blocks (e.g., consisting of the few compressed blocks) in the dataset are loaded into non-persistent memory from persistent memory and are analyzed to determine which loci the set of blocks represent. An array of discrete attribute values across the plurality of probe spots, for each of the loci encoded in the set of blocks, is determined and used calculate the variance, or other needed parameters, for these loci across the plurality of probe spots. This process is repeated in which new set of blocks is loaded into non-persistent memory from persistent memory, analyzed to determine which loci are encoded in the new set of blocks, and then used to compute the variance, or other needed parameters, for these loci across the plurality of probe spots for each of the loci encoded in the new set of blocks, before discarding the set of blocks from non-persistent memory. In this way, only a limited amount of the discrete attribute value dataset 120 is stored in non-persistent memory 111 at any given time (e.g., the data for a particular block that contain the discrete attribute values for a particular locus). Further, the systems and methods of the present disclosure are able to compute variance in discrete attribute values for a given locus because it has got stored the discrete attribute values for that particular locus across one or more images and/or one or more regions of interest 121 of the discrete attribute value dataset 120 stored in a single bgzf block, in some embodiments. Once the variance, or other needed parameter is computed for the loci (or discrete attribute values of the loci), the accessed set of bgzf blocks (which is a subset of the total number of bgzf blocks in the dataset), which had been loaded into non-persistent memory 111 to perform the computation, is dropped from non-persistent memory and another set of bgzf blocks for which such computations is to be performed is loaded into the non-persistent memory 111 from the persistent memory 112. In some embodiments, such processes run in parallel (e.g., one process for each locus) when there are multiple processing cores 102. That is, each processing core concurrently analyzes a different respective set of blocks in the dataset and computes loci statistics for those loci represented in the respective set of blocks.

Following such normalization, in some embodiments, for each respective locus 122, an average (or some other measure of central tendency) discrete attribute value 124 (e.g., count of the locus 122) for each locus 122 is calculated for each cluster 158 of probe spots 126. Thus, in the case where there is a first and second cluster 158 of probe spots 126, the average (or some other measure of central tendency) discrete attribute value 124 of the locus A across all the probe spots 126 of the first cluster 158, and the average (or some other measure of central tendency) discrete attribute value 124 of locus A across all the probe spots 126 of the second cluster 158 is calculated and, from this, the differential value 162 for each the locus with respect to the first cluster is calculated. This is repeated for each of the loci 122 in a given cluster. It is further repeated for each cluster 158 in the plurality of clusters. In some embodiments, there are other factors that are considered, like adjusting the initial estimate of the variance in the discrete attribute value 124 when the data proves to be noisy. In the case where there are more than two clusters, the average (or some other measure of central tendency) discrete attribute value 124 of the locus A across all the probe spots 126 of the first cluster 158 and the average (or some other measure of central tendency) discrete attribute value 124 of locus A across all the probe spots 126 of the remaining cluster 158, is calculated and used to compute the differential value 162.

### Example 6 - Display a Heat Map

In some embodiments, the techniques of this Example 6 are run on any of the discrete attribute value datasets of the present disclosure.

With reference to FIG. 4, once the differential value 162 for each respective locus 122 in the plurality of loci for each respective cluster 158 in the plurality of clusters has been computed in accordance with Example 5, a heat map 402 of these differential values is displayed in a first panel 404 of an interface 400. The heat map 402 comprises a representation of the differential value 162 for each respective locus 122 in the plurality of loci for each cluster 158 in the plurality of clusters. As illustrated in FIG. 4, the differential value 162 for each locus 122 in the plurality of probe spots (e.g., loci from 122-1 to 122-M) for each cluster 158 (e.g., clusters 158-1, and 158-11) is illustrated in a color coded way to represent the log₂ fold change in accordance with color key 408. In accordance with color key 408, those loci 122 that are upregulated in the probe spots of a particular cluster 158 relative to all other clusters are assigned more positive values, whereas those loci 122 that are down-regulated in the probe spots of a particular cluster 158 relative to all other clusters are assigned more negative values. In some embodiments, the heat map can be exported to persistent storage (e.g., as a PNG graphic, JPG graphic, or other file formats).

### Example 7 - Two Dimensional Plot of the Probe Spots in the Dataset

In some embodiments, the techniques of this Example 7 are run on any of the discrete attribute value datasets of the present disclosure.

With reference to FIG. 4, in some embodiments, a two-dimensional visualization of the discrete attribute value dataset 120 is also provided in a second panel 420. In some embodiments, the two-dimensional visualization in the second panel 420 is computed by a back end pipeline that is remote from visualization system 100 and is stored as two-dimensional data points 166 in the discrete attribute value dataset 120 as illustrated in FIG. 1B. In some embodiments, the two-dimensional visualization 420 is computed by the visualization system.

Because the initial data is sparse, in some embodiments, the two-dimensional visualization is prepared by computing a corresponding plurality of principal component values 164 for each respective probe spot 126 in the plurality of probe spots based upon respective values of the discrete attribute value 124 for each locus 122 in the respective probe spot 126. In some embodiments, the plurality of principal component values is ten. In some embodiments, the plurality of principal component values is between 5 and 100. In some embodiments, the plurality of principal component values is between 5 and 50. In some embodiments, the plurality of principal component values is between 8 and 35. In some embodiments, a dimension reduction technique is then applied to the plurality of principal components values for each respective probe spot 126 in the plurality of probe spots, thereby determining a two-dimensional data point 166 for each probe spot 126 in the plurality of probe spots. Each respective probe spot 126 in the plurality of probe spots is then plotted in the second panel based upon the two-dimensional data point for the respective probe spot.

For instance, one embodiment of the present disclosure provides a back end pipeline that is performed on a computer system other than the visualization system 100. The back end pipeline comprises a two stage data reduction. In the first stage, the discrete attribute values 124 (e.g., mRNA expression data) for each locus 122 in a probe spot 126 is treated as a high-dimensional data point. For instance, the data point is, in some embodiments, a one-dimensional vector that includes a dimension for each of the 19,000 - 20,000 genes in the human genome, with each dimension populated with the measured mRNA expression level for the corresponding gene. More generally, a one-dimensional vector includes a dimension for each discrete attribute value 124 of the plurality of loci, with each dimension populated with the discrete attribute value 124 for the corresponding locus 122. This data is considered somewhat sparse and so principal component analysis is suitable for reducing the dimensionality of the data down to ten dimensions in this example. In some embodiments, application of principal component analysis can drastically reduce (reduce by at least 5-fold, at least 10-fold, at least 20-fold, or at least 40-fold) the dimensionality of the data (e.g., from approximately 20,000 to ten dimensions). That is, principal component analysis is used to assign each respective probe spot those principal components that describe the variation in the respective probe spot’s mRNA expression levels with respect to expression levels of corresponding mRNA of other probe spots in the dataset. Next, the data reduction technique t-Distributed Stochastic Neighboring Entities (t-SNE) is used to further reduce the dimensionality of the data from ten to two. t-SNE is a machine learning algorithm that is used for dimensionality reduction. See van der Maaten and Hinton, 2008, “Visualizing High-Dimensional Data Using t-SNE,” Journal of Machine Learning Research 9, 2579-2605, which is hereby incorporated by reference. The nonlinear dimensionality reduction technique t-SNE is particularly well-suited for embedding high-dimensional data (here, the ten principal components values 164) computed for each measured probe spot based upon the measured discrete attribute value (e.g., expression level) of each locus 122 (e.g., expressed mRNA) in a respective probe spot as determined by principal component analysis into a space of two, which can then be visualized as a two-dimensional visualization (e.g., the scatter plot of second panel 420). In some embodiments, t-SNE is used to model each high-dimensional object (the 10 principal components of each measured probe spot) as a two-dimensional point in such a way that similarly expressing probe spots are modeled as nearby two-dimensional data points 166 and dissimilarly expressing probe spots are modeled as distant two-dimensional data points 166 in the two-dimensional plot. The t-SNE algorithm comprises two main stages. First, t-SNE constructs a probability distribution over pairs of high-dimensional probe spot vectors in such a way that similar probe spot vectors (probe spots that have similar values for their ten principal components and thus presumably have similar discrete attribute values 124 across the plurality of loci 122) have a high probability of being picked, while dissimilarly dissimilar probe spot vectors (probe spots that have dissimilar values for their ten principal components and thus presumably have dissimilar discrete attribute values 124 across the plurality of loci 122) have a small probability of being picked. Second, t-SNE defines a similar probability distribution over the plurality of probe spots 126 in the low-dimensional map, and it minimizes the Kullback-Leibler divergence between the two distributions with respect to the locations of the points in the map. In some embodiments, the t-SNE algorithm uses the Euclidean distance between objects as the base of its similarity metric. In other embodiments, other distance metrics are used (e.g., Chebyshev distance, Mahalanobis distance, Manhattan distance, etc.).

In some embodiments, rather than using t-SNE, the dimension reduction technique used to reduce the principal component values 164 to a two-dimensional data point 166 is Sammon mapping, curvilinear components analysis, stochastic neighbor embedding, Isomap, maximum variance unfolding, locally linear embedding, or Laplacian Eigenmaps. These techniques are described in van der Maaten and Hinton, 2008, “Visualizing High-Dimensional Data Using t-SNE,” Journal of Machine Learning Research 9, 2579-2605, which is hereby incorporated by reference. In some embodiments, the user has the option to select the dimension reduction technique. In some embodiments, the user has the option to select the dimension reduction technique from a group comprising all or a subset of the group consisting of t-SNE, Sammon mapping, curvilinear components analysis, stochastic neighbor embedding, Isomap, maximum variance unfolding, locally linear embedding, and Laplacian Eigenmaps.

### Conclusion

The information types described above are presented on a user interface of a computing device in an interactive manner, such that the user interface can receive user input instructing the user interface to modify representation of the information. Various combinations of information can be displayed concurrently in response to user input. Using the information visualization methods described herein, previously unknown patterns and relationships can be discovered from discrete attribute value datasets. In this way, biological samples can be characterized.

All references cited herein are incorporated herein by reference in their entirety and for all purposes to the same extent as if each individual publication or patent or patent application was specifically and individually indicated to be incorporated by reference in its entirety for all purposes.

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subj ects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed:

1. A visualization system comprising one or more processing cores, a memory, and a display, the memory storing instructions for performing a method for evaluating one or more biological samples, the method comprising:
obtaining a discrete attribute value dataset derived by nucleic acid sequencing of the one or more biological samples, wherein the discrete attribute value dataset comprises a corresponding discrete attribute value for each reference sequence in a plurality of reference sequences for each respective entity in a plurality of entities in the one or more biological samples, wherein the plurality of entities comprises 100,000 entities;
indexing a two-dimensional spatial arrangement of the plurality of entities, in which each respective entity in the plurality of entities is independently assigned a unique two-dimensional position, in a k-dimensional binary search tree;
displaying the two-dimensional spatial arrangement of the plurality of entities on the display;
receiving a user selection of a subset of the two-dimensional spatial arrangement on the display;
determining each entity in the plurality of entities that is a member of the subset using the k-dimensional binary search tree, thereby identifying a subset of entities in the plurality of entities;
assigning each entity in the subset of entities to a user provided category; and
modifying the discrete attribute value dataset to store an association of each respective entity in the subset of entities to the user provided category.

2. The visualization system of claim 1, wherein the two-dimensional spatial arrangement of the plurality of entities on the display comprises 1,000 ,000 pixel values.

3. The visualization system of claim 1, wherein the method further comprises:
clustering the discrete attribute value dataset using the discrete attribute value for each reference sequence in the plurality of reference sequences, or a plurality of dimension reduction components derived therefrom, for each entity in the plurality of entities thereby assigning each respective entity in the plurality of entities to a corresponding cluster in a plurality of clusters; and
arranging the plurality of entities into the two-dimensional spatial arrangement based on the clustering.

4. The visualization system of claim 3, wherein each respective cluster in the plurality of clusters consists of a unique different subset of the plurality of entities.

5. The visualization system of claim 3, wherein the method further comprises:
assigning each respective cluster in the plurality of clusters a different graphic or color code, and
coloring each respective entity in the two-dimensional spatial arrangement of the plurality of entities in accordance with the different graphic or color code associated with the respective cluster corresponding to the respective entities.

6. The visualization system of claim 3, wherein the clustering the discrete attribute value dataset comprises hierarchical clustering, agglomerative clustering using a nearest-neighbor algorithm, agglomerative clustering using a farthest-neighbor algorithm, agglomerative clustering using an average linkage algorithm, agglomerative clustering using a centroid algorithm, or agglomerative clustering using a sum-of-squares algorithm.

7. The visualization system of claim 3, wherein the clustering the discrete attribute value dataset comprises application of a Louvain modularity algorithm, k-means clustering, a fuzzy k-means clustering algorithm, or Jarvis-Patrick clustering.

8. The visualization system of claim 3, wherein the clustering the discrete attribute value dataset comprises k-means clustering of the discrete attribute value dataset into a predetermined number of clusters.

9. The visualization system of claim 3, wherein the clustering the discrete attribute value dataset comprises k-means clustering of the discrete attribute value dataset into a number of clusters, wherein the number is acquired based on user input.

10. The visualization system of claim 1, wherein each reference sequence in the plurality of reference sequences is a different promoter, enhancer, silencer, insulator, mRNA, microRNA, piRNA, structural RNA, regulatory RNA, exon, or polymorphism.

11. The visualization system of claim 1, wherein the discrete attribute value dataset represents a transcriptome sequencing that quantifies gene expression from a single entity in counts of transcript reads mapped to genes.

12. The visualization system of claim 1, wherein each corresponding discrete attribute value is a count of a number of unique sequence reads in a plurality of sequence reads from the corresponding entities that have the reference sequence and a unique barcode associated with the corresponding entities.

13. The visualization system of claim 12, wherein the plurality of sequence reads comprises 100,000 sequence reads.

14. The visualization system of claim 12, wherein the plurality of sequence reads comprises 1,000,000 sequence reads.

15. The visualization system of claim 1, wherein the receiving the user selection of the subset of the two-dimensional spatial arrangement on the display comprises obtaining a closed form shape drawn by a user on the display that is within or overlaps the two-dimensional spatial arrangement.

16. The visualization system of claim 15, wherein the subset is each entity in the plurality of entities that is outside the closed form shape.

17. The visualization system of claim 15, wherein the subset is each entity in the plurality of entities that is inside the closed form shape.

18. The visualization system of claim 1, wherein an entity is a cell.

19. The visualization system of claim 1, wherein an entity is a probe spot.

20. The visualization system of claim 1, wherein an entity is a nucleus.

21. A computer-readable storage medium storing one or more computer programs, the one or more computer programs comprising instructions that, when executed by an electronic device with one or more processors and a memory, cause the electronic device to perform a method for evaluating one or more biological samples, comprising:
obtaining a discrete attribute value dataset derived by nucleic acid sequencing of the one or more biological samples, wherein the discrete attribute value dataset comprises a corresponding discrete attribute value for each reference sequence in a plurality of reference sequences for each respective entity in a plurality of entities in the one or more biological samples, wherein the plurality of entities comprises 100,000 entities;
indexing a two-dimensional spatial arrangement of the plurality of entities, in which each respective entity in the plurality of entities is independently assigned a unique two-dimensional position, in a k-dimensional binary search tree;
displaying the two-dimensional spatial arrangement of the plurality of entities on the display;
receiving a user selection of a subset of the two-dimensional spatial arrangement on the display;
determining each entity in the plurality of entities that is a member of the subset using the k-dimensional binary search tree, thereby identifying a subset of entities;
assigning each entity in the subset of entities to a user provided category; and
modifying the discrete attribute value dataset to store an association of each respective entity in the subset of entities to the user provided category.

22. A method of evaluating one or more biological samples, the method comprising:
using a computer system comprising one or more processing cores, a memory, and a display:
obtaining a discrete attribute value dataset derived by nucleic acid sequencing of the one or more biological samples, wherein the discrete attribute value dataset comprises a corresponding discrete attribute value for each reference sequence in a plurality of reference sequences for each respective entity in a plurality of entities in the biological sample, wherein the plurality of entities comprises 100,000 entities;
indexing a two-dimensional spatial arrangement of the plurality of entities, in which each respective entity in the plurality of entities is independently assigned a unique two-dimensional position, in a k-dimensional binary search tree;
displaying the two-dimensional spatial arrangement of the plurality of entities on the display;
receiving a user selection of a subset of the two-dimensional spatial arrangement on the display;
determining each entity in the plurality of entities that is a member of the subset using the k-dimensional binary search tree, thereby identifying a subset of entities;
assigning each entity in the subset of entities to a user provided category; and
modifying the discrete attribute value dataset to store an association of each respective entity in the subset of entities to the user provided category.
